(19) United States
(12) Patent Application Publication
(10) Pub. No.: US 2023/0365675 A1
(43) Pub. Date: Nov. 16, 2023

(54) SINGLE DOMAIN ANTIBODIES AGAINST CD33

(71) Applicant: VOR BIOPHARMA INC., Cambridge, MA (US)

(72) Inventors: Sadik Kassim, Cambridge, MA (US); Julian Scherer, Cambridge, MA (US)

(73) Assignee: VOR BIOPHARMA INC., Cambridge, MA (US)

(21) Appl. No.: 18/026,036

(22) PCT Filed: Sep. 14, 2021
(86) PCT No.: PCT/US2021/050341

§ 371 Date: Mar. 13, 2023

Related U.S. Application Data
(60) Provisional application No. 63/078,134, filed on Sep. 14, 2020.

(51) Int. Cl. C07K 16/28; C07K 14/705; C07K 14/725; A61P 35/02; A61K 45/06; A61K 39/395; A61K 39/00

(52) U.S. Cl. CPC C07K 16/2803; C07K 14/70517; C07K 14/70521; C07K 14/70578; C07K 14/7051; A61P 35/02; A61K 45/06; A61K 39/3955; A61K 39/4611; A61K 39/4631; A61K 39/464411; A61K 2039/505; C07K 2317/24; C07K 2317/569; C07K 2319/33; A61K 2239/48

(57) ABSTRACT

The present disclosure includes antibodies that specifically CD33, as well as methods of making and using such antibodies.

### RELATED APPLICATION

This application claims the benefit under 35 U.S.C. 119(e) of U.S. provisional application No. 63/078,134, filed Sep. 14, 2020, which is incorporated by reference herein in its entirety.

### BACKGROUND

CD33, also known as Siglec (Sialic-acid-binding immunoglobulin-like lectin) is frequently expressed on acute myeloid leukemia (AML) cells. AML remains a major therapeutic challenge and an unmet need in hematologic oncology. AML is a disease resulting in uncontrollable accumulation of immature myeloid blasts in the bone marrow and peripheral blood, and the disease has multiple subtypes that contribute to the challenge in developing an encompassing targeted therapy. Although there is an increased understanding in the molecular genetics of the disease, there have been relatively few novel therapies approved for AML. Accordingly, there remains a need for novel therapeutics for AML.

### SUMMARY

The present disclosure provides novel, antibodies against CD33. The present disclosure also provides use of such antibodies to treat neoplastic diseases and malignancies of the blood, hematopoietic malignancies, that are associated with CD33 expression (e.g., acute myeloid leukemia (AML), myelodysplastic syndrome (MDS), multiple myeloma (MM)).

In one aspect, the present disclosure provides an anti-CD33 antibody, or antigen-binding fragment thereof, comprising an amino acid sequence selected from a group consisting of SEQ ID NO: 1-88. In another aspect, the present disclosure provides an anti-CD33 antibody, or antigen-binding fragment thereof, comprising a complementarity determining region (CDR) sequence encompassed within any one of SEQ ID NO: 1-88. In another aspect, the present disclosure provides an anti-CD33 antibody, or antigen-binding fragment thereof, comprising CDR1, CDR2, and CDR3 encompassed within any one of SEQ ID NO: 1, 9, 17, 25, 33, 41, 49, 57, 65, 73, or 81. In another aspect, the present disclosure provides an anti-CD33 antibody, or antigen-binding fragment thereof, comprising at least one CDR (e.g., CDR1, CDR2, and/or CDR3) depicted in any one of SEQ ID NO: 1-88. In another aspect, the present disclosure provides an anti-CD33 antibody, or antigen-binding fragment thereof, comprising at least one CDR that is at least 85%, 90%, 91%, 92%, 93%, 94%, 95%, 96%, 97%, 98%, 99%, or 100% identical to a CDR (e.g., CDR1, CDR2, and/or CDR3) depicted in any one of SEQ ID NO: 1-88. In another aspect, the present disclosure provides an anti-CD33 antibody, or antigen-binding fragment thereof, comprising a heavy chain variable domain, e.g., a single domain antibody VHH, comprising an amino acid sequence selected from a group consisting of SEQ ID NO: 1-88. In another aspect, the present disclosure provides an anti-CD33 antibody, or antigen-binding fragment thereof, comprising a VHH comprising a CDR sequence encompassed within any one of SEQ ID NO: 1-88. In another aspect, the present disclosure provides an anti-CD33 antibody, or antigen-binding fragment thereof, comprising a VHH comprising CDR1, CDR2, and CDR3 encompassed within any one of SEQ ID NO: 1, 9, 17, 25, 33, 41, 49, 57, 65, 73, or 81. In another aspect, the present disclosure provides an anti-CD33 antibody, or antigen-binding fragment thereof, comprising a VHH comprising at least one CDR (e.g., CDR1, CDR2, and/or CDR3) depicted in any one of SEQ ID NO: 1-88. In another aspect, the present disclosure provides an anti-CD33 antibody, or antigen-binding fragment thereof, comprising a VHH comprising at least one CDR that is at least 85%, 90%, 91%, 92%, 93%, 94%, 95%, 96%, 97%, 98%, 99%, or 100% identical to a CDR (e.g., CDR1, CDR2, and/or CDR3) depicted in any one of SEQ ID NO: 1-88.

In some embodiments, an anti-CD33 antibody, or antigen-binding fragment thereof, is a monoclonal antibody, or antigen-binding fragment thereof. In some embodiments, an anti-CD33 antibody, or antigen-binding fragment thereof, is a humanized antibody, or antigen-binding fragment thereof. In some embodiments, an anti-CD33 antibody, or antigen-binding fragment thereof, competes with the antibody, or antigen-binding fragment thereof, described herein. In some embodiments, the anti-CD33 antibody, or antigen-binding fragment thereof, comprises a heavy chain variable region and one or more additional regions, such as one or more constant regions. In some embodiments, the anti-CD33 antibody, or antigen-binding fragment thereof, is a single domain antibody comprising a heavy chain variable domain, which may be referred to VHH. In some embodiments, the anti-CD33 antibody, or antigen-binding fragment thereof, is a single domain antibody consisting of a heavy chain variable domain.

In some embodiments, the anti-CD33 antibody, or antigen-binding fragment thereof, comprises a CH2 constant domain and a CH3 constant domain. In some embodiments, the anti-CD33 antibody, or antigen-binding fragment thereof, comprises an amino acid sequence of SEQ ID NO: 89. In some embodiments, the anti-CD33 antibody, or antigen-binding fragment thereof, is a heavy chain antibody. In some embodiments, the anti-CD33 antibody, or antigen-binding fragment thereof, is a camelid antibody.

Aspects of the present disclosure also provide chimeric antigen receptors comprising any of the antibodies, or antigen-binding fragment thereof, described herein. Aspects of the present disclosure also provide cells expressing any of the chimeric antigen receptors described herein. In some embodiments, the cell is an immune effector cell. In some embodiments, the cell is a lymphocyte. In some embodiments, the cell is a T-cell. In some embodiments, the cell is an NK cell.

In another aspect, the present disclosure provides a nucleic acid, comprising a nucleic acid sequence encoding any of the antibodies, or antigen-binding fragments thereof, described herein, or any of the chimeric antigen receptors described herein.

In another aspect, the present disclosure provides a vector comprising any of the nucleic acids described herein.

In another aspect, the present disclosure provides host cells comprising any of the nucleic acids described herein or any of the vectors described herein. In some embodiments, the host cell is an immune cell is selected from the group consisting of a T cell, a Natural Killer (NK) cell, a cytotoxic T lymphocyte (CTL), and a regulatory T cell.

In another aspect, the present disclosure provides methods of producing an antibody, or antigen-binding fragment thereof, comprising culturing any of the host cells described herein under conditions suitable for expression of the antibody or antigen-binding fragment thereof.

In another aspect, the present disclosure provides methods of treating a CD33-associated disease or disorder comprising administering to a subject in need thereof an effective amount of any of the antibodies, or antigen-binding fragments thereof, described herein, or any of the chimeric antigen receptors described herein. In another aspect, the present disclosure provides methods of treating a subject having or at risk of a neoplastic disease or malignancy of the blood that is associated with CD33 expression. In some embodiments, the methods comprise administering to the subject a therapeutically effective amount of an antibody, or antigen-binding fragment thereof, described herein. In some embodiments, the disease or malignancy is a hematopoietic malignancy. In some embodiments, a neoplastic disease or malignancy of the blood that is associated with CD33 expression is myelodysplastic syndrome (MDS), acute myeloid leukemia (AML), multiple myeloma (MM), or a combination thereof.

In some embodiments, the method of the present disclosure further comprises administering to the subject an effective amount of a chemotherapeutic agent or an oncolytic therapeutic agent. In some embodiments, method further comprises administering a population of hematopoietic cells, wherein the hematopoietic cells are genetically-engineered such that the gene encoding CD33 is engineered to reduce or eliminate the expression of CD33.

### BRIEF DESCRIPTION OF THE FIGURES

FIG. 1 shows an illustration of an Octet® assay for evaluating the epitope bound by exemplary anti-CD33 antibodies. Antibodies shown in contact with biotinylated CD33 are thought to bind to different epitopes on CD33, whereas the third antibody that is not in contact with biotinylated CD33 is thought to bind to the same epitope as one of the other antibodies and therefore is not able to interact with CD33.

FIG. 2 shows a graph of binding data from an Octet® assay evaluating competition between a first anti-CD33 antibody (a saturating antibody (mAb)) and a second anti-CD33 antibody (a competing antibody (mAb)).

FIG. 3 shows a heatmap representation of binding data from an Octet® assay evaluating competition between a first anti-CD33 antibody and a second anti-CD33 antibody.

FIG. 4 shows ribbon structures of CD33 showing three different structural representations depicting amino acids in CD33 targeted for mutation.

FIG. 5 shows (top) cell gating of 293FT cells transiently transfected with a mutant CD33-GFP fusion protein and (bottom) FACS data analyzing sdAb 348 or hu195 binding to exemplary CD33 mutant W22A.

FIG. 6 shows a heatmap representation of fluorescence-activated cell sorting (FACS) analysis data from mutant CD33-GFP binding experiments with ten sdAbs.

FIG. 7 shows protein structural diagrams of CD33 showing amino acids determined to be important for binding of each of the indicated anti-CD33 sdAbs tested and denoted above the diagrams.

FIG. 8 shows protein structural diagrams of CD33 showing amino acids determined to be important for binding of each of the indicated anti-CD33 sdAbs tested and denoted above the diagrams.

FIGS. 9A-9C show flow cytometry analysis plots of exemplary reporter cells as described herein. FIG. 9A shows Jurkat cells containing the mOrange reporter molecule under control of the constitutively active E1Falpha promoter and mTurquoise reporter molecule (mTurq) under control of an IL-2 reporter system described herein. Cells were either not activated (“−PMA/Ion,” top row) or activated using phorbol myristate acetate (PMA) and ionomycin (“+PMA/Ion,” bottom row). The left column of plots show cells expressing the mOrange reporter molecule; the middle column shows cells expressing the mTurquoise reporter molecule; and the right column shows cells expressing CD69, an indicator of T cell activation. FIG. 9B show Jurkat cells containing the mTurquoise reporter molecule (mTurq) under control of the constitutively active E1Falpha promoter and mOrange reporter molecule under control of an IL-2 reporter system described herein. Cells were either not activated (“−PMA/Ion,” top row) or activated using phorbol myristate acetate (PMA) and ionomycin (“+PMA/Ion,” bottom row). The left column of plots show cells expressing the mTurquoise reporter molecule; the middle column shows cells expressing the mOrange reporter molecule; and the right column shows cells expressing CD69, an indicator of T cell activation. FIG. 9C shows a plot of quantification flow cytometric analysis of FIGS. 9A and 9B. The y-axis shows the percentage of cells expressing the second reporter molecule (FP2), which was under control of an IL-2 reporter system described herein, based on the cells expressing the first reporter molecule (FP1), which was under control of the constitutively active promoter EF1a. Cells were either not activated (“−PMA/Ion”) or activated using phorbol myristate acetate (PMA) and ionomycin (“+PMA/Ion”). “EF1a_mOrange_IL-2_mTurq” refers to Jurkat cells containing the mOrange reporter molecule under control of the constitutively active E1Falpha promoter (FP1) and mTurquoise reporter molecule (mTurq) under control of an IL-2 reporter system described herein (FP2). “EF1a_mTurq_IL-2_mOrange” refers to Jurkat cells containing the mTurquoise reporter molecule under control of the constitutively active E1Falpha promoter (FP1) and mOrange reporter molecule under control of an IL-2 reporter system described herein (FP2).

FIG. 10 shows a graph of the fold increase in IL-2 inducible fluorescent protein (FP2; either mTurq in black or mOrange in light gray) upon exposure of Jurkat cells to MOLM13 CD33-expressing cells, where the Jurkat cells express the indicated CAR or co-stimulatory protein.

FIG. 11 shows a graph of the absolute change in IL-2 inducible fluorescence (ΔFP2) (either mTurq in black or mOrange in light gray) upon exposure of Jurkat cells to MOLM13 CD33-expressing cells, where the Jurkat cells express the indicated CAR or co-stimulatory protein.

FIGS. 12A and 12B show schematics of exemplary genetic constructs containing reporter molecules under control of the constitutive activate EF-1a promote FIG. 12A shows mOrange under control of the constitutive activate EF-1a promoter. FIG. 12B shows mTurquoise under control of the constitutive activate EF-1a promoter. These constructs provide constitutive expression of the relevant fluorescent protein in transfected cells.

FIGS. 13A and 13B show schematics of exemplary genetic constructs of the IL-2 reporter systems described herein. FIG. 13A shows the mOrange reporter molecule under control of a minimal NFAT-responsive promoter containing 6 NFAT binding sites and a minimal IL-2 promoter (“minP”) and the mTurquoise reporter molecule (mTurq) under control of the constitutively active E1Falpha promoter. FIG. 13B shows the mTurquoise reporter molecule under control of a minimal NFAT-responsive promoter containing 6 NFAT binding sites and a minimal IL-2 promoter (“minP”) and the mOrange reporter molecule under control of the constitutively active E1Falpha promoter. These constructs provide for expression of the reporter molecule under control of the IL-2 reporter system upon CAR activation, which may be assessed relative to expression of the reporter molecule under control of the constitutive promoter.

### DETAILED DESCRIPTION

The present disclosure is based, in part, on the discovery of novel antibodies that selectively bind to CD33. In some embodiments, the antibodies comprise a heavy chain variable domain. In some embodiments, the antibodies are single-domain antibodies. In some embodiments, the antibodies comprise a heavy chain variable domain and one or more constant domains. The disclosure also relates to nucleic acids encoding said antibodies, methods of producing said antibodies, and methods of use in the treatment of treat cancers (e.g., acute myeloid leukemia (AML), myelodysplastic syndrome (MDS)).

### Antibodies

The term “antibody” is used herein in the broadest sense and encompasses various antibody structures, including but not limited to monoclonal antibodies, polyclonal antibodies, multispecific antibodies (e.g., bispecific antibodies), and/or antibody fragments (preferably those fragments that exhibit the desired antigen-binding activity). An antibody described herein can be an immunoglobulin, heavy chain antibody, light chain antibody, LRR-based antibody, or other protein scaffold with antibody-like properties, as well as other immunological binding moiety known in the art, including, e.g., a Fab, Fab′, Fab′2, Fab2, Fab3, F(ab′)2, Fd, Fv, Feb, scFv, SMIP, antibody, diabody, triabody, tetrabody, minibody, Nanobody® (single domain antibody), maxibody, tandab, DVD, BiTe, TandAb, or the like, or any combination thereof. In some embodiments, the antibody is a heavy chain antibody. In some embodiments, the antibody is a camelid antibody. In some embodiments, the antibody comprises a heavy chain variable region and one or more constant regions (e.g., CH2 and CH3). In some embodiments, the antibody is a Nanobody®, also referred to as a single domain antibody or “VHH.” The subunit structures and three-dimensional configurations of different classes of antibodies are known in the art.

A “monoclonal antibody” or “mAb” refers to an antibody obtained from a population of substantially homogeneous antibodies, i.e., the individual antibodies comprising the population are identical and/or bind the same epitope, except for possible variant antibodies (e.g., containing naturally occurring mutations or arising during production of a monoclonal antibody preparation), such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

An “antigen-binding fragment” refers to a portion of an intact antibody that binds the antigen to which the intact antibody binds. An antigen-binding fragment of an antibody includes any naturally occurring, enzymatically obtainable, synthetic, or genetically engineered polypeptide or glycoprotein that specifically binds an antigen to form a complex. Exemplary antibody fragments include, but are not limited to, Fv, Fab, Fab′, Fab′-SH, F(ab′)2; diabodies; linear antibodies; single-chain antibody molecules (e.g. scFv or VHH or VH or VL domains only); and multispecific antibodies formed from antibody fragments. In some embodiments, the antigen-binding fragments of the antibodies described herein are scFvs. In some embodiments, the antigen-binding fragments of the antibodies described herein are VHH domains only. As with full antibody molecules, antigen-binding fragments may be mono-specific or multispecific (e.g., bispecific). A multispecific antigen-binding fragment of an antibody may comprise at least two different variable domains, wherein each variable domain is capable of specifically binding to a separate antigen or to a different epitope of the same antigen.

A “multispecific antibody” refers to an antibody comprising at least two different antigen binding domains that recognize and specifically bind to at least two different antigens. A “bispecific antibody” is a type of multispecific antibody and refers to an antibody comprising two different antigen binding domains that recognize and specifically bind to at least two different antigens.

A “different antigen” may refer to different and/or distinct proteins, polypeptides, or molecules; as well as different and/or distinct epitopes, which epitopes may be contained within one protein, polypeptide, or other molecule.

The term “epitope” refers to an antigenic determinant that interacts with a specific antigen binding site in the variable region of an antibody molecule known as a paratope. A single antigen may have more than one epitope. Thus, different antibodies may bind to different areas of an antigen and may have different biological effects. The term “epitope” also refers to a site of an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

As used herein, “selective binding”, “selectively binds” “specific binding”, or “specifically binds” refers, with respect to an antigen binding moiety and an antigen target, preferential association of an antigen binding moiety to an antigen target and not to an entity that is not the antigen target. A certain degree of non-specific binding may occur between an antigen binding moiety and a non-target. In some embodiments, an antigen binding moiety selectively binds an antigen target if binding between the antigen binding moiety and the antigen target is greater than 2-fold, greater than 5-fold, greater than 10-fold, or greater than 100-fold as compared with binding of the antigen binding moiety and a non-target. In some embodiments, an antigen binding moiety selectively binds an antigen target if the binding affinity is less than about 10⁻⁵ M, less than about 10⁻⁶ M, less than about 10⁻⁷ M, less than about 10⁻⁸ M, or less than about 10⁻⁹ M. In some embodiments, an antigen binding moiety selectively binds an epitope of an antigen target if binding between the antigen binding moiety and the epitope of the antigen target is greater than 2-fold, greater than 5-fold, greater than 10-fold, or greater than 100-fold as compared with binding of the antigen binding moiety and a non-target or another epitope of the antigen target. In some embodiments, an antigen binding moiety selectively binds an epitope of an antigen target if the binding affinity is less than about 10⁻⁵ M, less than about 10⁻⁶ M, less than about 10⁻⁷ M, less than about 10⁻⁸ M, or less than about 10⁻⁹ M.

In some embodiments, antibodies or fragments thereof that selectively bind to an identical epitope or overlapping epitope that will often cross-compete for binding to an antigen. Thus, in some embodiments, the disclosure provides an antibody or fragment thereof that cross-competes with an exemplary antibody or fragment thereof as disclosed herein. In some embodiments, to “cross-compete”, “compete”, “cross-competition”, or “competition” means antibodies or fragments thereof compete for the same epitope or binding site on a target. Such competition can be determined by an assay in which the reference antibody or fragment thereof prevents or inhibits specific binding of a test antibody or fragment thereof, and vice versa. Numerous types of competitive binding assays can be used to determine if a test molecule competes with a reference molecule for binding. Examples of assays that can be employed include solid phase direct or indirect radioimmunoassay (RIA), solid phase direct or indirect enzyme immunoassay (EIA), sandwich competition assay (see, e.g., Stahli et al. Methods in Enzymology (1983) 9:242-253), solid phase direct biotin-avidin EIA (see, e.g., Kirkland et al., J. Immunol. (1986) 137:3614-9), solid phase direct labeled assay, solid phase direct labeled sandwich assay, Luminex (Jia et al. “A novel method of Multiplexed Competitive Antibody Binning for the characterization of monoclonal antibodies” J. Immunological Methods (2004) 288, 91-98) and surface plasmon resonance (Song et al. “Epitope Mapping of Ibalizumab, a Humanized Anti-CD4 Monoclonal Antibody with Anti-HIV-1 Activity in Infected Patients” J. Virol. (2010) 84, 6935-42). In some embodiments, when a competing antibody or fragment thereof is present in excess, it will inhibit binding of a reference antibody or fragment thereof to a common antigen by at least 50%, 55%, 60%, 65%, 70%, or 75%. In some instances, binding is inhibited by at least 80%, 85%, 90%, 95%, 96%, 97%, 98%, 99%, or more.

An antibody can be an immunoglobulin molecule of four polypeptide chains, e.g., two heavy (H) chains and two light (L) chains. In some embodiments, a light chain is a lambda light chain. In some embodiments, a light chain is a kappa light chain. A heavy chain can include a heavy chain variable domain and a heavy chain constant domain. A heavy chain constant domain can include, any one or more of a CH1, hinge, CH2, CH3, and in some instances CH4 regions. A light chain can include a light chain variable domain and a light chain constant domain. A light chain constant domain can include a CL.

A heavy chain variable domain of a heavy chain and a light chain variable domain of a light chain can typically be further subdivided into regions of variability, termed complementarity determining regions (CDRs), interspersed with regions that are more conserved, termed framework regions (FR). In some embodiments, such heavy chain and/or light chain variable domains can each include three CDRs and four framework regions, arranged from amino-terminus to carboxyl-terminus in the following order: FR1, CDR1, FR2, CDR2, FR3, CDR3, FR4, one or more of which can be engineered as described herein. The CDRs in a heavy chain are designated “CDRH1”, “CDRH2”, and “CDRH3”, respectively, and the CDRs in a light chain are designated “CDRL1”, “CDRL2”, and “CDRL3”.

There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

Exemplary Single Domain Antibodies

Single domain antibodies are antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any known in the art, or any future single domain antibodies. Single domain antibodies may be derived from any species including, but not limited to mouse, human, camel, llama, goat, rabbit, and bovine. According to one aspect of the disclosure, a single domain antibody as used herein is a naturally occurring single domain antibody known as heavy chain antibody devoid of light chains. Such single domain antibodies are disclosed in, e.g., PCT Publication No. WO 94/04678. Such variable domains derived from a heavy chain antibody naturally devoid of light chain is referred to herein as a “VHH” or “Nanobody®”. Such a VHH can be derived from antibodies raised in Camelidae species, for example in camel, dromedary, llama, vicuna, alpaca and guanaco. Other species besides Camelidae may produce heavy chain antibodies naturally devoid of light chain; such VHHs are within the scope of the disclosure. In some embodiments, the antibody is a Nanobody® or “VHH” and comprises a heavy chain variable region. In some embodiments, the antibody comprises a heavy chain variable region and one or more heavy chain constant regions. In some embodiments, the antibody comprises a heavy chain variable region and does not comprise one or more heavy chain constant regions. In some embodiments, the antibody comprises a heavy chain variable region and does not comprise a light chain region (light chain variable region or light chain constant region).

The amino acid residues of VHH domains from Camelids are numbered according to the general numbering for VH domains given by Kabat et al., “Sequence of proteins of immunological interest”, US Public Health Services, NIH (Bethesda, MD), Publication No 91-3242 (1991); see also Riechmann et al., J. Immunol. Methods (1999) 231:25-38. According to this numbering, FR1 comprises the amino acid residues at positions 1-30, CDR1 comprises the amino acid residues at positions 31-35, FR2 comprises the amino acids at positions 36-49, CDR2 comprises the amino acid residues at positions 50-65, FR3 comprises the amino acid residues at positions 66-94, CDR3 comprises the amino acid residues at positions 95-102, and FR4 comprises the amino acid residues at positions 103-113.

It should be noted, however (as is well known in the art for VH domains and for VHH domains), that the total number of amino acid residues in each of the CDRs may vary and may not correspond to the total number of amino acid residues indicated by the Kabat numbering (that is, one or more positions according to the Kabat numbering may not be occupied in an actual sequence, or the actual sequence may contain more amino acid residues than the number allowed for by the Kabat numbering). This means that, generally, the numbering according to Kabat may or may not correspond to the actual numbering of the amino acid residues in an actual sequence.

Alternative methods for numbering the amino acid residues of VH domains, which methods can also be applied in an analogous manner to VHH domains, are known in the art. However, in the present disclosure, claims and figures, the numbering according to Kabat and applied to VHH domains as described above will be followed, unless indicated otherwise.

In some embodiments, the position numbering of an amino acid residue may be referred to based on a corresponding amino acid residue in a reference sequence.

The present disclosure provides antibodies that can include various heavy chains described herein. In some embodiments, an antibody comprises two heavy chains and light chains. In some embodiments, an antibody comprises two heavy chains, which may be two of the same heavy chain (having the same amino acid sequence) or different heavy chain (having a different amino acid sequence. In some embodiments, an antibody comprises two heavy chains, which may bind to the same epitope or a different epitope of a target antigen. In some embodiments, an antibody comprises two heavy chains, which may bind to epitopes of different target antigens. In some embodiments, the present disclosure encompasses an antibody including at least one heavy chain as disclosed herein, at least one heavy chain framework domain as disclosed herein, and/or at least one heavy chain CDR sequence as disclosed herein.

In some embodiments, an antibody disclosed herein is a homodimeric monoclonal antibody. In some embodiments, an antibody disclosed herein is a heterodimeric antibody. In some embodiments, an antibody is, e.g., a typical antibody or a diabody, triabody, tetrabody, minibody, Nanobody® (single domain antibody), maxibody, tandab, DVD, BiTe, scFv, TandAb scFv, Fab, Fab2, Fab3, F(ab′)2, or the like, or any combination thereof. In some embodiments, the antibody is a heavy chain antibody. In some embodiments, the antibody is a camelid antibody. In some embodiments, the antibody is a llama antibody. In some embodiments, the antibody comprises a heavy chain variable region and one or more constant regions. In some embodiments, the antibody is a Nanobody®, also referred to as a single domain antibody or “VHH.” In some embodiments, the antibody comprises one, two, or three immunoglobulin constant domains (e.g., chosen from CH1, CH2, CH3, and CH4). In some embodiments, the antibody comprises one, two, or three IgG1 constant domains. In some embodiments, the antibody comprises a CH2 and a CH3 domain. In some embodiments, the antibody comprises a CH fusion. An exemplary IgG1 CH2 and CH3 domain for use in an antibody of the disclosure is provided below:

(SEQ ID NO: 89)
APELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCS
VMHEALHNHYTQKSLSLSPGK

The present disclosure provides, among other things, an anti-CD33 antibody, or antigen-binding fragment thereof. In some embodiments, an anti-CD33 antibody, or antigen-binding fragment thereof, an amino acid sequence selected from a group consisting of SEQ ID NO: 1-88. In some embodiments, the anti-CD33 antibody, or antigen-binding fragment thereof, comprises a CDR sequence encompassed within any one of SEQ ID NOs: 1-88. In some embodiments, the anti-CD33 antibody, or antigen-binding fragment thereof, comprises a CDR1, CDR2, and CDR3 encompassed within any one of SEQ ID NOs: 1, 9, 17, 25, 33, 41, 49, 57, 65, 73, or 81. In some embodiments, the anti-CD33 antibody, or antigen-binding fragment thereof, comprises at least one CDR (e.g., CDR1, CDR2, and/or CDR3) depicted in any one of SEQ ID NO: 1-88. In some embodiments, the anti-CD33 antibody, or antigen-binding fragment thereof, comprises at least one CDR that is at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or 100% identical to a CDR (e.g., CDR1, CDR2, and/or CDR3) depicted in any one of SEQ ID NOs: 1-88.

The present disclosure provides, among other things, an anti-CD33 antibody, or antigen-binding fragment thereof comprising a VHH. In some embodiments, the anti-CD33 antibody, or antigen-binding fragment thereof, comprises a VHH comprising an amino acid sequence selected from a group consisting of SEQ ID NOs: 1-88. In some embodiments, the anti-CD33 antibody, or antigen-binding fragment thereof, comprises a VHH comprising a CDR sequence encompassed within any one of SEQ ID NOs: 1-88. In some embodiments, the anti-CD33 antibody, or antigen-binding fragment thereof, comprises a VHH comprising CDR1, CDR2, and CDR3 encompassed within any one of SEQ ID NOs: 1, 9, 17, 25, 33, 41, 49, 57, 65, 73, or 81. In some embodiments, the anti-CD33 antibody, or antigen-binding fragment thereof, comprises a VHH comprising at least one CDR (e.g., CDR1, CDR2, and/or CDR3) depicted in any one of SEQ ID NOs: 1-88. In some embodiments, the anti-CD33 antibody, or antigen-binding fragment thereof, comprises a VHH comprising at least one CDR that is at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a CDR (e.g., CDR1, CDR2, and/or CDR3) depicted in any one of SEQ ID NOs: 1-88.

In some embodiments, the anti-CD33 antibody, or antigen-binding fragment thereof, is a monoclonal antibody, or antigen-binding fragment thereof. In some embodiments, an anti-CD33 antibody, or antigen-binding fragment thereof, is a humanized antibody, or antigen-binding fragment thereof. In some embodiments, the anti-CD33 antibody, or antigen-binding fragment thereof, is a camelid antibody or has been derived from a camelid antibody. In some embodiments, the present disclosure provides an anti-CD33 antibody, or antigen-binding fragment thereof, that competes with an antibody, or antigen-binding fragment thereof, comprising an amino acid sequence selected from a group consisting of SEQ ID NOs: 1-88.

In some embodiments, the present disclosure provides an anti-CD33 antibody, or antigen-binding fragment thereof, comprising between 1 and 24 (e.g., 1, 2, 3, 4, 5, 10, or more) additions, deletions, or substitutions relative to an anti-CD33 antibody, or antigen-binding fragment thereof, wherein the anti-CD33 antibody comprises an amino acid sequence selected from a group consisting of SEQ ID NO: 1, 9, 17, 25, 33, 41, 49, 57, 65, 73, and 81 and, e.g., the antibody or fragment selectively binds CD33. In some embodiments, the present disclosure provides an anti-CD33 antibody, or antigen-binding fragment thereof, comprising an amino acid sequence having at least 80%, 85%, 90%, 91%, 92%, 93%, 94%, 95%, 96%, 97%, 98%, or 99% sequence identity to an amino acid sequence selected from a group consisting of SEQ ID NOs: 1, 9, 17, 25, 33, 41, 49, 57, 65, 73, and 81 and, e.g., the antibody or fragment selectively binds CD33.

The present disclosure provides, among other things, methods of making an anti-CD33 antibody, or antigen-binding fragment thereof. Methods of making antibodies are known in the art. For example, monoclonal antibodies can be produced using a variety of known techniques, such as the standard somatic cell hybridization technique described by Kohler and Milstein, Nature (1975) 256: 495. Other techniques for producing monoclonal antibodies also can be employed, e.g., viral or oncogenic transformation of B lymphocytes or phage display technique using libraries of human antibody genes.

In some embodiments, human antibodies are obtained by cloning the heavy and light chain genes directly from human B cells obtained from a human subject. The B cells are separated from peripheral blood (e.g., by flow cytometry, e.g., FACS), stained for B cell marker(s), and assessed for antigen binding. The RNA encoding the heavy and light chain variable regions (or the entire heavy and light chains) is extracted and reverse transcribed into DNA, from which the antibody genes are amplified (e.g., by PCR) and sequenced. The known antibody sequences can then be used to express recombinant human antibodies against a known target antigen. In some instances, human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extra-chromosomally or integrated randomly into the animal's chromosomes. In such transgenic mice, the endogenous immunoglobulin loci have generally been inactivated. Human variable regions from intact antibodies generated by such animals may be further modified, for example, by combining with a different human constant region.

In some instances, antibodies can also be made by hybridoma-based methods. In some embodiments, an animal system for generating hybridomas which produce human monoclonal antibodies is the murine system. Hybridoma production in the mouse is well known in the art, including immunization protocols and techniques for isolating and fusing immunized splenocytes. Human myeloma and mouse-human heteromyeloma cell lines for the production of human monoclonal antibodies have been described.

Human antibodies may also be generated by isolating Fv clone variable domain sequences selected from human-derived phage display libraries. Such variable domain sequences may then be combined with a desired human constant domain.

In some embodiments, the present disclosure provides methods of producing an antibody, or antigen-binding fragment thereof, comprising culturing a host cell comprising a nucleic acid encoding any of the anti-CD33 antibodies described herein. In some embodiments, the methods involve culturing a cell comprising a nucleic acid sequence selected from the group consisting of SEQ ID NOs: 1-88 under conditions suitable for expression of the antibody or antigen-binding fragment thereof. In some embodiments, the methods further comprise collecting, isolating, and/or purifying the antibodies or antigen-binding fragments thereof.

### Fusion Proteins and Conjugates

In some embodiments, the disclosure provides fusion proteins comprising (i) one or more single domain antibodies, or antigen-binding fragments thereof, described herein (e.g., comprising one or more CDRs described herein), and (ii) one or more additional polypeptides. In some embodiments, the disclosure provides fusion proteins comprising (i) one or more single domain antibodies, or antigen-binding fragments thereof, described herein (e.g., comprising one or more CDRs described herein), and (ii) one or more additional domains. For example, a fusion protein can include one or more single domain antibodies described herein and one or more (e.g., 1, 2, 3, 4 or more) constant regions or an Fc region. In some embodiments, one or more single domain antibodies, or antigen-binding fragments thereof, described herein (e.g., one or more CDRs described herein) can be conjugated non-covalently or covalently, e.g., fused, to an antigen (e.g., an antigen target for a cellular therapeutic, e.g., a CAR-T cell or an antibody drug conjugate) as described in, e.g., PCT Publication Nos. WO2017/075537, WO2017/075533, WO2018156802, and WO2018156791.

In some embodiments, the disclosure provides a fusion protein comprising one or more VHH as described herein and one or more additional polypeptides or polypeptide domains. In some embodiments, an additional polypeptide comprises an additional antibody or fragment thereof. Additional antibodies include, e.g., intact IgG, IgE and IgM, bi- or multi-specific antibodies (e.g., Zybodies®, etc), single chain Fvs, polypeptide-Fc fusions, Fabs, cameloid antibodies, masked antibodies (e.g., Probodies®), Small Modular ImmunoPharmaceuticals (“SMIPs™”), single chain or Tandem diabodies (TandAb®), VHHs (including but not limited to those described in the present disclosure), Anticalins®, Nanobodies®, minibodies, BiTE®s, ankyrin repeat proteins or DARPINs®, Avimers®, a DART, a TCR-like antibody, Adnectins®, Affilins®, Trans-Bodies®, Affibodies®, a TrimerX®, MicroProteins, Fynomers®, and Centyrins®.

In some embodiments, the one or more additional polypeptides or polypeptide domains comprises a second antigen-binding domain, such as a second antigen-binding domain that binds to the same target antigen (i.e., CD33), for example any of the anti-CD33 antibody, or antigen-binding fragments thereof, described herein. In some embodiments, the one or more additional polypeptides or polypeptide domains comprises a second antigen-binding domain, such as a second antigen-binding domain that binds to a different target antigen (e.g., not an epitope of CD33).

In some embodiments, an antibody of the disclosure can be covalently attached by linker (e.g., by a disulfide or non-cleavable thioether linker) to a drug (e.g., a cytotoxic agent, such as a toxin) as an antibody drug conjugate (ADC). The drug to which the antibody is covalently attached may have cytotoxic or cytostatic effect when it is not conjugated to the antibody. The ADC can be used to selectively delivery an effective dose of a cytotoxic agent to cells (e.g., to tumor tissue). The ADC may improve the bioavailability of the drug and/or the antibody compared to when the drug and/or antibody is administered in its unconjugated form.

A variety of linker types and strategies are known in the art and any or all of these are contemplated for use with the antibodies or ADCs of the disclosure. In some embodiments, the linker is biodegradable, e.g., cleavable by an endogenous protease (e.g., present in the target tissue and/or cells). In some embodiments the linker comprises a protease cleavable site. In some embodiments, the linker comprises a pH sensitive site, e.g., a site sensitive to acidic pH, e.g., that hydrolyzes under acidic conditions. In some embodiments, the linker is stable under physiological conditions, e.g., sufficiently stable so that the antibody targets the drug to the target tissue prior to release of the drug. In some embodiments, the linker comprises a disulfide bond, e.g., a glutathione-sensitive disulfide bond. In some embodiments, the drug conjugated to the antibody is active only after cleavage of the linker. In some embodiments, the drug conjugated to the antibody is active only after proteolytic digestion of the antibody (e.g., in the lysosome of a target cell). In some embodiments, the linker is a non-cleavable heterobifunctional thiooether linker, e.g., a maleimide linker, e.g., comprising N-hydroxysuccinimide ester (succinimidyl-4-(N-maleimidomethyl) cyclohexane-1-carboxylate or SMCC.

A variety of drugs compatible with ADCs of the disclosure are known in the art and any or all of these are contemplated for use with the antibodies of the disclosure.

Also within the scope of the present disclosure are chimeric antigen receptors (CARs) comprising any of the anti-CD33 antibodies, or antigen-binding fragments thereof, described herein. A CAR is an artificially constructed hybrid protein or polypeptide containing the antigen-binding domain of one or more antibodies (e.g., single chain variable fragment (scFv)) linked to T-cell signaling domains. Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains. The phrases “antigen(ic) specificity” and “elicit antigen-specific response,” as used herein, means that the CAR can specifically bind to and immunologically recognize antigen, such that binding of the CAR to the antigen elicits an immune response.

Of the conventional CARs containing an antigen-binding domain of an antibody, there are three generations of CARs. “First generation” CARs are typically composed of an extracellular antigen-binding domain (e.g., a scFv), which is fused to a transmembrane domain, which is fused to cytoplasmic/intracellular signaling domain. First generation CARs can provide de novo antigen recognition and cause activation of both CD4+ and CD8+ T cells through their CD3ζ chain signaling domain in a single fusion molecule, independent of HLA-mediated antigen presentation. “Second generation” CARs add an intracellular signaling domain from various co-stimulatory signaling molecules (e.g., CD28, 4-1BB, ICOS, 0X40, CD27, CD40/My88 and NKGD2) to the cytoplasmic tail of the CAR to provide additional signals to the T cell. Second generation CARs comprise those that provide both co-stimulation (e.g., CD28 or 4-1BB) and activation (CD3ζ). “Third generation” CARs comprise those that provide multiple co-stimulatory domains (e.g., CD28 and 4-1BB) and a signaling domain providing activation (e.g., CD3ζ).

The CARs described herein comprise an extracellular portion of the CAR containing anti-CD33 binding fragment, a transmembrane domain, and a signaling domain. In some embodiments, the CAR further comprises one or more of a linker region, hinge region, and co-stimulatory signaling domains. In some embodiments, the CAR further comprises a signal peptide/signal sequence.

A CAR can consist of or consist essentially of the specified amino acid sequence or sequences described herein, such that other components, e.g., other amino acids, do not materially change the biological activity of the functional variant.

CARs of the present disclosure (including functional portions and functional variants) can be of any length, i.e., can comprise any number of amino acids, provided that the CARs or functional portions or functional variants thereof) retain their biological activity, e.g., the ability to specifically bind to the target antigen (e.g., CD33), detect diseased cells in a mammal, or treat or prevent disease in a mammal, etc. For example, the CAR can be about 50 to about 5000 amino acids long, such as 50, 70, 75, 100, 125, 150, 175, 200, 300, 400, 500, 600, 700, 800, 900, 1000 or more amino acids in length.

In some embodiments, CAR constructs (including functional portions and functional variants of the invention) can comprise synthetic amino acids in place of one or more naturally-occurring amino acids. Such synthetic amino acids are known in the art, and include, for example, aminocyclohexane carboxylic acid, norleucine, a-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, b-phenylserine b-hydroxyphenylalanine, phenylglycine, a-naphthylalanine, cyclohexylalanine, cyclohexylglycine, indoline-2-carboxylic acid, 1,2, 3, 4-tetrahydroisoquinoline-3-carboxylic acid, aminomalonic acid, aminomalonic acid monoamide, N′-benzyl-N′ 6-hydroxylysine, ornithine, a-aminocyclopentane carboxylic acid, a-aminocyclohexane carboxylic acid, a-aminocycloheptane carboxylic acid, a-(2-amino-2-norbornane)-carboxylic acid, a,g-diaminobutyric acid, a,b-diaminopropionic acid, homophenylalanine, and a-tert-butylglycine.

In some embodiments, CAR constructs (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

In some embodiments, CAR constructs (including functional portions and functional variants thereof) can be obtained by methods known in the art. In some embodiments, CAR constructs may be made by any suitable method of making polypeptides or proteins, including de novo synthesis. CAR constructs can be recombinantly produced using the nucleic acids described herein using standard recombinant methods. See, for instance, Green et al., Molecular Cloning: A Laboratory Manual, 4th ed., Cold Spring Harbor Press, Cold Spring Harbor, N Y 2012. Further, portions of some of the CAR constructs described herein (including functional portions and functional variants thereof) can be isolated and/or purified from a source, such as a plant, a bacterium, an insect, a mammal, e.g., a rat, a human, etc. Methods of isolation and purification are well known in the art. Alternatively, the CAR constructs described herein (including functional portions and functional variants thereof) can be commercially synthesized by companies, such as Synpep (Dublin, CA), Peptide Technologies Corp. (Gaithersburg, MD), and Multiple Peptide Systems (San Diego, CA). In this respect, the CAR constructs can be synthetic, recombinant, isolated, and/or purified.

Further provided herein are nucleic acids comprising a nucleotide sequence encoding any of the CAR constructs described herein (including functional portions and functional variants thereof). The nucleic acids described herein may comprise a nucleotide sequence encoding any of the leader sequences (e.g., signal peptides), antigen binding domains, transmembrane domains, linker regions, costimulatory signaling domains, and/or intracellular T cell signaling domains described herein.

In some aspects, any of the antigen-binding domains described herein may be operably linked to another domain of the CAR, such as the transmembrane domain or the intracellular domain, for expression in the cell. In some embodiments, a nucleic acid encoding the antigen-binding domain is operably linked to a nucleic acid encoding a transmembrane domain and a nucleic acid encoding an intracellular domain.

In some embodiments, a nucleic acid encoding the anti-CD33 antigen binding domain is operably linked to a nucleic acid encoding a linker region, a nucleic acid encoding a transmembrane domain, and/or a nucleic acid encoding an intracellular domain (e.g., a costimulatory signaling domain, a signaling domain). In some embodiments, the CAR comprises any of the anti-CD33 antibodies or antigen-binding fragments thereof, described herein (e.g., comprising one or more CDRs described herein). In some embodiments, the CAR comprises any of the anti-CD33 antibodies or antigen-binding fragments thereof, provided in any one of SEQ ID NOs: 1, 9, 17, 25, 33, 41, 49, 57, 65, 73, 81.

In some embodiments, the CAR comprises a linker region. In some embodiments, the light chain variable region and the heavy chain variable region of the antigen-binding domain can be joined to each other by a linker. In some embodiments, the antigen-binding domain can be joined to another domain, such as a transmembrane domain, hinge, and/or intracellular domain with a linker region. The linker may comprise any suitable amino acid sequence. In some embodiments, the linker is a Gly/Ser linker from about 1 to about 100, from about 3 to about 20, from about 5 to about 30, from about 5 to about 18, or from about 3 to about 8 amino acids in length and consists of glycine and/or serine residues in sequence. Accordingly, the Gly/Ser linker may consist of glycine and/or serine residues. Preferably, the Gly/Ser linker comprises the amino acid sequence of GGGGS (SEQ ID NO: 100), and multiple SEQ ID NO: 100 may be present within the linker. Any linker sequence may be used as a spacer between the antigen-binding domain and any other domain of the CAR, such as the transmembrane domain. In some, embodiments, the region linker is ([G]x[S]y)z (SEQ ID NO: 111), for example wherein x can be 1-10, y can be 1-3, and z can be 1-5. In some embodiments, the linker region comprises the amino acid sequence GGGGSGGGGS (SEQ ID NO: 101). In some embodiments, the linker region comprises the amino acid sequence GGGGSGGGGSGGGGS (SEQ ID NO: 102).

In some embodiments, the antigen-binding domain comprises one or more leader sequences (signal peptides, signal sequence), such as those described herein. In some embodiments, the leader sequence may be positioned at the amino terminus of the CAR within the CAR construct. The leader sequence may comprise any suitable leader sequence, e.g., any CARs described herein may comprise any leader sequence, such as those described herein. In some embodiments, while the leader sequence may facilitate expression of the released CARs on the surface of the cell, the presence of the leader sequence in an expressed CAR is not necessary in order for the CAR to function. In some embodiments, upon expression of the CAR on the cell surface, the leader sequence may be cleaved off. Accordingly, in some embodiments, the released CARs (e.g., surface expressed) lack a leader sequence. In some embodiments, the CARs within the CAR construct lack a leader sequence.

Hinge

In some embodiments, the CAR comprises a hinge/spacer region that links the extracellular antigen-binding domain to another domain, such as a transmembrane domain. The hinge/spacer region can be flexible enough to allow the antigen-binding domain to orient in different directions to facilitate target antigen recognition. In some embodiments, the hinge domain is a portion of the hinge domain of CD8a or CD28, e.g., a fragment containing at least 15 (e.g., 20, 25, 30, 35, or 40) consecutive amino acids of the hinge domain of CD8a or CD28.

In some embodiments, the CAR comprises a hinge domain, such as a hinge domain from CD8, CD28, or IgG4. In some embodiments, the hinge domain is a CD8 (e.g., CD8a) hinge domain. In some embodiments, the CD8 hinge domain is human (e.g., obtained from/derived from a human protein sequence). In some embodiments, the CD8 hinge domain comprises, consists of, or consists essentially of SEQ ID NO: 103.

CD8 hinge region
[SEQ ID NO: 103]
TTTPAPRPPTPAPTIASQPLSLRPEACRPAAGGAVHTRGLDFACD

In some embodiments, the hinge domain is a CD28 hinge domain. In some embodiments, the CD28 hinge domain is human (e.g., obtained from/derived from a human protein sequence). In some embodiments, the CD28 hinge domain comprises, consists of, or consists essentially of SEQ ID NO: 104.

CD28 hinge region
[SEQ ID NO: 104]
AAAIEVMYPPPYLDNEKSNGTIIHVKGKHLCPSPLFPGPSKP

Hinge domains of antibodies, such as an IgG, IgA, IgM, IgE, or IgD antibody, are also compatible for use in the chimeric receptors described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody. In some embodiments, the hinge domain is an IgG4 hinge domain.

Also within the scope of the present disclosure are CARs comprising a hinge domain that is a non-naturally occurring peptide. In some embodiments, the hinge domain between the C-terminus of the extracellular ligand-binding domain of an Fc receptor and the N-terminus of the transmembrane domain is a peptide linker, such as a (GlyxSer)n (SEQ ID NO: 105) linker, wherein x and n, independently can be an integer between 3 and 12, including 3, 4, 5, 6, 7, 8, 9, 10, 11, 12, or more.

Additional peptide linkers that may be used in a hinge domain of the chimeric receptors described herein are known in the art. See, e.g., Wriggers et al. Current Trends in Peptide Science (2005) 80(6): 736-74 and PCT Publication No. WO 2012/088461.

In some embodiments, the hinge/spacer region of a presently disclosed CAR comprises a native or modified hinge region of a CD28 polypeptide as described herein. In certain embodiments, the hinge/spacer region of a presently disclosed CAR construct comprises a native or modified hinge region of a CD8α polypeptide as described herein. In certain embodiments, the hinge/spacer region of a presently disclosed CAR construct comprises a native or modified hinge region of a IgG4 polypeptide as described herein.

Transmembrane Domain

With respect to the transmembrane domain, a CAR can be designed to comprise a transmembrane domain that connects the antigen-binding domain of the CAR to an intracellular region of the CAR. In some embodiments, the transmembrane domain is naturally associated with one or more of the domains in the CAR. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of the alpha, beta or zeta chain of the T-cell receptor, CD28, CD3 epsilon, CD45, CD4, CD5, CD8, CD8a, CD9, CD 16, CD22, CD33, CD37, CD64, CD80, CD86, CD134, CD137, CD154, Toll-like receptor 1 (TLR1), TLR2, TLR3, TLR4, TLR5, TLR6, TLR7, TLR8, and TLR9.

In some embodiments, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

In some embodiments, the transmembrane domain is a CD8 (e.g., CD8a) transmembrane domain. In some embodiments, the CD8 transmembrane domain is human (e.g., obtained from/derived from a human protein sequence). In some embodiments, a CD8 transmembrane domain comprises, consists of, or consists essentially of SEQ ID NO: 106.

CD8 transmembrane region
[SEQ ID NO: 106]
IYIWAPLAGTCGVLLLSLVITLYC

In some embodiments, the transmembrane domain is a CD28 transmembrane domain. In some embodiments, the CD28 transmembrane domain is human (e.g., obtained from/derived from a human protein sequence). In some embodiments, the CD28 transmembrane domain comprises, consists of, or consists essentially of SEQ ID NO: 107.

CD28 transmembrane domain
[SEQ ID NO: 107]
FWVLVVVGGVLACYSLLVTVAFIIFWVRSKRSRLLHSDYMNMTPRRPGP
TRKHYQPYAPPRDFAAYRS

Intracellular Signaling Domains

In some embodiments, the CAR construct comprises an intracellular signaling domain, which may be comprised of one or more signaling domains and costimulatory domains. The intracellular signaling domain of the CAR, is involved in activation of the cell in which the CAR is expressed. In some embodiments, the intracellular signaling domain of the CAR construct described herein is involved in activation of a T lymphocyte or NK cells. In some embodiments, the signaling domain of the CAR construct described herein includes a domain involved in signal activation and/or transduction.

Examples of an intracellular signaling domains for use in the CAR constructs described herein include, but are not limited to, the cytoplasmic portion of a surface receptor, co-stimulatory molecule, and any molecule that acts in concert to initiate signal transduction in a cell (e.g., an immune cell (e.g., a T lymphocyte), NK cell), as well as any derivative or variant of these elements and any synthetic sequence that has the same functional capability.

Examples of the signaling domains that may be used in the intracellular signaling domain of the CARs described herein include, without limitation, a fragment or domain from one or more molecules or receptors including, but are not limited to, TCR, CD3 zeta (CD3ζ), CD3 gamma, CD3 delta, CD3 epsilon, CD86, common FcR gamma, FcR beta (Fc Epsilon Rib), CD79a, CD79b, Fcgamma RIIa, DAP10, DAP 12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD 1 id, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Any cytoplasmic signaling domain can be used in the CARs described herein. In general, a cytoplasmic signaling domain relays a signal, such as interaction of an extracellular ligand-binding domain with its ligand, to stimulate a cellular response, such as inducing an effector function of the cell (e.g., cytotoxicity).

As will be evident to one of ordinary skill in the art, a factor involved in T cell activation is the phosphorylation of immunoreceptor tyrosine-based activation motif (ITAM) of a cytoplasmic signaling domain. Any ITAM-containing domain known in the art may be used to construct the chimeric receptors described herein, and included as part of the cytoplasmic signaling domain. In general, an ITAM motif may comprise two repeats of the amino acid sequence YxxL/I separated by 6-8 amino acids, wherein each x is independently any amino acid, producing the conserved motif YxxL/Ix(6-8)YxxL/I. In some embodiments, the cytoplasmic signaling domain is from CD3ζ.

CD3ζ associates with TCRs to produce a signal and contains immunoreceptor tyrosine-based activation motifs (ITAMs). In some embodiments, a CD3ζ intracellular T cell signaling sequence is human (e.g., obtained from or derived from a human protein). In some embodiments, a CD3ζ intracellular T cell signaling sequence comprises, consists of, or consists essentially of the amino acid sequence of SEQ ID NO: 108 or 109, or a sequence that is at least 70%, at least 75%, at least 80%, at least 85%, at least 90%, at least 95%, or at least 99% identical the amino acid sequence of SEQ ID NO: 108 or 109. In some embodiments, an intracellular T cell signaling domain comprises a CD3ζ that contains on or more mutated and/or deleted ITAMs.

CD3 ζ signaling domain (variant A)
[SEQ ID NO: 108]
RVKF SRSADAPAYKQGQNQLYNELNLGRREEYDVLDKRRGRDPEMGGK
PRRKNPQEGLYNELQKDKMAEAYSEIGMKGERRRGKGHDGLYQGLSTAT
KDTYDALHMQALPPR
CD3 ζ signaling domain (variant B)
[SEQ ID NO: 109]
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

In certain non-limiting embodiments, an intracellular signaling domain of the CAR further comprises at least one (e.g., 1, 2, 3 or more) co-stimulatory signaling domain. In some embodiments, the co-stimulatory signaling domain comprises at least one co-stimulatory molecule, which can provide optimal lymphocyte activation. In general, many immune cells require co-stimulation, in addition to stimulation of an antigen-specific signal, to promote cell proliferation, differentiation and survival, and to activate effector functions of the cell. Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory protein may be compatible for use in the chimeric receptors described herein. The type(s) of co-stimulatory signaling domains may be selected based on factors such as the type of the cells in which the CARs would be expressed (e.g., primary T cells, T cell lines, NK cell lines) and the desired immune effector function (e.g., cytotoxicity).

Examples of such co-stimulatory signaling domains include a fragment or domain from one or more molecules or receptors including, without limitation, are not limited to 4-1BB, CD28, ICOS, TLR1, TLR2, TLR3, TLR4, TLR5, TLR6, TLR7, TLR8, TLR9, TLR10, TLR11, CD116 receptor beta chain, CSF1-R, LRP1/CD91, SR-A1, SR-A2, MARCO, SR-CL1, SR-CL2, SR-C, SR-E, CR1, CR3, CR4, dectin 1, DEC-205, DC-SIGN, CD14, CD36, LOX-1, CD11b, together with any of the signaling domains listed in the above paragraph in any combination. In some embodiments, the intracellular signaling domain of the CAR includes any portion of one or more co-stimulatory signaling molecules, such as at least one signaling domain from CD3, Fc epsilon RI gamma chain, any derivative or variant thereof, including any synthetic sequence thereof that has the same functional capability, and any combination thereof.

In some embodiments, one or more co-stimulatory signaling domains (e.g., 1, 2, 3, or more) are included in a CAR construct with a CD3ζ intracellular T cell signaling sequence. In some embodiments, the one or more co-stimulatory signaling domains are selected from CD137 (4-1BB) and CD28, or a combination thereof. In some embodiments, the CAR comprises a 4-1BB (CD137) costimulatory signaling domain. In some embodiments, the CAR comprises a CD28 costimulatory signaling domain. In some embodiments, the CAR comprises both a 4-1BB costimulatory signaling domain and a CD28 costimulatory signaling domain.

4-1BB, also known as CD137, transmits a potent costimulatory signal to T cells, promoting differentiation and enhancing long-term survival of T lymphocytes. In some embodiments, a 4-1BB intracellular signaling sequence is human (e.g., obtained from/derived from a human protein sequence). In some embodiments, the 4-1BB intracellular T cell signaling sequence comprises, consists of, or consists essentially of the amino acid sequence of SEQ ID NO: 110. In some embodiments, the 4-1BB costimulatory signaling domain comprises, consists of, or consists essentially of the amino acid sequence of SEQ ID NO: 110, or a sequence that is at least 70%, at least 75%, at least 80%, at least 85%, at least 90%, at least 95%, or at least 99% identical the amino acid sequence of SEQ ID NO: 110.

4-1BB costimulatory signaling domain
[SEQ ID NO: 110]
KRGRKKLLYIFKQPFMRPVQTTQEEDGCSCRFPEEEEGGCEL

Some suitable costimulatory domains are provided herein, and other suitable costimulatory domains and costimulatory domain sequences will be apparent to the skilled artisan based on the present disclosure in view of the knowledge in the art. Suitable costimulatory domains include, for example, those described in Weinkove et al., Selecting costimulatory domains for chimeric antigen receptors: functional and clinical considerations, Clin Transl Immunology. 2019; 8(5): e1049, the entire contents of which are incorporated herein by reference.

Between the antigen-binding domain and the transmembrane domain of the CAR, or between the intracellular signaling domain and the transmembrane domain of the CAR, a spacer domain may be incorporated. As used herein, the term “spacer domain” generally means any oligo- or polypeptide that functions to link the transmembrane domain to, either the antigen binding domain or, the intracellular domain in the polypeptide chain. In some embodiments, the spacer domain may comprise up to 300 amino acids, preferably 10 to 100 amino acids and most preferably 25 to 50 amino acids. In some embodiments, a short oligo- or polypeptide linker, preferably between 2 and 10 amino acids in length may form the linkage between the transmembrane domain and the intracellular domain of the CAR. An example of a linker includes a glycine-serine doublet.

### Signal Peptides

In some embodiments, any of the CARs described herein may further comprise a signal peptide (signal sequence). In general, signal peptides are short amino acid sequences that target a polypeptide to a site in a cell. In some embodiments, the signal peptide directs the CAR to the secretory pathway of the cell and will allow for integration and anchoring of the CAR into the lipid bilayer at the cell surface. Signal sequences including signal sequences of naturally occurring proteins or synthetic, non-naturally occurring signal sequences, that are compatible for use in the chimeric receptors described herein will be evident to one of skill in the art.

The CARs described herein may be prepared in constructs with, e.g., self-cleaving peptides, such that the CAR constructs containing anti-CD33 CAR components are bicistronic, tricistronic, etc.

Various CAR constructs and numerous elements of CAR constructs (for example, various CD33 binding domains, signal peptides, linkers, hinge sequences, transmembrane domains, costimulatory domains, and signaling domains) are disclosed herein, and those of skill in the art will be able to ascertain the sequences of these elements and of additional suitable elements known in the art based on the present disclosure in view of the knowledge in the art. Exemplary CAR element sequences, e.g., for CD33 binding domains, signal peptides, linkers, hinge sequences, transmembrane domains, costimulatory domains, and signaling domains, are disclosed in PCT/US2019/022309, published as WO/2019/178382, e.g., throughout the specification and in Tables 1-6, the entire contents of which are incorporated herein by reference.

CAR5

An exemplary CAR construct comprising one or more single domain antibodies, or antigen-binding fragments thereof, described herein comprises a CD33 binding domain as comprised in SEQ ID NO: 25, a CD8a transmembrane domain, a CD8a hinge domain, a CD137 (4-1BB) co-stimulatory domain, and a CD3ζ intracellular signaling domain.

In some embodiments, a CAR comprises an amino acid sequence shown in SEQ ID NO: 90, or an amino acid sequence that is at least 70%, at least 75%, at least 80%, at least 85%, at least 90%, at least 95%, or at least 99% identical to the amino acid sequence shown in SEQ ID NO: 90.

[SEQ ID NO: 90]
MELGLSWVVLAALLQGVQAQVKLEESGGGSVQAGESLRLSCTASGITFR
DYDIDWYRQAPGKEREWLATITPSGTTHYPDSVKGRATISRDSAKNTVY
LQMNSLKPEDTARYECNTLAYWGSGTQVTVSSAAATTTPAPRPPTPAPT
IASQPLSLRPEACRPAAGGAVHTRGLDFACDIYIWAPLAGTCGVLLLSL
VITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKD
TYDALHMQALPPR

CAR 6

An exemplary CAR construct comprising one or more single domain antibodies, or antigen-binding fragments thereof, described herein comprises a CD33 binding domain as comprised in SEQ ID NO: 73, a CD8a transmembrane domain, a CD8a hinge domain, a CD137 (4-1BB) co-stimulatory domain, and a CD3ζ intracellular signaling domain.

In some embodiments, a CAR comprises an amino acid sequence shown in SEQ ID NO: 91, or an amino acid sequence that is at least 70%, at least 75%, at least 80%, at least 85%, at least 90%, at least 95%, or at least 99% identical to the amino acid sequence shown in SEQ ID NO: 91.

[SEQ ID NO: 91]
MELGLSWVVLAALLQGVQAQVOLVETGGGLVRAGGSLRLSCAASGRTAD
IYNIGWFRQAPGKEREFVAAITWIGRTPYYADAVKGRFAFSTDSAKNTV
SLQMDNLKPEDTGVYYCNAAHYLEGNTDYYWGQGTQVTVSSAAATTTPA
PRPPTPAPTIASQPLSLRPEACRPAAGGAVHTRGLDFACDIYIWAPLAG
TCGVLLLSLVITLYCKRGRKKLLYIFKQPFMRPVQTTQEEDGCSCRFPE
EEEGGCELRVKFSRSADAPAYQQGQNQLYNELNLGRREEYDVLDKRRGR
DPEMGGKPRRKNPQEGLYNELQKDKMAEAYSEIGMKGERRRGKGHDGLY
QGLSTATKDTYDALHMQALPPR

CAR 7

An exemplary CAR construct comprising one or more single domain antibodies, or antigen-binding fragments thereof, described herein comprises a CD33 binding domain as comprised in SEQ ID NO: 9, a CD8a transmembrane domain, a CD8a hinge domain, a CD137 (4-1BB) co-stimulatory domain, and a CD3ζ intracellular signaling domain.

In some embodiments, a CAR comprises an amino acid sequence shown in SEQ ID NO: 92, or an amino acid sequence that is at least 70%, at least 75%, at least 80%, at least 85%, at least 90%, at least 95%, or at least 99% identical to the amino acid sequence shown in SEQ ID NO: 92.

[SEQ ID NO: 92]
MELGLSWVVLAALLQGVQAQVQLVQPGGSLRLFCVASEEFFSIYAMGWY
RQAPGKQHEMVARFTRDGKITYADSAKGRFTITRDAKNTLNLQMNGLIP
EDTAVYYCNINHYWGQGTQVTVSSAAATTTPAPRPPTPAPTIASQPLSL
RPEACRPAAGGAVHTRGLDFACDIYIWAPLAGTCGVLLLSLVITLYCKR
GRKKLLYIFKQPFMRPVQTTQEEDGCSCRFPEEEEGGCELRVKFSRSAD
APAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQ
ALPPR

CAR 8

An exemplary CAR construct comprising one or more single domain antibodies, or antigen-binding fragments thereof, described herein comprises a CD33 binding domain as comprised in SEQ ID NO:17, a CD8a transmembrane domain, a CD8a hinge domain, a CD137 (4-1BB) co-stimulatory domain, and a CD3ζ intracellular signaling domain.

In some embodiments, a CAR comprises an amino acid sequence shown in SEQ ID NO: 93, or an amino acid sequence that is at least 70%, at least 75%, at least 80%, at least 85%, at least 90%, at least 95%, or at least 99% identical to the amino acid sequence shown in SEQ ID NO: 93.

[SEQ ID NO: 93]
MELGLSWVVLAALLQGVQADVQLVESGGGLVQPGGSLRLSCSVSGNIDR
FYAMGWYRQAPGKQRELVAQLTNNEITTYGDSVEGQFSISGDFDANTVY
LQMDSLKPEDTAVYYCHAHVTTTRWSQDYYWGQGTRVTVSSAAATTTPA
PRPPTPAPTIASQPLSLRPEACRPAAGGAVHTRGLDFACDIYIWAPLAG
TCGVLLLSLVITLYCKRGRKKLLYIFKQPFMRPVQTTQEEDGCSCRFPE
EEEGGCELRVKFSRSADAPAYQQGQNQLYNELNLGRREEYDVLDKRRGR
DPEMGGKPRRKNPQEGLYNELQKDKMAEAYSEIGMKGERRRGKGHDGLY
QGLSTATKDTYDALHMQALPPR

Any of the fusion proteins, such as any of the CARs described herein may be expressed in a cell and thereby presented on the surface of the cell. In some embodiments, the cell may be an immune cell, such as a T cell (i.e., T lymphocyte) or an NK cell. A T cell lymphocyte can be any T cell, such as a cultured T cell, e.g., a primary T cell, or a T cell from a cultured T cell line, e.g., TIB-153™, Jurkat, SupT1, etc., or a T cell obtained from a mammal.

### Nucleotide Sequences and Expression

The present disclosure includes nucleotide sequences encoding any one or more anti-CD33 antibodies described herein (e.g., a VHH described herein), or portion thereof (e.g., one or more CDRs described herein), and/or one or more fusion proteins described herein. In various instances, such nucleotide sequences may be present in a vector, such as an expression vector. In various instances such nucleotides may be present in the genome of a cell, e.g., a cell of a subject in need of treatment or a cell for production of an antibody, e.g. a mammalian cell for production of an antibody.

In some embodiments, any of the antibodies described herein are encoded by a polynucleotide comprised in a vector, e.g., a viral vector. Optionally, a polynucleotide encoding a polypeptide as described herein can be codon-optimized to enhance expression or stability. Codon optimization may be performed according to any standard methods known in the art. In some embodiments, expression of the polypeptide can be driven by a constitutively expressed promoter or an inducibly expressed promoter. In some embodiments, an antibody as described herein includes a signal peptide. Signal peptides can be derived from any protein that has an extracellular domain or is secreted. An antibody as described herein may include any signal peptides known in the art.

Retroviruses, such as lentiviruses, provide a convenient platform for delivery of nucleic acid sequences encoding a gene, or chimeric gene of interest. A selected nucleic acid sequence can be inserted into a vector and packaged in retroviral particles using techniques known in the art. The recombinant virus can then be isolated and delivered to cells, e.g. in vitro or ex vivo. Retroviral systems are well known in the art and are described in, for example, U.S. Pat. No. 5,219,740; Kurth and Bannert (2010) “Retroviruses: Molecular Biology, Genomics and Pathogenesis” Calster Academic Press (ISBN:978-1-90455-55-4); and Hu and Pathak Pharmacological Reviews 2000 52:493-512; which are incorporated by reference herein in their entirety. In some embodiments, an antibody described herein is expressed in a mammalian cell via transfection or electroporation of an expression vector comprising nucleic acid encoding the antibody. Transfection or electroporation methods are known in the art.

In another aspect, the disclosure relates to a cell, e.g., a mammalian cell, comprising any of the antibodies described herein; or a nucleic acid encoding any of the antibodies described herein. In one embodiment, the cell comprises an antibody described herein, or a nucleic acid encoding such an antibody described herein. The cell or tissue, e.g., mammalian cell or tissue, can be of human, primate, hamster, rabbit, rodent, cow, pig, sheep, horse, goat, dog or cat origin. In some embodiments, any other mammalian cell may be used. In some embodiments, the mammalian cell is human.

Efficient expression of an antibody described herein can be assessed using standard assays that detect the mRNA, DNA, or a gene product of the nucleic acid encoding the antibody, such as RT-PCR, FACS, northern blotting, western blotting, ELISA, or immunohistochemistry. In some embodiments, the antibody described herein is encoded by recombinant nucleic acid sequence.

### CD33

CD33, also known as Siglec (Sialic-acid-binding immunoglobulin-like lectin) plays a role in mediating cell-cell interactions and in maintaining immune cells in a resting state. CD33 preferentially recognizes and binds alpha-2,3- and more avidly alpha-2,6-linked sialic acid-bearing glycans and upon engagement of ligands such as C1q or syalylated glycoproteins, two immunoreceptor tyrosine-based inhibitory motifs (ITIMs) located in the cytoplasmic tail of CD33 are phosphorylated by Src-like kinases such as LCK. These phosphorylations provide docking sites for the recruitment and activation of protein-tyrosine phosphatases PTPN6/SHP-1 and PTPN11/SHP-2. In turn, these phosphatases are thought to regulate downstream pathways through dephosphorylation of signaling molecules. One of the repressive effect of CD33 on monocyte activation requires phosphoinositide 3-kinase/PI3 K.

CD33 is expressed by myeloid stem cells (CFU-GEMM, CFU-GM, CFU-G, and E-BFU), myeloblasts and monoblasts, monocytes/macrophages, granulocyte precursors (with decreasing expression with maturation), and mast cells. Mature granulocytes may show a very low level of CD33 expression. CD33 can be aberrantly expressed on some cases of plasma-cell myeloma. CD33 is not expressed in erythrocytes, platelets, B cells, T cells, or NK cells. CD33 is an excellent myeloid marker and is commonly used for the diagnosis of acute myeloid leukemia (AML). However, approximately 10-20% of B-lymphoblastic or T-lymphoblastic leukemia/lymphomas may aberrantly express CD33 (see Naeim et al., Atlas of Hematopathology (Second Edition), 2018).

### CD33-Associated Disorders

Any of the antibodies and/or fusion proteins of the disclosure can be used, e.g., to detect and/or treat CD33-associated disorders, i.e., diseases correlated with elevated or reduced cell surface expression of CD33 as compared to CD33 expression in a standard control (e.g., a normal, non-disease, non-cancer cell). In some embodiments, a CD33-associated disorder comprises a blood malignancy or neoplasm. In some embodiments, a blood malignancy or neoplasm comprises myelodysplastic syndrome (MDS), acute myeloid leukemia (AML), or multiple myeloma (MM). In some embodiments, a CD33-associated disorder comprises AML.

Acute myeloid leukemia (AML) is a cancer of the bone marrow that needs more effective therapies. According to the National Cancer Institute, more than 60,000 people in the U.S. have AML, and less than 30% of patients survive five years following diagnosis. AML cells can be characterized and distinguished from other cells by detecting cell surface marker expression. AML cells can be CD33+(though some are CD33−), CLL-1+, CD45+, and CDw52+. AML is characterized as a heterogeneous, clonal, neoplastic disease that originates from transformed cells that have progressively acquired critical genetic changes that disrupt key differentiation and growth-regulatory pathways. (Dohner et al., NEJM, (2015) 373:1136).

CD33 is also frequently expressed in cases of myelodysplastic syndrome and chronic myelomonocytic leukemia with elevated blast count (see Sanford et al. Leuk Lymphoma (2016) 57(8): 1965-1968) and also expressed on plasma cells of significant number of myeloma patients, indicating that it may represent a therapeutic target for multiple myeloma (see Robillard et al. Leukemia (2005) 19(11):2021-2).

In some embodiments, the hematopoietic malignancy or hematological disorder associated with CD123 is a precancerous condition such as a myelodysplasia, a myelodysplastic syndrome or a preleukemia. Myelodysplastic syndromes (MDS) are hematological medical conditions characterized by disorderly and ineffective hematopoiesis, or blood production. Thus, the number and quality of blood-forming cells decline irreversibly. Some patients with MDS can develop severe anemia, while others are asymptomatic. The classification scheme for MDS is known in the art, with criteria designating the ratio or frequency of particular blood cell types, e.g., myeloblasts, monocytes, and red cell precursors. MDS includes refractory anemia, refractory anemia with ring sideroblasts, refractory anemia with excess blasts, refractory anemia with excess blasts in transformation, chronic myelomonocytic leukemia (CML). In some embodiments, MDS can progress to an acute myeloid leukemia (AML).

In various instances, an antibody and/or fusion protein and/or cells expressing any of the foregoing described herein treats, alleviates, reduces the prevalence of, reduces the frequency of, or reduces the level or amount of one or more symptoms or biomarkers of a CD33-associated disorder (e.g., AML, MDS, MM). Specific symptoms and progression of symptoms vary among subjects. Thus, in some embodiments, an antibody and/or fusion protein described herein is administered to a subject in need thereof, e.g., a subject having a CD33-associated disorder (e.g., AML, MDS, MM). In some embodiments, administration of any of the antibodies and/or fusion proteins described herein prevents cancer or hematopoietic malignancy or pre-malignancy, including reducing one or more symptoms and/or delaying the progression of the disease.

In various instances, administration of an antibody and/or fusion protein described herein results in a decrease in the prevalence, frequency, level, and/or amount of one or more symptoms or biomarkers of a CD33-associated disorder, e.g., a decrease of at least 3%, 4%, 5%, 6%, 7%, 8%, 9%, 10%, 15%, 20%, 25%, 30%, 35%, 40%, 45%, 50%, 60%, 70%, 80%, 90%, 95%, 99%, or 100% of one or more symptoms or biomarkers as compared to a prior measurement in the subject or to a reference value.

In some embodiments, an effective dose of an antibody and/or fusion protein as described herein may be, e.g., less than 1,000 mg/dose, e.g., less than 900 mg/dose, 800 mg/dose, 700 mg/dose, 600 mg/dose, 500 mg/dose, 550 mg/dose, 400 mg/dose, 350 mg/dose, 300 mg/dose, 200 mg/dose, 100 mg/dose, 50 mg/dose, 25 mg/dose, or less. Alternatively or in combination with a dosage as disclosed herein, an antibody and/or fusion protein as described herein may be effectively or usefully administered at a frequency that is less than once per week, e.g., less than once every week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months, 7 months, 8 months, 9 months, 10 months, 11 months, or year.

In some embodiments, an effective dose of a cell (e.g., immune cells) expressing fusion protein, such as a CAR as described herein may be for example, in the range of one million to 100 billion cells; however, amounts below or above this exemplary range are also within the scope of the present disclosure. For example, the daily dose of cells can be about 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), preferably about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), more preferably about 100 million cells to about 50 billion cells (e.g., about 120 million cells, about 350 million cells, about 350 million cells, about 450 million cells, about 650 million cells, about 800 million cells, about 900 million cells, about 3 billion cells, about 30 billion cells, about 45 billion cells, or a range defined by any two of the foregoing values).

In some embodiments, an antibody and/or fusion protein described herein can be used in a number of diagnostic and/or therapeutic applications. For example, detectably-labeled versions of antibodies as described herein can be used in assays to detect the presence or amount of CD33 in a sample (e.g., a biological sample). Antibodies and/or fusion proteins described herein can be used in in vitro assays for studying inhibition of CD33 activity. In some embodiments, an antibody and/or fusion protein described herein can be used as a positive control in an assay designed to identify additional novel compounds that inhibit CD33 or otherwise are useful for treating a CD33-associated disorder.

Antibodies and/or fusion proteins described herein may be used in monitoring a subject, e.g., a subject having, suspected of having, at risk of developing, or under treatment for one or more CD33-associated disorders. Monitoring may include determining the amount or activity of CD33 in a subject, e.g., in the serum of a subject. In some embodiments, the evaluation is performed at least one (1) hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration of an antibody and/or fusion protein as described herein. The subject can be evaluated in one or more of the following periods: prior to beginning of treatment; during the treatment; or after one or more elements of the treatment have been administered. Evaluation can include evaluating the need for further treatment, e.g., evaluating whether a dosage, frequency of administration, or duration of treatment should be altered. It can also include evaluating the need to add or drop a selected therapeutic modality, e.g., adding or dropping any of the treatments for a CD33-associated disorder described herein.

### Measuring Interactions of Antibodies and CD33

The binding properties of an antibody described herein to CD33 can be measured by methods known in the art, e.g., one of the following methods: BIACORE analysis, Enzyme Linked Immunosorbent Assay (ELISA), x-ray crystallography, sequence analysis and scanning mutagenesis. The binding interaction of an antibody and CD33 can be analyzed using surface plasmon resonance (SPR). SPR or Biomolecular Interaction Analysis (BIA) detects bio-specific interactions in real time, without labeling any of the interactants. Changes in the mass at the binding surface (indicative of a binding event) of the BIA chip result in alterations of the refractive index of light near the surface. The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden). Additionally, a KinExA® (Kinetic Exclusion Assay) assay, available from Sapidyne Instruments (Boise, Id.) can also be used.

Information from SPR can be used to provide an accurate and quantitative measure of the equilibrium dissociation constant (K_{D}), and kinetic parameters, including Kₒₙ and K_{off}, for the binding of an antibody to CD33. Such data can be used to compare different molecules. Information from SPR can also be used to develop structure-activity relationships (SAR). Variant amino acids at given positions can be identified that correlate with particular binding parameters, e.g., high affinity.

In certain embodiments, an antibody described herein exhibits high affinity for binding CD33. In various embodiments, K_{D} of an antibody as described herein for CD33 is less than about 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, or 10⁻¹⁵ M. In certain instances, K_{D} of an antibody as described herein for CD33 is between 0.001 and 1 nM, e.g., 0.001 nM, 0.005 nM, 0.01 nM, 0.05 nM, 0.1 nM, 0.5 nM, or 1 nM.

In some embodiments, an antibody described herein binds to a specific epitope of CD33, e.g., comprising one or more specific amino acids of CD33. Without wishing to be bound by theory, disruption of the availability of the specific amino acids of a CD33 epitope of an antibody described herein (e.g., by mutagenesis or due to binding by a competing antibody) may decrease or eliminate binding of the antibody. In therapeutic applications or product manufacture where multiple anti-CD33 antibody to CD33 interactions are desired, it may be important to select or design anti-CD33 antibodies to target non-competing CD33 epitopes to avoid interfering with one another. In some embodiments, an anti-CD33 antibody described herein specifically binds a CD33 epitope comprising 1, 2, 3, 4, 5, or all of amino acids W22, H45, Y49, Y50, R98, and R122 (e.g., all of W22, H45, Y49, Y50, R98, and R122). In some embodiments, an anti-CD33 antibody described herein specifically binds a CD33 epitope comprising 1, 2, or all of amino acids Y49, Y50, and L78 (e.g., all of Y49, Y50, and L78). In some embodiments, an anti-CD33 antibody described herein specifically binds a CD33 epitope comprising 1, 2, 3, or all of amino acids K52, L78, R98, and R122 (e.g., all of K52, L78, R98, and R122). In some embodiments, an anti-CD33 antibody described herein specifically binds a CD33 epitope comprising 1, 2, 3, or all of amino acids Y49, Y50, K52, and R98 (e.g., all of Y49, Y50, K52, and R98). In some embodiments, an anti-CD33 antibody described herein specifically binds a CD33 epitope comprising 1, 2, 3, or all of amino acids N20, H45, Y49, and Y50 (e.g., all of N20, H45, Y49, and Y50). In some embodiments, an anti-CD33 antibody described herein specifically binds a CD33 epitope comprising W22. In some embodiments, an anti-CD33 antibody described herein specifically binds a CD33 epitope comprising 1, 2, or all of amino acids Y49, Y50, and N99 (e.g., all of Y49, Y50, and N99). In some embodiments, an anti-CD33 antibody described herein specifically binds a CD33 epitope comprising 1, 2, 3, 4, or all of amino acids H45, Y49, Y50, K52, and R122 (e.g., all of H45, Y49, Y50, K52, and R122).

### Methods of Treatment

In some embodiments, one or more anti-CD33 antibodies described herein are used in a method of treating one or more disorders described herein, e.g., one or more diseases or disorders associated with CD33 expression. Diseases or disorders associated with CD33 expression include, for example, certain blood malignancies and neoplasms, e.g., MDS, AML, and MM. In some embodiments, the method comprises administering to a subject in need thereof a therapeutically effective amount of an antibody, or antigen-binding fragment thereof, described herein, or a cell expressing any of the foregoing. In some embodiments, one or more anti-CD33 antibodies described herein, including fusion proteins comprising any of the anti-CD33 antibodies, are used in a method of treating diseases or disorders associated with CD33 expression. In some embodiments, one or more anti-CD33 antibodies described herein are used in a method of treating neoplastic diseases and malignancies of the blood that are associated with CD33 expression. In some embodiments, one or more anti-CD33 antibodies described herein are used in a method of treating MDS, AML, or MM.

### Combination Therapy

In some embodiments, an anti-CD33 antibody described herein is administered in combination with one or more additional therapeutic agents, such as a chemotherapeutic agent or an oncolytic therapeutic agent. “Combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. Administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

As used herein, the term “chemotherapeutic agent” or “oncolytic therapeutic agent” (e.g., anti-cancer drug, e.g., anti-cancer therapy, e.g., immune cell therapy) has its art-understood meaning referring to one or more pro-apoptotic, cytostatic and/or cytotoxic agents, and/or hormonal agents, for example, specifically including agents utilized and/or recommended for use in treating one or more diseases, disorders or conditions associated with undesirable cell proliferation. In some embodiments, a chemotherapeutic agent and/or oncolytic therapeutic agent may be or comprise platinum compounds (e.g., cisplatin, carboplatin, and oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, and bendamustine), antitumor antibiotics (e.g., daunorubicin, doxorubicin, idarubicin, epirubicin, mitoxantrone, bleomycin, mytomycin C, plicamycin, and dactinomycin), taxanes (e.g., paclitaxel and docetaxel), antimetabolites (e.g., 5-fluorouracil, cytarabine, premetrexed, thioguanine, floxuridine, capecitabine, and methotrexate), nucleoside analogues (e.g., fludarabine, clofarabine, cladribine, pentostatin, and nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vicristine, vindesine, vinorelbine, and vinblastine), tyrosine kinase inhibitors (e.g., imatinib, dasatinib, nilotinib, sorafenib, and sunitinib), nitrosoureas (e.g., carmustine, fotemustine, and lomustine), hexamethylmelamine, mitotane, angiogenesis inhibitors (e.g., thalidomide and lenalidomide), steroids (e.g., prednisone, dexamethasone, and prednisolone), hormonal agents (e.g., tamoxifen, raloxifene, leuprolide, bicaluatmide, granisetron, and flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, and oxaprozin), selective cyclooxygenase-2 (COX-2) inhibitors, or any combination thereof.

In certain embodiments, chemotherapeutic agents and/or oncolytic therapeutic agents for anti-cancer treatment comprise biological agents such as tumor-infiltrating lymphocytes, CAR T-cells, antibodies, antigens, therapeutic vaccines (e.g., made from a patient's own tumor cells or other substances such as antigens that are produced by certain tumors), immune-modulating agents (e.g., cytokines, e.g., immunomodulatory drugs or biological response modifiers), checkpoint inhibitors or other immunologic agents. In certain embodiments, immunologic agents include immunoglobins, immunostimulants (e.g., bacterial vaccines, colony stimulating factors, interferons, interleukins, therapeutic vaccines, vaccine combinations, viral vaccines) and/or immunosuppressive agents (e.g., calcineurin inhibitors, interleukin inhibitors, TNF alpha inhibitors). In certain embodiments, hormonal agents include agents for anti-androgen therapy (e.g., Ketoconazole, ABiraterone, TAK-700, TOK-OO1, Bicalutamide, Nilutamide, Flutamide, Enzalutamide, ARN-509).

Additional chemotherapeutic agents and/or oncolytic therapeutic agents include immune checkpoint therapeutics (e.g., pembrolizumab, nivolumab, ipilimumab, atezolizumab, avelumab, durvalumab, tremelimumab, or cemiplimab), other monoclonal antibodies (e.g., rituximab, cetuximab, panetumumab, tositumomab, trastuzumab, alemtuzumab, gemtuzumab ozogamicin, bevacizumab, catumaxomab, denosumab, obinutuzumab, ofatumumab, ramucirumab, pertuzumab, nimotuzumab, lambrolizumab, pidilizumab, siltuximab, BMS-936559, RG7446/MPDL3280A, MEDI4736), antibody-drug conjugates (e.g., brentuximab vedotin (ADCETRIS®, Seattle Genetics); ado-trastuzumab emtansine (KADCYLA®, Roche); Gemtuzumab ozogamicin (Wyeth); CMC-544; SAR3419; CDX-011; PSMA-ADC; BT-062; and IMGN901 (see, e.g., Sassoon et al., Methods Mol. Biol. 1045:1-27 (2013); Bouchard et al., Bioorganic Med. Chem. Lett. 24: 5357-5363 (2014)), or any combination thereof.

In some embodiments, combined administration of an anti-CD33 antibody and an additional therapeutic agent results in an improvement in cancer to an extent that is greater than one produced by either the anti-CD33 antibody or the additional therapeutic agent alone. The difference between the combined effect and the effect of each agent alone can be a statistically significant difference. In some embodiments, the combined effect can be a synergistic effect. In some embodiments, combined administration of an anti-CD33 antibody and an additional therapeutic agent allows administration of the additional therapeutic agent at a reduced dose, at a reduced number of doses, and/or at a reduced frequency of dosage compared to a standard dosing regimen, e.g., an approved dosing regimen for the additional therapeutic agent.

In some embodiments, treatment methods described herein are performed on subjects for whom other treatments of the medical condition have failed or have had less success in treatment through other means. Additionally, the treatment methods described herein can be performed in conjunction with one or more additional treatments of the medical condition. For instance, the method can comprise administering a cancer regimen, e.g., non-myeloablative chemotherapy, surgery, hormone therapy, and/or radiation, prior to, substantially simultaneously with, or after the administration of an anti-CD33 antibody described herein, or composition thereof.

Aspects of the present disclosure involve administration of hematopoietic cells that are genetically modified to have reduced or eliminated expression of CD33, e.g., in the context of treating a subject in need of such hematopoietic stem cells, which may include, for example, a subject having a hematologic malignancy, such as, e.g., AML, or premalignancy, such as, e.g., MDS, and undergoing an immunotherapy regimen targeting CD33, e.g., a CD33-antibody-drug conjugate or a CD33 CAR-T or CAR-NK therapy. Such treatment regimen can involve, for example, the following steps: (1) administering a therapeutically effective amount of any of the anti-CD33 antibodies, of CD33 bringing fragments thereof, including fusion proteins, such as e.g., CARs, and cells expressing any of the foregoing, e.g., CAR-T or CAR-NK cells, as described herein or otherwise apparent to the skilled artisan based on the present disclosure; and (2) administering (e.g., infusing or reinfusing) the patient with hematopoietic stem cells, either autologous or allogeneic, where the hematopoietic cells have reduced expression or eliminated expression of CD33. In some embodiments, the hematopoietic cells are genetically modified to have reduced expression of CD33. In some embodiments, the hematopoietic cells are genetically modified to have eliminated expression of CD33. In some embodiments, the hematopoietic cells are genetically modified to have reduced or eliminated expression of a CD33 epitope bound by an antibody or a CD33-binding fragment thereof, as disclosed herein.

### Formulations and Administration

In various embodiments, an antibody described herein can be incorporated into a pharmaceutical composition. Such a pharmaceutical composition can be useful, e.g., for the prevention and/or treatment of diseases, e.g., cancers, such as AML, MDS, MM. Pharmaceutical compositions can be formulated by methods known to those skilled in the art (such as described in Remington's Pharmaceutical Sciences, 17th edition, ed. Alfonso R. Gennaro, Mack Publishing Company, Easton, Pa. (1985)).

In some embodiments, a pharmaceutical composition can be formulated to include a pharmaceutically acceptable carrier or excipient. Examples of pharmaceutically acceptable carriers include, without limitation, any and all solvents, dispersion media, coatings, antibacterial and antifungal agents, isotonic and absorption delaying agents, and the like that are physiologically compatible. Compositions of the present invention can include a pharmaceutically acceptable salt, e.g., an acid addition salt or a base addition salt.

In some embodiments, a composition including an antibody as described herein, e.g., a sterile formulation for injection, can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, such as, for example, an alcohol such as ethanol and/or a polyalcohol such as propylene glycol or polyethylene glycol, and/or a nonionic surfactant such as polysorbate 80™ or HCO-50.

As disclosed herein, a pharmaceutical composition may be in any form known in the art. Such forms include, e.g., liquid, semi-solid and solid dosage forms, such as liquid solutions (e.g., injectable and infusible solutions), dispersions or suspensions, tablets, pills, powders, liposomes and suppositories.

Selection or use of any particular form may depend, in part, on the intended mode of administration and therapeutic application. For example, compositions containing a composition intended for systemic or local delivery can be in the form of injectable or infusible solutions. Accordingly, compositions can be formulated for administration by a parenteral mode (e.g., intravenous, subcutaneous, intraperitoneal, or intramuscular injection). As used herein, parenteral administration refers to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion.

Route of administration can be parenteral, for example, administration by injection, transnasal administration, transpulmonary administration, or transcutaneous administration. Administration can be systemic or local by intravenous injection, intramuscular injection, intraperitoneal injection, or subcutaneous injection.

In some embodiments, a pharmaceutical composition of the present invention can be formulated as a solution, microemulsion, dispersion, liposome, or other ordered structure suitable for stable storage at high concentration. Sterile injectable solutions can be prepared by incorporating a composition described herein in the required amount in an appropriate solvent with one or a combination of ingredients enumerated above, as required, followed by filter sterilization. Generally, dispersions are prepared by incorporating a composition described herein into a sterile vehicle that contains a basic dispersion medium and the required other ingredients from those enumerated above. In the case of sterile powders for the preparation of sterile injectable solutions, methods for preparation include vacuum drying and freeze-drying that yield a powder of a composition described herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

A pharmaceutical composition can be administered parenterally in the form of an injectable formulation comprising a sterile solution or suspension in water or another pharmaceutically acceptable liquid. For example, the pharmaceutical composition can be formulated by suitably combining the therapeutic molecule with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of active ingredient included in a pharmaceutical preparation is such that a suitable dose within the designated range is provided. Non-limiting examples of oily liquid include sesame oil and soybean oil, and may be combined with benzyl benzoate or benzyl alcohol as a solubilizing agent. Other items that may be included are a buffer such as a phosphate buffer, or sodium acetate buffer, a soothing agent such as procaine hydrochloride, a stabilizer such as benzyl alcohol or phenol, and an antioxidant. A formulated injection can be packaged in a suitable ampule.

In various embodiments, subcutaneous administration can be accomplished by means of a device, such as a syringe, a prefilled syringe, an auto-injector (e.g., disposable or reusable), a pen injector, a patch injector, a wearable injector, an ambulatory syringe infusion pump with subcutaneous infusion sets, or other device for combining with antibody drug for subcutaneous injection.

An injection system of the present disclosure may employ a delivery pen as described in U.S. Pat. No. 5,308,341. Pen devices, most commonly used for self-delivery of insulin to patients with diabetes, are well known in the art. Such devices can comprise at least one injection needle (e.g., a 31 gauge needle of about 5 to 8 mm in length), are typically pre-filled with one or more therapeutic unit doses of a therapeutic solution, and are useful for rapidly delivering solution to a subject with as little pain as possible. One medication delivery pen includes a vial holder into which a vial of a therapeutic or other medication may be received. The pen may be an entirely mechanical device or it may be combined with electronic circuitry to accurately set and/or indicate the dosage of medication that is injected into the user. See, e.g., U.S. Pat. No. 6,192,891. In some embodiments, the needle of the pen device is disposable and the kits include one or more disposable replacement needles. Pen devices suitable for delivery of any one of the presently featured compositions are also described in, e.g., U.S. Pat. Nos. 6,277,099; 6,200,296; and 6,146,361, the disclosures of each of which are incorporated herein by reference in their entirety. A microneedle-based pen device is described in, e.g., U.S. Pat. No. 7,556,615, the disclosure of which is incorporated herein by reference in its entirety. See also the Precision Pen Injector (PPI) device, MOLLY™, manufactured by Scandinavian Health Ltd.

In some embodiments, a composition described herein can be therapeutically delivered to a subject by way of local administration. As used herein, “local administration” or “local delivery,” can refer to delivery that does not rely upon transport of the composition or agent to its intended target tissue or site via the vascular system. For example, the composition may be delivered by injection or implantation of the composition or agent or by injection or implantation of a device containing the composition or agent. In certain embodiments, following local administration in the vicinity of a target tissue or site, the composition or agent, or one or more components thereof, may diffuse to an intended target tissue or site that is not the site of administration.

In some embodiments, a composition can be formulated for storage at a temperature below 0° C. (e.g., −20° C. or −80° C.). In some embodiments, the composition can be formulated for storage for up to 2 years (e.g., one month, two months, three months, four months, five months, six months, seven months, eight months, nine months, 10 months, 11 months, 1 year, 1½ years, or 2 years) at 2-8° C. (e.g., 4° C.). Thus, in some embodiments, the compositions described herein are stable in storage for at least 1 year at 2-8° C. (e.g., 4° C.).

In some embodiments, a pharmaceutical composition can be formulated as a solution. In some embodiments, a composition can be formulated, for example, as a buffered solution at a concentration suitable for storage at 2-8° C. (e.g., 4° C.).

Compositions including one or more antibodies as described herein can be formulated in immunoliposome compositions. Such formulations can be prepared by methods known in the art. Liposomes with enhanced circulation time are disclosed in, e.g., U.S. Pat. No. 5,013,556.

In certain embodiments, compositions can be formulated with a carrier that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are known in the art. See, e.g., J. R. Robinson (1978) “Sustained and Controlled Release Drug Delivery Systems,” Marcel Dekker, Inc., New York.

In some embodiments, administration of an antibody as described herein is achieved by administering to a subject a nucleic acid encoding the antibody. Nucleic acids encoding a therapeutic antibody described herein can be incorporated into a gene construct to be used as a part of a gene therapy protocol to deliver nucleic acids that can be used to express and produce antibody within cells. Expression constructs of such components may be administered in any therapeutically effective carrier, e.g. any formulation or composition capable of effectively delivering the component gene to cells in vivo. Approaches include insertion of the subject gene in viral vectors including recombinant retroviruses, adenovirus, adeno-associated virus, lentivirus, and herpes simplex virus-1 (HSV-1), or recombinant bacterial or eukaryotic plasmids. Viral vectors can transfect cells directly; plasmid DNA can be delivered with the help of, for example, cationic liposomes (lipofectin) or derivatized, polylysine conjugates, gramicidin S, artificial viral envelopes or other such intracellular carriers, as well as direct injection of the gene construct or CaPO₄ precipitation (see, e.g., WO04/060407). Examples of suitable retroviruses include pLJ, pZIP, pWE and pEM which are known to those skilled in the art (see, e.g., Eglitis et al. (1985) Science 230:1395-1398; Danos and Mulligan (1988) Proc Natl Acad Sci USA 85:6460-6464; Wilson et al. (1988) Proc Natl Acad Sci USA 85:3014-3018; Armentano et al. (1990) Proc Natl Acad Sci USA 87:6141-6145; Huber et al. (1991) Proc Natl Acad Sci USA 88:8039-8043; Ferry et al. (1991) Proc Natl Acad Sci USA 88:8377-8381; Chowdhury et al. (1991) Science 254:1802-1805; van Beusechem et al. (1992) Proc Natl Acad Sci USA 89:7640-7644; Kay et al. (1992) Human Gene Therapy 3:641-647; Dai et al. (1992) Proc Natl Acad Sci USA 89:10892-10895; Hwu et al. (1993) J Immunol 150:4104-4115; U.S. Pat. Nos. 4,868,116 and 4,980,286; and PCT Publication Nos. WO89/07136, WO89/02468, WO89/05345, and WO92/07573). Another viral gene delivery system utilizes adenovirus-derived vectors (see, e.g., Berkner et al. (1988) BioTechniques 6:616; Rosenfeld et al. (1991) Science 252:431-434; and Rosenfeld et al. (1992) Cell 68:143-155). Suitable adenoviral vectors derived from the adenovirus strain Ad type 5 d1324 or other strains of adenovirus (e.g., Ad2, Ad3, Ad7, etc.) are known to those skilled in the art. Yet another viral vector system useful for delivery of the subject gene is the adeno-associated virus (AAV). See, e.g., Flotte et al. (1992) Am J Respir Cell Mol Biol 7:349-356; Samulski et al. (1989) J Virol 63:3822-3828; and McLaughlin et al. (1989) J Virol 62:1963-1973.

In some embodiments, the compositions provided herein are present in unit dosage form, which unit dosage form can be suitable for self-administration. Such a unit dosage form may be provided within a container, typically, for example, a vial, cartridge, prefilled syringe or disposable pen. A doser such as the doser device described in U.S. Pat. No. 6,302,855, may also be used, for example, with an injection system as described herein.

A suitable dose of a composition described herein, which dose is capable of treating or preventing a disorder in a subject, can depend on a variety of factors including, e.g., the age, sex, and weight of a subject to be treated and the particular inhibitor compound used. For example, a different dose of one composition including an antibody as described herein may be required to treat a subject with a cancer (e.g., AML) as compared to the dose of a different formulation of that antibody. Other factors affecting the dose administered to the subject include, e.g., the type or severity of the disorder. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject can also be adjusted based upon the judgment of the treating medical practitioner.

A composition described herein can be administered as a fixed dose, or in a milligram per kilogram (mg/kg) dose. In some embodiments, the dose can also be chosen to reduce or avoid production of antibodies or other host immune responses against one or more of the antigen-binding molecules in the composition. Exemplary dosages of an antibody, such as a composition described herein, include, e.g., 0.0001 to 100 mg/kg, 0.01 to 5 mg/kg, 1-1000 mg/kg, 1-100 mg/kg, 0.5-50 mg/kg, 0.1-100 mg/kg, 0.5-25 mg/kg, 1-20 mg/kg, and 1-10 mg/kg of the subject body weight. For example dosages can be 0.1 mg/kg, 0.3 mg/kg, 0.5 mg/kg, 1.0 mg/kg, 2.0 mg/kg, 3.0 mg/kg, 4.0 mg/kg, 5.0 mg/kg, 10 mg/kg or 20 mg/kg body weight or within the range of 1-20 mg/kg body weight. An exemplary treatment regime entails administration once per week, once every two weeks, once every three weeks, once every four weeks, once a month, once every 3 months or once every three to 6 months, or with a short administration interval at the beginning (such as once per week to once every three weeks), and then an extended interval later (such as once a month to once every three to 6 months).

A pharmaceutical solution can include a therapeutically effective amount of a composition described herein. Such effective amounts can be readily determined by one of ordinary skill in the art based, in part, on the effect of the administered composition, or the combinatorial effect of the composition and one or more additional active agents, if more than one agent is used. A therapeutically effective amount of a composition described herein can also vary according to factors such as the disease state, age, sex, and weight of the individual, and the ability of the composition (and one or more additional active agents) to elicit a desired response in the individual, e.g., amelioration of at least one condition parameter, e.g., amelioration of at least one symptom of a cancer (e.g., AML). For example, a therapeutically effective amount of a composition described herein can inhibit (lessen the severity of or eliminate the occurrence of) and/or prevent a particular disorder, and/or any one of the symptoms of the particular disorder known in the art or described herein. A therapeutically effective amount is also one in which any toxic or detrimental effects of the composition are outweighed by the therapeutically beneficial effects.

Suitable human doses of any of the compositions described herein can further be evaluated in, e.g., Phase I dose escalation studies. See, e.g., van Gurp et al. Am J Transplantation (2008) 8(8):1711-1718; Hanouska et al. Clin Cancer Res (2007) 13(2, part 1):523-531; and Hetherington et al. Antimicrobial Agents and Chemotherapy (2006) 50(10): 3499-3500.

Toxicity and therapeutic efficacy of compositions can be determined by known pharmaceutical procedures in cell cultures or experimental animals (e.g., animal models of any of the cancers described herein). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. A composition described herein that exhibits a high therapeutic index is preferred. While compositions that exhibit toxic side effects may be used, care should be taken to design a delivery system that targets such compounds to the site of affected tissue and to minimize potential damage to normal cells and, thereby, reduce side effects.

Those of skill in the art will appreciate that data obtained from cell culture assays and animal studies can be used in formulating a range of dosage for use in humans. Appropriate dosages of compositions described herein lie generally within a range of circulating concentrations of the compositions that include the ED₅₀ with little or no toxicity. The dosage may vary within this range depending upon the dosage form employed and the route of administration utilized. For a composition described herein, the therapeutically effective dose can be estimated initially from cell culture assays. A dose can be formulated in animal models to achieve a circulating plasma concentration range that includes the IC₅₀ (i.e., the concentration of the antibody which achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration (e.g., to the eye or a joint) is desired, cell culture or animal modeling can be used to determine a dose required to achieve a therapeutically effective concentration within the local site.

### NFAT-Responsive Reporter Systems

Aspects of the present disclosure relate to nucleic acid constructs comprising a minimal nuclear factor of activated T cells (NFAT)-responsive promoter, which may be used, for example, to assess the activity of chimeric antigen receptors (CARs) and activation of a cell (e.g., T cells) expressing the CARs comprising any of the anti-CD3 antibodies described herein. CAR activation sets in motion an intracellular pathway leading to T-cell activation and effector function of the T cell, which involves NFAT signaling and gene expression (see, e.g., Hogan, Cell Calcium. (2017)63:66-9). As used herein, the term “NFAT-responsive promoter” refers to a promoter region that is activated by NFAT signaling and promotes expression of a gene that is operably linked to the NFAT-responsive promoter upon activation. In some embodiments, the gene that is operably linked (under control of) the NFAT-responsive promoter encodes a reporter molecule.

Nuclear factor of activated T-cells (NFAT) is a family of transcription factors, include NFAT1-NFAT-5, that are involved regulating immune responses, including regulating interleukin-2 (IL-2 expression) as well as T cell differentiation and self-tolerance. See, e.g., Macian Nat. Rev. Immunol. (2005) 5: 472-484. NFAT transcription factors comprise two components: a cytoplasmic Rel domain protein (NFAT family member) and a nuclear component comprising various transcription factors (Chow, Molecular and Cellular Biology, 1999; 19(3):2300-7). NFAT1 and NFAT2 are predominantly expressed in peripheral T cells that produce IL-2 and NFAT binding sites are generally found upstream (5′) of NFAT-regulated genes, such as IL-2. See, e.g., Chow, Molecular and Cellular Biology, (1999) 19(3):2300-7; Rooney et al., Molecular and Cellular Biology, (1995) 15(11):6299-310; and Shaw et al., Journal of Immunology, (2010) 185(9):4972-5, the entire contents of which are incorporated herein by reference.

As will be understood by one of ordinary skill in the art, in eukaryotic cells, a promoter operably linked to a gene typically includes a core promoter adjacent and 5′ to the transcription start site of the gene (coding sequence). Further upstream (5′) of the core promoter may be cis-regulatory regions, such as transcription factor binding site(s).

In some embodiments, the NFAT-responsive promoter comprises a plurality of NFAT-binding sites. In some embodiments, the NFAT-responsive promoter comprises least 1, 2, 3, 4, 5, 6, 7, 8, 9, 10, 11, 12, 13, 14, 15, 16, 17, 18, 19, 20 or more NFAT binding sites. In some embodiments, the NFAT-responsive promoter comprises six NFAT binding sites. In some embodiments, each of the NFAT binding sites of a NFAT-responsive promoter may be the same NFAT binding site (e.g., bind the same type of NFAT transcription factor) or be different NFAT binding sites (e.g., bind different types of NFAT transcription factors). In some embodiments, each of the NFAT binding site comprises the same nucleotide sequence. In some embodiments, the NFAT binding sites comprise different nucleotide sequence.

An example of a NFAT binding site is provided by the nucleotide sequence provided by SEQ ID NO: 94:

(SEQ ID NO: 94)
5′-GGAGGAAAAACTGTTTCATACAGAAGGCGT-3′.

In some embodiments, at least one of the NFAT binding site comprises the nucleotide sequence of SEQ ID NO: 94. In some embodiments, each of the NFAT binding site comprises the nucleotide sequence of SEQ ID NO: 94.

Each of the NFAT binding sites are located immediately adjacent to one another (e.g., in tandem without any additional nucleotides between the NFAT binding sites). Alternatively, one or more additional nucleotides may be present between two or more of the NFAT binding sites.

In some embodiments, the NFAT-responsive promoter comprises an IL-2 promoter, or portion thereof. In some embodiments, the NFAT-responsive promoter comprises a minimal IL-2 promoter. In some embodiments, the NFAT-responsive promoter comprises the core IL-2 promoter. In general, the naturally occurring IL-2 promoter is relative compact and includes a core promoter containing a TATA box and an upstream regulatory region. The core promoter is considered the region within approximately −40 and +40 nucleotides (e.g., 40 nucleotides upstream (5′) to 40 nucleotides downstream (3′)) of the transcription start site. See, e.g., Weaver et al. Mol. Immunol. (2007) 44(11) 2813-2819.

As used herein, the term “minimal IL-2 promoter” refers to the minimal portion of the IL-2 promoter requires for transcription. In some embodiments, the minimal IL-2 promoter is the IL-2 core promoter. In some embodiments, the NFAT-responsive promoter comprises the core IL-2 promoter comprising a TATA box. A TATA box (also referred to as a “Goldberg-Hogness box”) is a T/A rich sequence found upstream of a transcriptional start site (Shi & Zhou, BMC Bioinformatics (2006) 7, Article number S2). In some embodiments, the TATA box comprises the consensus sequence 5′-TATA(A/T)A(A/T)-3′. The TATA box is thought to be involved in formation of the preinitiation complex for gene transcription and bind a TATA-binding protein (TBP).

In some embodiments, the minimal IL-2 promoter comprises the nucleotide sequence of SEQ ID NO: 95.

An example of a minimal IL-2 promoter is provided by the nucleotide sequence provided by SEQ ID NO: 95:

(SEQ ID NO: 95)
5′-TAGAGGGTATATAATGGAAGCTCGAATTCCA-3′.

In some embodiments, the NFAT binding sites are located 5′ (upstream) of the minimal IL-2 promoter. In some embodiments, the NFAT binding sites are located at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, 50, or more nucleotides 5′ (upstream) of the minimal IL-2 promoter. In some embodiments, the NFAT responsive promoter comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, 50, or more nucleotides between the last NFAT binding site and the minimal IL-2 promoter.

An exemplary nucleotide sequence of a minimal NFAT-responsive promoter is provided by SEQ ID NO: 96. In some embodiments, the nucleotide sequence of the minimal NFAT-responsive promoter comprises, consists of, or consists essentially of the nucleotide sequence of SEQ ID NO: 96, or a sequence that is at least 70%, at least 75%, at least 80%, at least 85%, at least 90%, at least 95%, or at least 99% identical the nucleotide sequence of SEQ ID NO: 96.

Exemplary nucleotide sequence of a minimal NFAT-responsive promoter comprising 6 NFAT binding sites (SEQ ID NO: 96):

(SEQ ID NO: 96)
5′-GGAGGAAAAACTGTTTCATACAGAAGGCGTGGAGGAAAAACTGTTTC
ATACAGAAGGCGTGGAGGAAAAACTGTTTCATACAGAAGGCGTGGAGGAA
AAACTGTTTCATACAGAAGGCGTGGAGGAAAAACTGTTTCATACAGAAGG
CGTGGAGGAAAAACTGTTTCATACAGAAGGCGTGATCTAGACTTAGAGGG
TATATAATGGAAGCTCGAATTCCA-3′

Any of the nucleic acid constructs encoding an IL-2 reporter system described herein may further comprise a nucleotide sequence encoding a second reporter molecule operably linked (under the control of) a constitutive promoter (also referred to as a constitutively active promoter). Preferably, the reporter molecule that is operably linked to the minimal NFAT-responsive promoter is different than the second reporter molecule operably linked to the constitutively active promoter, such that detection of the reporter molecule that is operably linked to the minimal NFAT-responsive promoter is indicative of activity of the NFAT-responsive promoter and detection of the reporter molecule that is operably linked to the constitutively active promoter is indicative of activity of the constitutively active promoter.

In some embodiments, the constitutive promoter controlling expression of the second reporter molecule is referred to as a “reference promoter.” Examples of constitutively active promoter include, without limitation, EF-1alpha (EF1a), CMV promoter, SV40 promoter, PGK1 promoter, Ubc promoter, beta actin promoter, CAG promoter, TRE promoter, UAS promoter, Ac5 promoter, polyhedrin promoter, and U6 promoter. In some embodiments, the constitutively active promoter is an EF1a promoter.

The nucleotide sequence of an elongation factor 1 alpha (EF-1alpha) promoter is provided by the nucleotide sequence of SEQ ID NO: 97.

EF1alpha promoter
(SEQ ID NO: 97)
GGCTCCGGTGCCCGTCAGTGGGCAGAGCGCACATCGCCCACAGTCCCCG
AGAAGTTGGGGGGAGGGGTCGGCAATTGATCCGGTGCCTAGAGAAGGTG
GCGCGGGGTAAACTGGGAAAGTGATGTCGTGTACTGGCTCCGCCTTTTT
CCCGAGGGTGGGGGAGAACCGTATATAAGTGCAGTAGTCGCCGTGAACG
TTCTTTTTCGCAACGGGTTTGCCGCCAGAACACAGGTAAGTGCCGTGTG
TGGTTCCCGCGGGCCTGGCCTCTTTACGGGTTATGGCCCTTGCGTGCCT
TGAATTACTTCCACCTGGCTGCAGTACGTGATTCTTGATCCCGAGCTTC
GGGTTGGAAGTGGGTGGGAGAGTTCGAGGCCTTGCGCTTAAGGAGCCCC
TTCGCCTCGTGCTTGAGTTGAGGCCTGGCCTGGGCGCTGGGGCCGCCGC
GTGCGAATCTGGTGGCACCTTCGCGCCTGTCTCGCTGCTTTCGATAAGT
CTCTAGCCATTTAAAATTTTTGATGACCTGCTGCGACGCTTTTTTTCTG
GCAAGATAGTCTTGTAAATGCGGGCCAAGATCTGCACACTGGTATTTCG
GTTTTTGGGGCCGCGGGCGGCGACGGGGCCCGTGCGTCCCAGCGCACAT
GTTCGGCGAGGCGGGGCCTGCGAGCGCGGCCACCGAGAATCGGACGGGG
GTAGTCTCAAGCTGGCCGGCCTGCTCTGGTGCCTGGCCTCGCGCCGCCG
TGTATCGCCCCGCCCTGGGCGGCAAGGCTGGCCCGGTCGGCACCAGTTG
CGTGAGCGGAAAGATGGCCGCTTCCCGGCCCTGCTGCAGGGAGCTCAAA
ATGGAGGACGCGGCGCTCGGGAGAGCGGGCGGGTGAGTCACCCACACAA
AGGAAAAGGGCCTTTCCGTCCTCAGCCGTCGCTTCATGTGACTCCACGG
AGTACCGGGCGCCGTCCAGGCACCTCGATTAGTTCTCGAGCTTTTGGAG
TACGTCGTCTTTAGGTTGGGGGGAGGGGTTTTATGCGATGGAGTTTCCC
CACACTGAGTGGGTGGAGACTGAAGTTAGGCCAGCTTGGCACTTGATGT
AATTCTCCTTGGAATTTGCCCTTTTTGAGTTTGGATCTTGGTTCATTCT
CAAGCCTCAGACAGTGGTTCAAAGTTTTTTTCTTCCATTTCAGGTGTCG
TGA

The nucleic acid constructs described herein comprise a reporter molecule operably linked (under control of) a minimal NFAT-responsive promoter. In some embodiments, the nucleic acid construct comprises a second reporter molecule operably linked (under control of) a constitutively active promoter. Any suitable reporter molecule(s) may be used in the nucleic acid constructs described herein. Preferably a reporter molecule (a reporter protein) is readily detectable (directly or indirectly) upon expression. In some embodiments, the reporter molecule may be referred to as a screenable marker. Examples of reporter molecules include, without limitation, enzymes, such as β-glucuronidase, α-galactosidase, β-lactamase, and tyrosinase; luciferase; fluorescent markers/proteins. Fluorescent proteins include, but are not limited to, green fluorescent protein (GFP), red fluorescent protein (RFP), blue fluorescent protein (BFP), EBFP, cyan fluorescent protein, ECFP, EG fluorescent protein, yellow fluorescent protein, mWasabi, ZsGreen, yellow fluorescent protein (YFP), ZsYellow, mHoneydew, mApple, mRuby, mBanana, mOrange, mCherry, mCerulean, mTurquoise, mTangerine, mStrawberry, mGrape, mRaspberry, and mPlum. Selection of a suitable reporter molecule, such as a fluorescent protein, may depend on factors such as the means for detecting and/or quantifying the reporter molecule.

The nucleic acid constructs described herein comprise a reporter molecule operably linked (under control of) a minimal NFAT-responsive promoter. In some embodiments, the nucleic acid construct comprises a second reporter molecule operably linked (under control of) a constitutively active promoter. Any suitable reporter molecule(s) may be used in the nucleic acid constructs described herein. Preferably a reporter molecule (a reporter protein) is readily detectable (directly or indirectly) upon expression. In some embodiments, the reporter molecule may be referred to as a screenable marker. Examples of reporter molecules include, without limitation, enzymes, such as β-glucuronidase, α-galactosidase, β-lactamase, and tyrosinase; luciferase; fluorescent markers/proteins. Fluorescent proteins include, but are not limited to, green fluorescent protein (GFP), red fluorescent protein (RFP), blue fluorescent protein (BFP), EBFP, cyan fluorescent protein, ECFP, EG fluorescent protein, yellow fluorescent protein, mWasabi, ZsGreen, yellow fluorescent protein (YFP), ZsYellow, mHoneydew, mApple, mRuby, mBanana, mOrange, mCherry, mCerulean, mTurquoise, mTangerine, mStrawberry, mGrape, mRaspberry, and mPlum. Selection of a suitable reporter molecule, such as a fluorescent protein, may depend on factors such as the means for detecting and/or quantifying the reporter molecule.

In some embodiments, the reporter molecule is a fluorescent protein. In some embodiments, the reporter molecule operably linked to the NFAT-responsive promoter is a fluorescent protein. In some embodiments, fluorescent protein is mTurquoise or mOrange.

A nucleotide sequence encoding mTurquoise is provided by SEQ ID NO: 98.

mTurquoise
(SEQ ID NO: 98)
ATGGTGAGCAAGGGCGAGGAGCTGTTCACCGGGGTGGTGCCCATCCTGG
TCGAGCTGGACGGCGACGTAAACGGCCACAAGTTCAGCGTGTCCGGCGA
GGGCGAGGGCGATGCCACCTACGGCAAGCTGACCCTGAAGTTCATCTGC
ACCACCGGCAAGCTGCCCGTGCCCTGGCCCACCCTCGTGACCACCCTGT
CCTGGGGCGTGCAGTGCTTCGCCCGCTACCCCGACCACATGAAGCAGCA
CGACTTCTTCAAGTCCGCCATGCCCGAAGGCTACGTCCAGGAGCGCACC
ATCTTCTTCAAGGACGACGGCAACTACAAGACCCGCGCCGAGGTGAAGT
TCGAGGGCGACACCCTGGTGAACCGCATCGAGCTGAAGGGCATCGACTT
CAAGGAGGACGGCAACATCCTGGGGCACAAGCTGGAGTACAACTACTTT
AGCGACAACGTCTATATCACCGCCGACAAGCAGAAGAACGGCATCAAGG
CCAACTTCAAGATCCGCCACAACATCGAGGACGGCGGCGTGCAGCTCGC
CGACCACTACCAGCAGAACACCCCCATCGGCGACGGCCCCGTGCTGCTG
CCCGACAACCACTACCTGAGCACCCAGTCCAAGCTGAGCAAAGACCCCA
ACGAGAAGCGCGATCACATGGTCCTGCTGGAGTTCGTGACCGCCGCCGG
GATCACTCTCGGCATGGACGAGCTGTACAAG

A nucleotide sequence encoding mOrange is provided by SEQ ID NO: 99.

mOrange
(SEQ ID NO: 99)
ATGGTGAGCAAGGGCGAGGAGAATAACATGGCCATCATCAAGGAGTTCA
TGCGCTTCAAGGTGCGCATGGAGGGCTCCGTGAACGGCCACGAGTTCGA
GATCGAGGGCGAGGGCGAGGGCCGCCCCTACGAGGGCTTTCAGACCGCT
AAGCTGAAGGTGACCAAGGGTGGCCCCCTGCCCTTCGCCTGGGACATCC
TGTCCCCTCATTTCACCTACGGCTCCAAGGCCTACGTGAAGCACCCCGC
CGACATCCCCGACTACTTCAAGCTGTCCTTCCCCGAGGGCTTCAAGTGG
GAGCGCGTGATGAACTACGAGGACGGCGGCGTGGTGACCGTGACCCAGG
ACTCCTCCCTGCAGGACGGCGAGTTCATCTACAAGGTGAAGCTGCGCGG
CACCAACTTCCCCTCCGACGGCCCCGTGATGCAGAAGAAGACCATGGGC
TGGGAGGCCTCCTCCGAGCGGATGTACCCCGAGGACGGTGCCCTGAAGG
GCAAGATCAAGATGAGGCTGAAGCTGAAGGACGGCGACATCAAGTTGGA
CATCACCTCCCACAACGAGGACTACACCATCGTGGAACAGTACGAACGC
GCCGAGGGCCGCCACTCCACCGGCGGCATGGACGAGCTGTACAAG

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.): Handbook of Experimental Immunology (D. M. Weir and C. C. Blackwell, eds.): Gene Transfer Vectors for Mammalian Cells (J. M. Miller and M. P. Calos, eds., 1987; Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They are not to be construed as limiting the scope or content of the disclosure in any way.

### EXAMPLES

### Example 1. Generation of Novel Antibodies Against CD33

A naïve llama phage library was panned and screened to identify single domain antibodies that specifically recognize CD33. A MOLM-13 cell line expressing CD33 was used as a CD33-positive cell line, and a MOLM-13 cell line harboring a knockout of CD33 was used as a CD-33 negative cell line. A purified anti-human CD33 antibody was used as a positive control. Specific binding activity of identified binders was confirmed by FACS. The heavy chain variable region sequences of the identified binders are included herein.

### Example 2. Generation and Evaluation of CAR Constructs

CAR Constructs

CAR constructs are developed with CD33-specific single domain antibody fragments (sdAb) described herein. A CAR construct comprises a sdAb linked with either a CD8a or CD28 transmembrane domain, paired with either a 4-1BB or CD28 co-stimulatory domain, and a CD3ζ (zeta) signaling domain. The CAR sequences are cloned in a third-generation lentiviral plasmid.

Cell Lines

The GFP and luciferase expressing AML cells lines MV411, THP1, and MOLM14 containing varying levels of CD33 expression and different genotypes for an exon 2 splice variance (Laszlo et al., Oncotarget, 7: 43281-94 (2016)) are used to test the efficacy of the CAR constructs described above. MV411 is an acute monocytic leukemia line established from a 10-year-old boy with acute monocytic leukemia (AML FAB M5). THP-1 is a human monocytic cell line derived from an acute monocytic leukemia patient. MOLM14 is an acute myeloid leukemia line established from the peripheral blood of a 20-year-old man with acute myeloid leukemia (AML FAB M5a) at relapse in 1995 after initial myelodysplastic syndrome (MDS, refractory anemia with excess of blasts, RAEB). Through DNA isolation, it was found that MOLM14 has a CC genotype and does not contain the SNP, while TE1P1 and MV411 are both heterozygous for the SNP with the CT genotype (Lamba et al., J. Clin. Oncol., 35: 2674-82 (2017)). This cell line does not express neither CD33 nor CD123. K562 is a human erythroleukemia leukemia line established and derived from a 53-year-old female chronic myelogenous leukemia patient.

CAR T-Cell Generation

CD33 CAR-encoding lentiviral vectors are produced by transient transfection of the Lenti-X 293T lenti packaging cell line Lenti-X 293T cells and plated into poly-D lysine coated 15-cm plates (BD Biosciences, San Jose, CA, USA). The following day, Lenti-X 293T cells are transfected using lipofectamine 3000 (Thermo Fisher Scientific, Waltham, MA, USA) with plasmids encoding the CAR along with packaging and envelope vectors (pMDLg/pRRE, pMD-2G, and pRSV-Rev). Lentiviral supernatants are harvested at 24 and 48 hours post-transfection, centrifuged at 3000 RPM for 10 minutes to remove cell debris, and frozen on dry ice and stored at −80° C. Human PBMCs from normal donors are obtained with an NIH-approved protocol and activated with a 1:3 ratio of CD3/CD28 microbeads (Dynabeads Human T-Expander CD3/CD28, Thermo Fisher Scientific, Cat #11141D) in AIM-V media containing 40 IU/mL recombinant IL-2 and 5% FBS for 24 hours. Activated T cells are re-suspended at 2 million cells per 2 mL of lentiviral supernatant plus 1 mL of fresh AIM-V media with 10 mcg/mL protamine sulfate and 100 IU/mL IL-2 in 6-well plates. Plates are centrifuged at 1000×g for 2 hours at 32° C. and incubated overnight at 37° C. A second transduction is performed on the following day by repeating the same transduction procedure described above. The CD3/CD28 beads are removed on the third day following transduction, and the cells are cultured at 300,000 cells/mL in AIM-V containing 100 IU/mL IL2 with fresh IL2-containing media added every 2-3 days until harvest on day 8 or 9.

Flow Cytometry

Surface expression of CD33 CAR-transduced T cells is determined by flow cytometry using either protein-L (Thermo Fisher) or a Biotinylated Human Siglec-3/CD33 Protein (Aero Biosystems, Newark, DE, USA) followed by incubation with Streptavidin-PE (BioLegend, San Diego, CA, USA).

PDX

1 million cells of a PDX leukemia cell line JMM117 are injected into the NSG mice one week ahead of adoptive CAR T cell transfer. The mice are treated with CAR T cells on day 0. Two weeks later, the mice are taken down and analysis is performed.

Cytotoxicity Assay

5E4 of Target tumor cells in 100 μl of RPMI media are loaded into a 96-well plate (Corning® (Croning, NY) BioCoat™ Poly-L-Lysine 96-Well Clear TC-Treated Flat Bottom Assay Plate). An equal amount of CAR T cells are added into the designated well on the following day. The initial incucyte apoptosis marker (Essen BioScience, Ann Arbor, MI, USA) is diluted in 100 μL PBS and 1 μl of the diluent is added into each well. The plate is scanned for the GFP and or RFP fluorescent expression to monitor the cell apoptosis using an IncuCyte ZOOM® system every 30 minutes in a duration of 40 hours. The percentage of cell killing at each time point is baseline-corrected.

Analysis of Cytokine Production

Target tumor cell and transduced CAR-positive T cells are washed 3 times with 1×PBS and re-suspended in RPMI at 1E6/mL. 100 μL of tumor cells with 100 μL of CAR positive T cells are loaded into each well of a 96-well plate. T cell only and tumor cell only controls are set up. All tests are performed in duplicate or triplicate. Cells are incubated for 18 hours at 37° C. and 120 μL of the culture supernatant was harvested for detection of cytokine production. Cytokine levels in supernatants are measured using either ELISA kits (R&D Systems, Minneapolis, MN, EISA) or a multiplex assay (Meso Scale Discovery, Rockville, MD, EISA).

Bioenergetic Analyses

For a glycolysis stress test, CAR-T cells are suspended in serum-free unbuffered DMEM medium (Sigma-Aldrich, St. Louis, MO, USA) supplemented with L-glutamine (200 mM) and NaCl (143 mM). 0.6 mL of a 0.5% Phenol Red solution (SigmaP0290) is added for a final concentration of 3 mg/L and to adjust the pH to 7.35+/−0.05. CAR-T cells are plated onto Seahorse cell plates (3E5 cells per well), coated with Cell-Tak (Corning) to facilitate T cell attachment. Briefly, the cartridges are hydrated the day before the assay. On the day of the assay, the plates are coated with Cell-Tak and the cells are seeded in the Cell-Tak coated plates and placed on the XF24 Analyzer for the assay. The detailed procedure is as follows.

The assay cartridge is initially hydrated with XF calibrant solution at 200 μL/well, hydro booster is added, and is wrapped in parafilm, and the sensor cartridge is placed on top of utility plate and incubated at 37° C. without CO₂ for overnight. The cell culture plate is then coated with Cell-Tak as follows: For 1 plate, 46 mL of Cell-Tak was diluted in 204 mL TC water and 1 mL of NaHCO₃. The mixer is dispensed 50 mL in each well and the plate is incubated at room temperature for at least 20 minutes. After removing the Cell-Tak solution, 250 mL of TC water is used to wash each well. CAR-T cells (3E5/well) are plated in 158 mL assay media. The cell culture plate is then spun at 450 rpm for 1 second at slow acceleration and no deceleration, and then the plate is reversed in orientation and spun at 650 rpm for 1 second at slow acceleration and no deceleration. The plate is then incubated at 37° C. 0% C0₂ for 25-30 minutes.

After 25-30 minutes incubation, 158 μL of warm assay medium is added slowly and gently to the top of each well along the side of the wall using a manual P200 pipettor. The cell plates are incubated for 15-25 minutes. After 15-25 minutes, the plates are placed on XF24 Analyzer (after calibration finished). An XF assay is then executed. Solution is injected sequentially through three ports: Port A: glucose 80 mM (96 mL of the stock solution in 3 mL assay media). Port B: oligomycin 18 mM (10.8 mL of the stock solution in 3 mL assay media). Port C: 2DG use stock solution. A glycolysis stress test is performed by measuring ECAR (mpH/min) at steady state after the cartridge ports are loaded with 75 mL of drug solution.

For a mitochondrial stress test, CAR T cells are suspended in serum-free unbuffered DMEM medium with D-glucose (25 mM), and sodium pyruvate (1 mM). Mitochondrial stress test is performed similarly as the above by measuring OCR (pmol/min) at steady state and after sequential injection of oligomycin (0.5 mM), FCCP (0.5 mM), rotenone (1 mM) and antimycin A (1 mM) (Sigma-Aldrich). Experiments with the Seahorse system utilize the following assay conditions: 2 minutes mixture; 2 minutes wait; and 3 minutes measurement. All samples are tested in six replicates.

Fluorescence Microscopy Imaging and Analysis

MOLM14 (4×10s) tumor cells are plated in 1 mL of warm RPMI on the Cell-tak coated inner well of an ibidi m-Dish 35 mm and incubated overnight in a 37° C. incubator. Tumor cells are then stained with Hoechst Dye (2.5 μg/mL). T cells are transduced to express CAR-mCherry fusion proteins. CAR-T positive cells are sorted and then 7.5 E5 of these CAR-T cells are incubated with the fixed MOLM14 cell in the dish for an hour. The cells are subsequently washed and fixed with freshly prepared 4% paraformaldehyde and mounted in a non-hardening mounting media in preparation for imaging.

To evaluate actin expression at the immune synapse, the above protocol is modified, and samples are permeabilized with 0.1% triton x after paraformaldehyde fixation. Cells are stained with Phalloidin 640 (165 nM) and then washed prior to mounting. Airyscan images are acquired using a Zeiss LSM 880. The exposure setting is the same for the entire experiment. Images are collected as a z stack to cover the entire volume of the immune synapse.

Images are acquired using a Nikon Eclipse Ti2 spinning disc confocal microscope with 63× objective. Z stacks of 0.5 μM thickness are acquired in parallel over a range of 10 μM above and below the focal plane for the three channels (405, 488, 640 nm). Each channel is excited at 50% laser intensity with exposure times of 300 ms, 1 s, and 300 ms for 405, 488, and 640, respectively. ImageJ software is used for data analysis.

Quantitative analysis for n>10 immune synapses for each CAR is performed to evaluate CAR and actin accumulation. Specifically, the ratio of mean fluorescence intensity (MFI) at the synapse vs. ratio of the MFI at the rest of the T cell surface is determined. Additional parameters include ratio of MFPvolume at the IS vs. MFI* volume for the rest of the T cell surface, MF volume of IS vs. MFI*volume of T cell, and intracellular CAR signal vs. extracellular CAR signal is also evaluated. For actin, fluorescence intensity at the IS are normalized against the baseline actin T cell expression. MFPvolume of actin at the IS are determined and MFI* volume of unengaged T and tumor cells are subtracted to account for baseline actin expression.

In Vivo Experiments

Animal experiments are carried out under protocols approved by an Animal Care and Use Committee. AML cell lines and the xenografted human AML specimens are IV injected into NSG mice. For luciferase-expressing lines, leukemia is detected using the Xenogen IVIS Lumina (Caliper Life Sciences, Hopkinton, MA, USA). NSG are injected intraperitoneally with 3 mg D-luciferin (Caliper Life Sciences) and are imaged 4 minutes later with an exposure time of 1 min for AML cell lines. Living Image Version 4.1 software (Caliper Life Sciences) is used to analyze the total bioluminescent signal flux for each mouse as photons. At time of take down, bone marrow, spleen, and liver of mice are harvested assessed by flow cytometry.

Statistical Analysis

Statistics analysis is performed using Prism 7.0 software. Plots are presented as mean+/−SD. Statistical significance of all data is calculated using an unpaired student t test. p<0.05 is considered as significant.

### Example 3: Epitope Mapping and Evaluation of CAR Constructs

Understanding the CD33 epitopes to which anti-CD33 CAR constructs bind may enable improvements in anti-CD33 CAR construct design and therapeutic application, e.g., enabling use of combinations of anti-CD33 binding domains that target CD33 and do not interfere with one another. An Octet® Red 96 assay was performed to evaluate anti-CD33 single domain antibody fragments (sdAbs) and determine whether a given two anti-CD33 sdAbs compete for the same binding sites on the CD33 molecule.

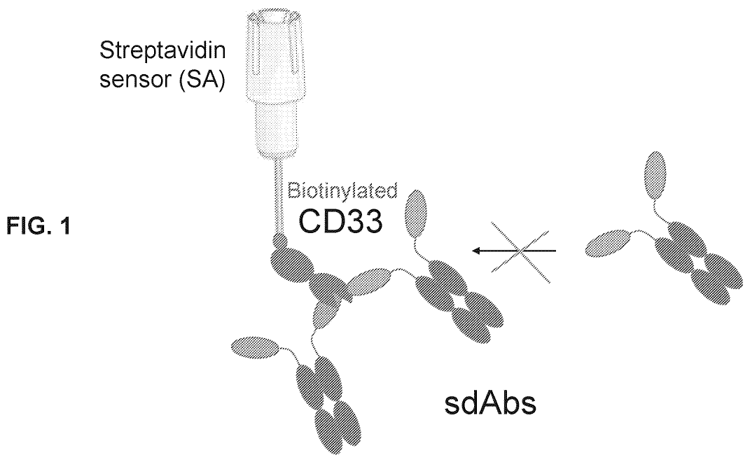

Biotinylated CD33 protein is captured on a streptavidin (SA) sensor and the first sdAbs is bound to CD33 (FIG. 1). Then the second antibody is checked for binding. Binding of the first sdAb to CD33 induces a change in the light signal and the change in absorption is reflected as a curve and the addition of the second antibody may or may not induce a similar curve. A lack of increase in signal when the second antibody is added suggests that the second antibody competes for the same binding site as the first antibody. An increase in signal when the second antibody is added, suggests that there is no competition between the two antibodies.

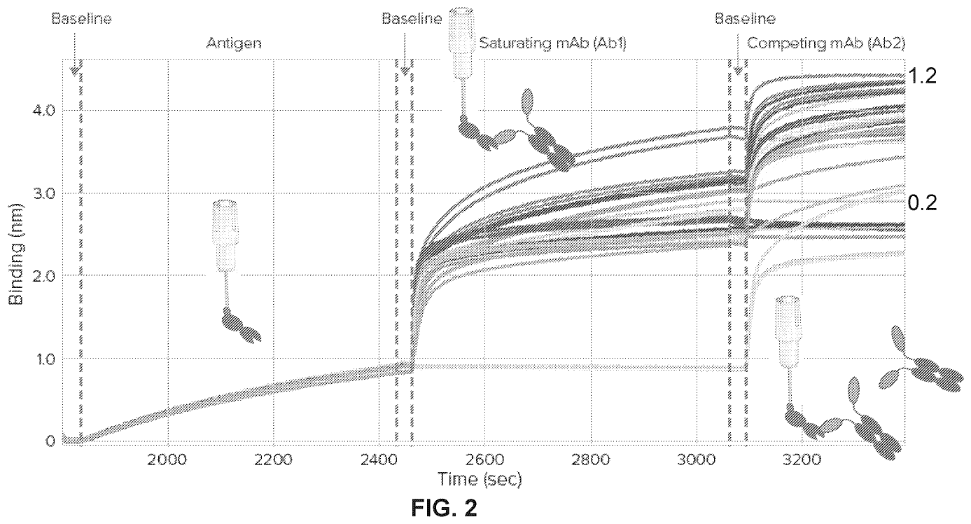

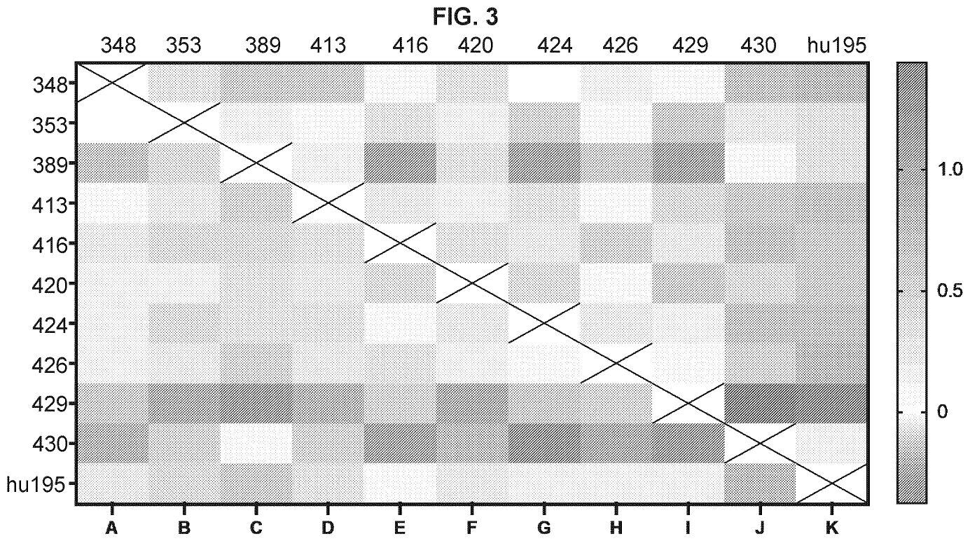

Ten exemplary anti-CD33 sdAbs of the disclosure were tested using the Octet biotinylated-CD33 SA sensor in competitive binding against one another (FIGS. 2 and 3) and a control anti-CD33 antibody, hu195. In the heatmap representation of the data, signal for second antibody binding (and thus a lack of competition) is shown by green and lack of signal for second antibody binding (and thus competition in binding between first and second antibodies) is shown by yellow and red. Data from FIG. 3 is provided in Table 1.

TABLE 1
Numerical Data from FIG. 3

| AB# | 348 | 353 | 389 | 413 | 416 | 420 | 424 | 426 | 429 | 430 | h195 |
|---|---|---|---|---|---|---|---|---|---|---|---|
| 348 | — | 0.3219 | 0.5838 | 0.6233 | 0.0635 | 0.3805 | 0.0143 | 0.1583 | 0.0422 | 0.6512 | 0.7988 |
| 353 | 0.0077 | — | 0.1518 | 0.0204 | −0.0696 | −0.0387 | −0.1077 | −0.0122 | −0.1296 | 0.2314 | 0.2952 |
| 389 | −0.1456 | −0.1005 | — | −0.0267 | −0.2797 | −0.0649 | −0.2709 | −0.1369 | −0.2601 | 0.0905 | 0.3548 |
| 413 | −0.024 | 0.2477 | 0.4832 | — | −0.0586 | 0.1497 | −0.0796 | 0.0667 | −0.0887 | 0.6296 | 0.6478 |
| 416 | 0.2287 | 0.4379 | 0.4564 | 0.3756 | — | 0.3396 | 0.2198 | 0.5139 | 0.2608 | 0.6968 | 0.565 |
| 420 | 0.1387 | 0.1833 | 0.376 | 0.2 | −0.094 | — | −0.0931 | 0.0276 | −0.124 | 0.4565 | 0.5786 |
| 424 | 0.1542 | 0.4437 | 0.3562 | 0.3703 | 0.086 | 0.266 | — | 0.239 | 0.1627 | 0.7327 | 0.6483 |
| 426 | 0.1833 | 0.2589 | 0.5304 | 0.2572 | 0.3042 | 0.1906 | 0.0441 | — | 0.0225 | 0.5381 | 0.7433 |
| 429 | 0.616 | 1.0142 | 1.1629 | 0.8645 | 0.5524 | 0.9497 | 0.6276 | 0.5435 | — | 1.4405 | 1.3678 |
| 430 | −0.2119 | −0.1116 | 0.0436 | −0.1084 | −0.2683 | −0.1694 | −0.3765 | −0.2267 | −0.2904 | — | 0.1358 |
| h195 | 0.2634 | 0.3987 | 0.5851 | 0.3223 | 0.0872 | 0.2632 | 0.1624 | 0.1257 | 0.1467 | 0.8139 | — |

| | |
|---|---|
| Scale | |
| −0.4 to 0 | Competition |
| 0.1 to 0.6 | Moderate competition |
| 0.61 to 1.5 | No competition |

The results show that the sdAbs tested may be divided into competition groups that bind to the same or overlapping epitopes of CD33: Group 1 (sdAbs 389 and 430), Group 2 (sdAbs 353, 413, and 420), and Group 3 (sdAbs 348, 416, 424, and 426).

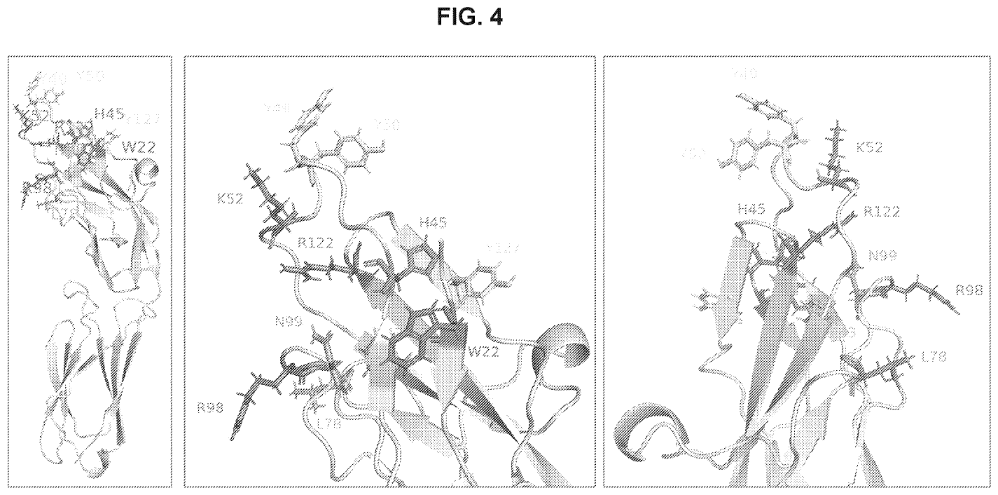

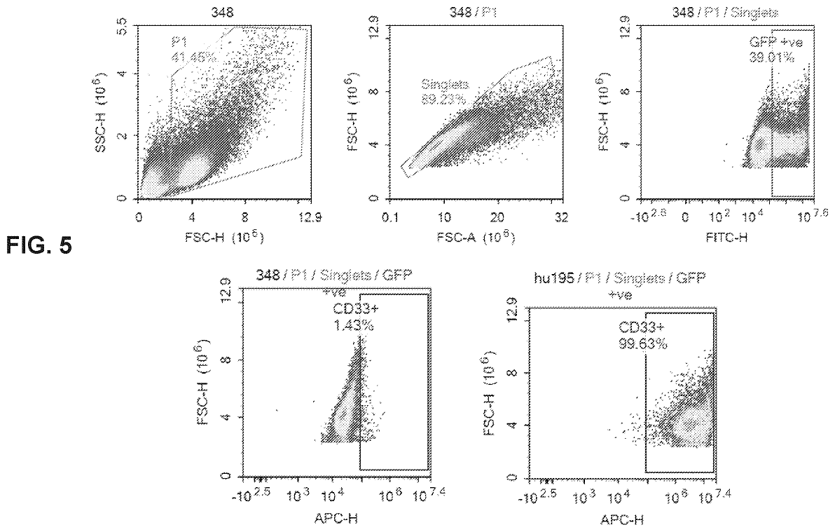

In silico modeling experiments docking CD33 and model antibodies were used to predict amino acid residues on CD33 in the vicinity of anti-CD33 sdAb CDRs. CD33 amino acids within 2.5-3.0 Å from CDRs were selected for mutagenesis. Residues were mutated to alanines or were changed to an opposite charge or changed to either polar to hydrophobic residues or vice versa (FIG. 4). Mutant CD33-GFP constructs were transiently transfected into 293FT cells, and approximately 48 hours post transfection the 293FT cells were incubated with anti-CD33 sdAbs. FACS was used to detect sdAb binding to the mutant CD33-GFP constructs. See example FACS data in FIG. 5, showing cell gating of live W22A CD33 293FT cells (P1, top left), singlet non-double cells (top middle), GFP positive cells (top right), and finally CD33 mutant binding for sdAb 348 (bottom left) and hu195 (bottom right). Table 2 lists the CD33 mutants tested and the data for the 10 sdAbs tested; hu195 was used as a control. ‘AEAD’ was a quadruple mutant CD33 to which no sdAb bound or which did not express on the 293FT.

TABLE 2
sdAb FACS Binding Data Part 1

| | M1 | M2 | M3 | M4 | M5 |
|---|---|---|---|---|---|
| | (W22A) | (H45E) | (N99A) | (R122D) | (AEAD) |
| hu195 | 99.33 | 99.63 | 99.52 | 99.63 | 0.26 |
| 348 | 8.08 | 2.54 | 81.86 | 1.43 | 0.41 |
| 353 | 99.38 | 98.82 | 99.44 | 98.52 | 0.31 |
| 389 | 99.33 | 84.41 | 96.96 | 6.01 | 0.16 |
| 420 | 98.41 | 91.54 | 98.93 | 97.75 | 0.15 |
| 430 | 99.42 | 99.34 | 99.3 | 98.37 | 0.79 |
| 413 | 76.42 | 5.93 | 96.84 | 75.96 | 0.81 |
| 416 | 1.25 | 96.59 | 98.72 | 99.32 | 0.24 |
| 424 | 80.12 | 97.33 | 3.43 | 98.6 | 0.07 |
| 426 | 99.5 | 97.71 | 99.48 | 95.9 | 1.2 |
| 429 | 99.12 | 65.5 | 98.98 | 21.14 | 0.12 |

TABLE 2
sdAb FACS Binding Data Part 2

| | M6 | M7 | M8 | M9 | M10 |
|---|---|---|---|---|---|
| | (F43N) | (R98D) | (K52A) | (Y49, 50A) | (Y127A) |
| hu195 | 99.44 | 98.43 | 99.52 | 94.37 | 92.45 |
| 348 | 95.31 | 0.48 | 91.24 | 0.39 | 98.45 |
| 353 | 98.97 | 98.78 | 97.31 | 0.64 | 96.27 |
| 389 | 98.26 | 21.5 | 0.15 | 97.36 | 97.21 |
| 420 | 98.29 | 74.79 | 8.64 | 34.63 | 98.11 |
| 430 | 98.8 | 98.52 | 97.07 | 1.01 | 94.21 |
| 413 | 97.54 | 97.05 | 91.37 | 0.79 | 90.18 |
| 416 | 97.87 | 98.6 | 98.91 | 99.12 | 98.91 |
| 424 | 98.57 | 97.16 | 95.36 | 0.23 | 83.71 |
| 426 | 99.15 | 99.11 | 96.79 | 1.25 | 96.09 |
| 429 | 97.58 | 97.09 | 66.08 | 0.22 | 93.93 |

TABLE 2
sdAb FACS Binding Data Part 3

| | M11 | M12 | M13 | M14 | M15 |
|---|---|---|---|---|---|
| | (L78T) | (G88S) | (R91E) | (E170R) | (Q171A) |
| hu195 | 86.75 | 98.29 | 98.09 | 98.2 | 97.56 |
| 348 | 33.37 | 98.22 | 98.57 | 98.23 | 98.68 |
| 353 | 77.99 | 99.01 | 98.68 | 98.66 | 98.21 |
| 389 | 62.83 | 99.01 | 99.07 | 98.85 | 98.79 |
| 420 | 72.39 | 98.74 | 98.77 | 98.74 | 98.91 |
| 430 | 73.75 | 98.49 | 98.7 | 97.42 | 97.21 |
| 413 | 58.51 | 98.57 | 98.51 | 98.24 | 98.61 |
| 416 | 45.12 | 99.08 | 99.25 | 98.96 | 98.89 |
| 424 | 65.94 | 98.77 | 99.07 | 98.77 | 98.73 |
| 426 | 77.83 | 99.18 | 99.34 | 99.15 | 99.28 |
| 429 | 67.35 | 98.63 | 98.51 | 98.43 | 98.94 |

TABLE 2
sdAb FACS Binding Data Part 4

| | M16 | M17 | M18 | M19 | |
|---|---|---|---|---|---|
| | (4889AII) | (F21Y) | (353AAL) | (348ATAI) | N20D |
| hu195 | 97.19 | 98.72 | 99.41 | 99.69 | 1.31 |
| 348 | 98.37 | 98.88 | 97.34 | 98.96 | 98.93 |
| 353 | 98.36 | 98.17 | 99.02 | 99.62 | 98.69 |
| 389 | 98.73 | 98.54 | 99.45 | 99.59 | 98.46 |
| 420 | 98.55 | 99.11 | 99.09 | 99.41 | 99.07 |
| 430 | 97.4 | 96.06 | 99.39 | 99.75 | 97.54 |
| 413 | 98.3 | 84.62 | 49.24 | 99.35 | 56.68 |
| 416 | 80.78 | 99.13 | 99.82 | 99.57 | 98.99 |
| 424 | 98.48 | 99.03 | 70.24 | 99.53 | 98.31 |
| 426 | 99.03 | 99.12 | 99.29 | 99.75 | 99.11 |
| 429 | 98.58 | 94.1 | 15.56 | 98.31 | 96.59 |

TABLE 2
sdAb FACS Binding Data Part 5

| | JS23 | JS24 | JS25 | JS28 | |
|---|---|---|---|---|---|
| | (L23N) | (Q24E) | (V25M) | (S28L) | WT |
| hu195 | 99.39 | 72.49 | 99.79 | 99.88 | 97.76 |
| 348 | 95.41 | 98.76 | 99.34 | 99.51 | 98.27 |
| 353 | 99.06 | 99.44 | 99.7 | 99.62 | 98.34 |
| 389 | 99.04 | 99.31 | 99.54 | 99.6 | 98.41 |
| 420 | 98.7 | 99.06 | 99.45 | 99.57 | 98.58 |
| 430 | 99.19 | 99.56 | 99.68 | 99.7 | 97.56 |
| 413 | 98.42 | 97.02 | 99.48 | 99.43 | 98.04 |
| 416 | 94.31 | 98.62 | 99.03 | 99.63 | 98.77 |
| 424 | 97.8 | 99.38 | 99.61 | 99.59 | 98.64 |
| 426 | 99.2 | 99.38 | 99.74 | 99.73 | 99.17 |
| 429 | 98.43 | 98.22 | 99.58 | 99.6 | 98.02 |

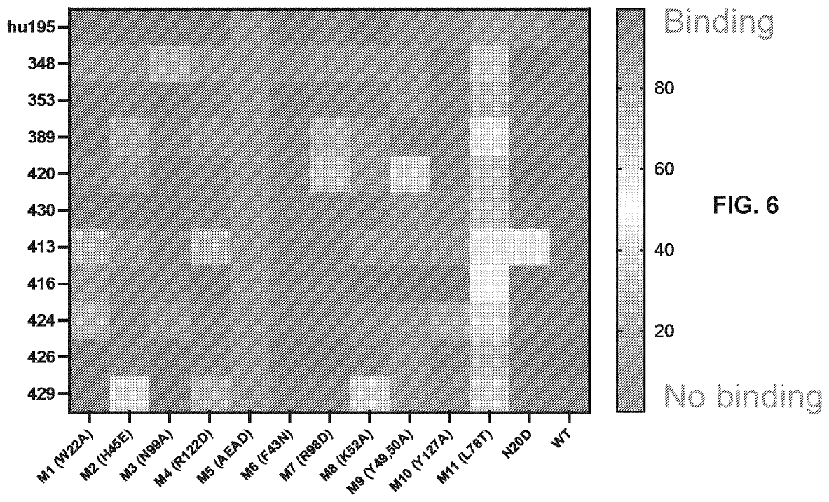

FIG. 6 shows a selection of sdAb binding data from Table 2 in the form of a heatmap. If an sdAb lost binding to a CD33 point mutant, then that residue was determined to be important/critical for binding. In some embodiments, multiple residues form an epitope and thus more than one residue may be critical for sdAb binding.

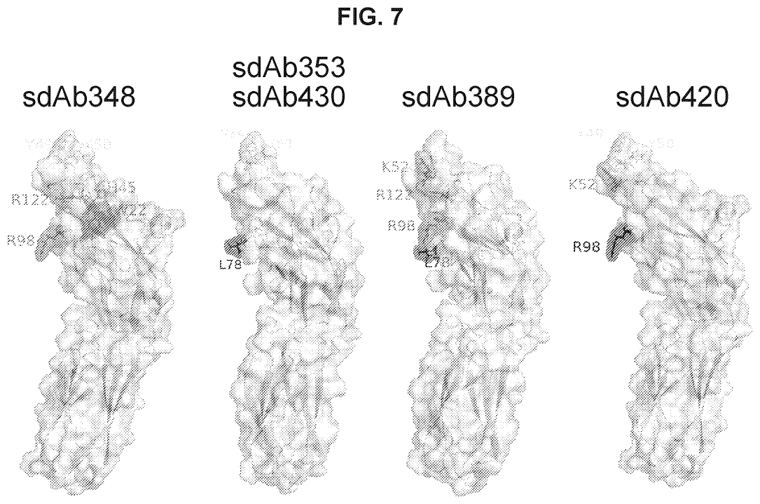

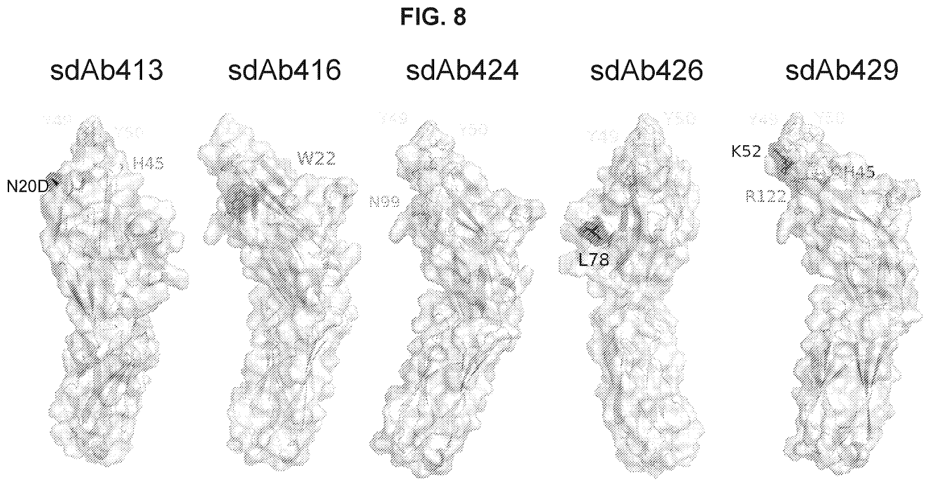

FIGS. 7 and 8 show structural models of CD33 highlighting amino acids determined to be important for binding of the indicated anti-CD33 sdAb. Table 3 summarizes the amino acid site data.

TABLE 3
sdAb CD33 Binding Site Summary

| Antibody | Amino acids |
|---|---|
| 348 | W22, H45, Y49, Y50, R98, R122 |
| 353 | Y49, Y50, L78 |
| 389 | K52, L78, R98, R122 |
| 420 | Y49, Y50, K52, R98 |
| 413 | N20, H45, Y49, Y50 |
| 416 | W22 |
| 424 | Y49, Y50, N99 |
| 426 | Y49, Y50, L78 |
| 429 | H45, Y49, Y50, K52, R122 |
| 430 | Y59, Y50, L78 |

### Example 4: Establishing a T Cell Activation Reporter System

Exemplary nucleic acid constructs were designed to encode a reporter molecule operably linked to a minimal NFAT-responsive promoter and a second reporter molecule operably linked to a constitutive promoter (e.g., EF1a). The minimal NFAT-responsive promoter contained 6 NFAT binding sites upstream of a minimal IL-2 promoter comprising a TATA box and the coding sequence of the reporter molecule. The nucleic acids were produced using conventional methods known in the art.

The first nucleic acid construct (EF1a_mOrange_IL-2_mTurq) contained the mOrange reporter molecule under control of the constitutively active E1 Falpha promoter and mTurquoise reporter molecule (mTurq) under control of the minimal NFAT-responsive promoter. The second nucleic acid construct (EF1a_mTurq_IL-2_mOrange) contained the mTurquoise reporter molecule under control of the constitutively active E1Falpha promoter and mOrange reporter molecule under control of the minimal NFAT-responsive promoter.

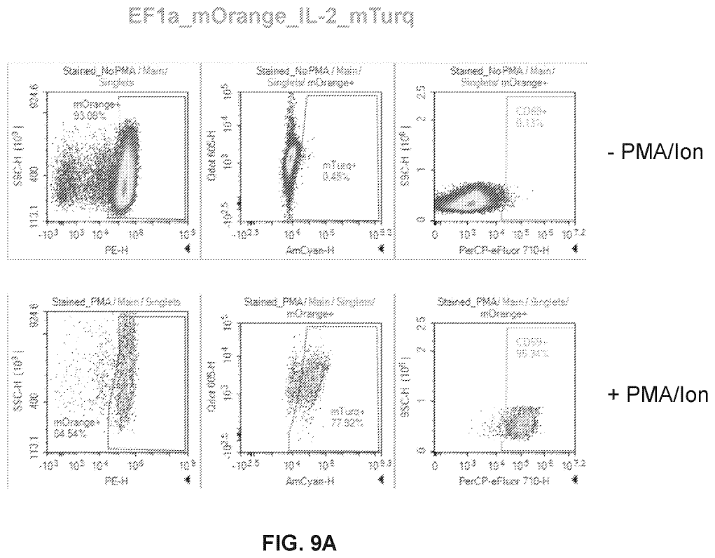

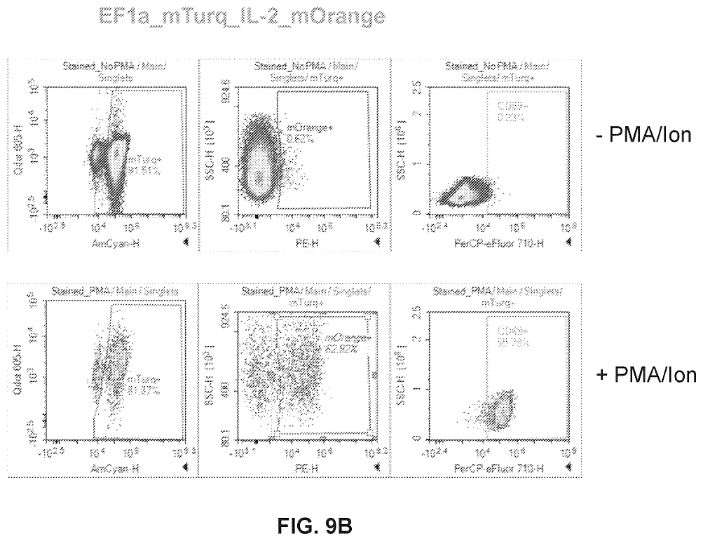

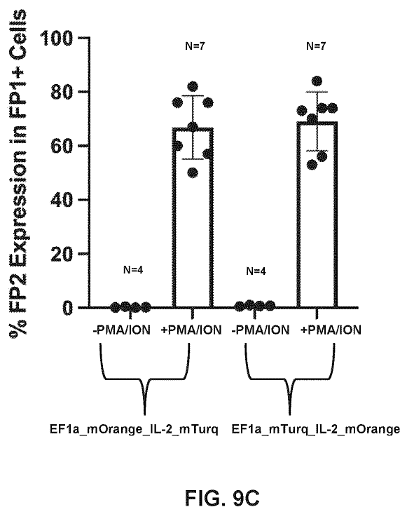

Two IL-2 reporter cell lines were generated by transducing the lentiviral vectors into Jurkat cells. 1×10⁶ cells/mL were activated using 2 μL phorbol myristate acetate (PMA) and ionomycin (a T-cell activation cocktail (see, e.g., BioLegend Activation Cocktail) for 24 hours and assessed for expression of each of the reporter molecules as well as CD69, an indicator of T cell activation, using flow cytometry. As shown in FIGS. 9A and 9B, expression of the reporter molecule under control of the minimal NFAT-responsive promoter was minimally detected when cells were not activated, which significantly increased when cells were activated with PMA/ionomycin. In contrast, expression of the reporter molecule under control of EF1a (the constitutive promoter) was detected in the presence and absence of cell activation. Expression of the reporter molecule under control of the minimal NFAT-responsive promoter was normalized to the expression of the reporter molecule under control of EF1a (the constitutive promoter). See, FIG. 9C.

These results indicate that the minimal NFAT-responsive promoter induces expression of the reporter molecule when activated. Expression of the reporter molecule under control of the minimal NFAT-responsive promoter relative to expression of reporter molecule under control of EF1a (the constitutive promoter) provides a means of normalizing expression to account for factors, such as any differing transduction efficiencies between the constructs.

### Example 5: Evaluating CAR Constructs Using a Reporter System

CAR constructs were designed to target CD33, as described in Example 2. CD33, also known as Siglec (Sialic-acid-binding immunoglobulin-like lectin) plays a role in mediating cell-cell interactions and in maintaining immune cells in a resting state. CD33 is expressed on the surface of the vast majority of AML blasts and chronic myeloid leukemia in blast crisis. It is also aberrantly expressed on a subset of T cell acute lymphoblastic leukemias. Normal tissue expression is restricted to normal myeloid cells. Currently, treating AML with a therapy that targets CD33 can be effective, but the therapy may be limited in utility due to toxicity to the normal blood and bone marrow. The methods described herein allow for comparison of CAR constructs, such as the activity and function of the CAR constructs, as well as high-throughput screening methods for identifying CAR constructs having desired properties (e.g., level of activation of T cells). Example CAR constructs are known in the art. See for example, PCT Publication No. WO 2019/178382 A1, as well as Kenderian, et al. Leukemia (2015) 29: 1637-1647.

Reporter cells containing the exemplary nucleic acid construct EF1a_mOrange_IL-2_mTurq or EF1a_mTurq_IL-2_mOrange were generated as described in Example 2. The cells were transduced with the 8 different CD33 CARs shown in Tables 1 and 5. Cells were co-cultured for 24 hours with either wild-type MOLM-13 cells (CD33+) or MOLM-13 cells that are deficient for CD33 (MOLM-13 CD33KO).

Following co-culture, expression of the reporter molecules was assessed by flow cytometry. Cells were pre-gated on Jurkat cells displaying the fluorescent marker linked to the EF1a promoter, which indicates cells that were transduced with the nucleic acid construct and are able to express the construct. Next, expression of the IL2 linked fluorescent reporter was determined in each co-culture for each of the CD33 CAR constructs as a percentage of constitutive-fluorescence-positive cells (e.g., in cells transduced with EF1a_mOrange_IL-2_mTurq, the expression of mTurq as a percentage of mOrange-positive cells). A ratio was determined for expression of the NFAT-inducible reporter when co-cultured in the presence of wild-type MOLM-13 cells relative to expression of the NFAT-inducible reporter when co-cultured in the presence to MOLM-13 CD33KO cells to determine activity of the CD33 CAR (CD33-specific activation). See, Table 4.

Results indicate that the IL-2 reporter system cells can be used as an objective and reliable reporter system for comparing activity of CAR constructs. Assessing expression of a reporter molecule that is constitutively expressed eliminates false outcomes, potentially due to altered transduction efficiencies, and verifies successful transduction of the reporter construct. Expression of the reporter molecule, driven only in activated cells, represents antigen recognition by and activity of the CAR construct.

TABLE 4
T Cell Activation Results for Table 1 Anti-CD33-CARs

| | Baseline FP2 | Testing FP2 | |
|---|---|---|---|
| | Expression | Expression | |
| CD33CAR | (CD33KO) | (CD33+) | Ratio |
| 1 | NA | NA | NA |
| 2 | 4.67 | 27.79 | 5.95 |
| 3 | 2.7 | 3.9 | 1.44 |
| 4 | 1.72 | 4.14 | 2.41 |
| 5 | 3.32 | 28.21 | 8.50 |
| 6 | 6.36 | 22.89 | 3.60 |
| 7 | 8.87 | 17.55 | 1.98 |
| 8 | 5.01 | 29.36 | 5.86 |

The results show that the anti-CD33 CARs comprising the anti-CD33 antibodies of the disclosure (CD33 CARs 5-8) show T cell activating activity greater than at least one previously known anti-CD33 CAR. Anti-CD33-CAR5 showed the highest T cell activating activity of all anti-CD33 CARs tested. These results demonstrate the potential of anti-CD33 CARs of the present disclosure in the construction of CAR T therapeutics targeting CD33-expressing cancers.

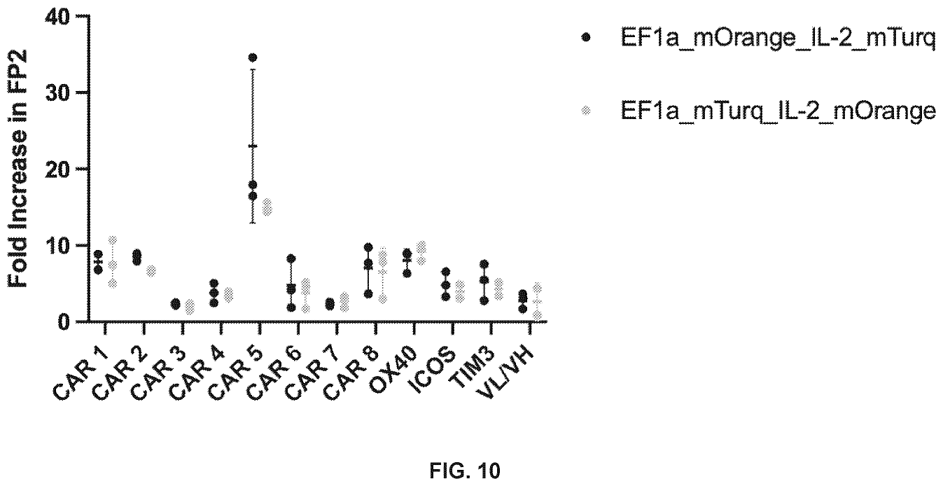

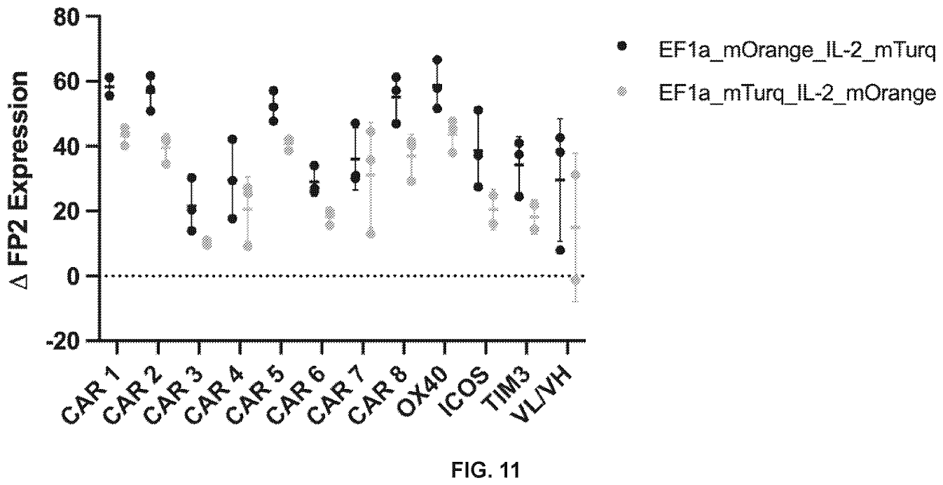

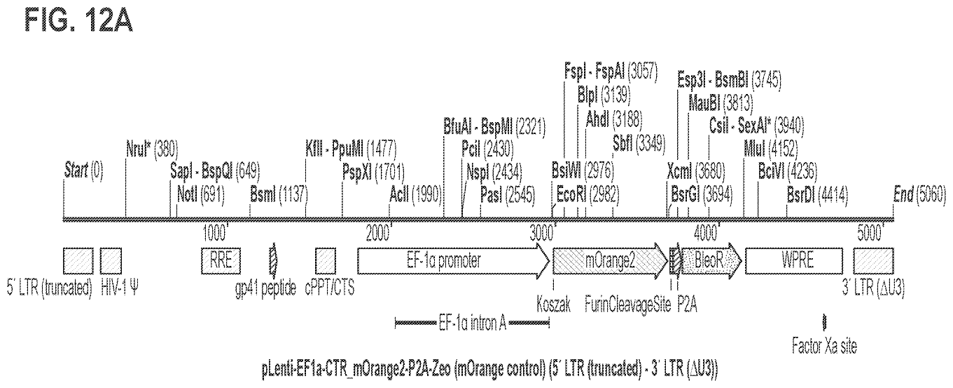

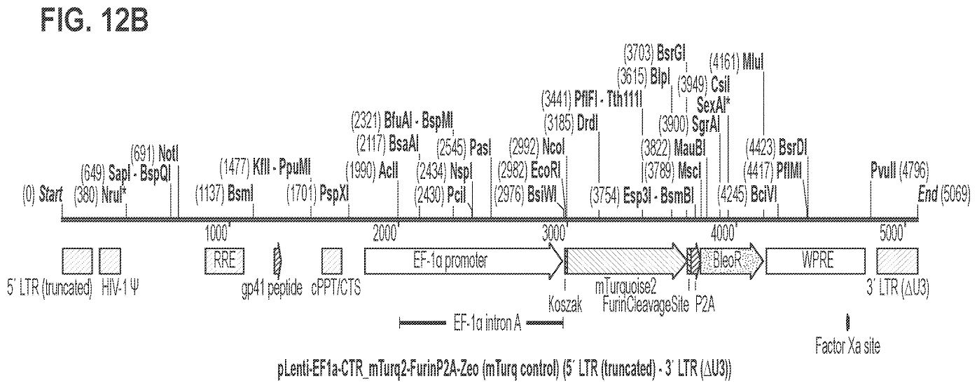

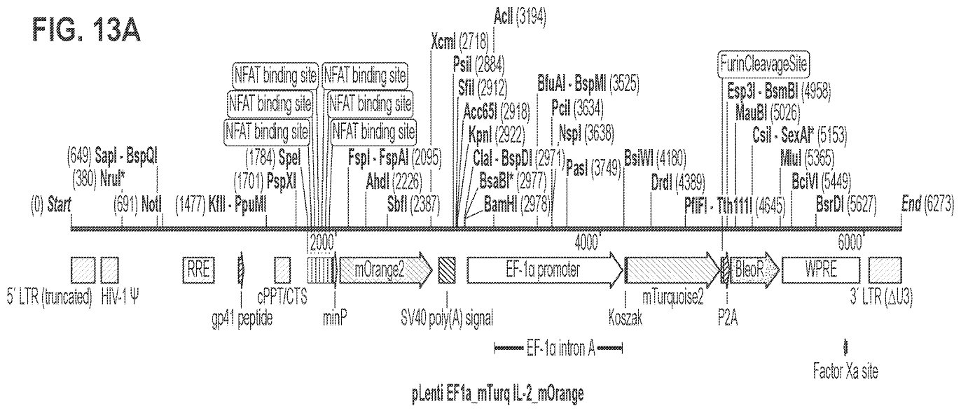

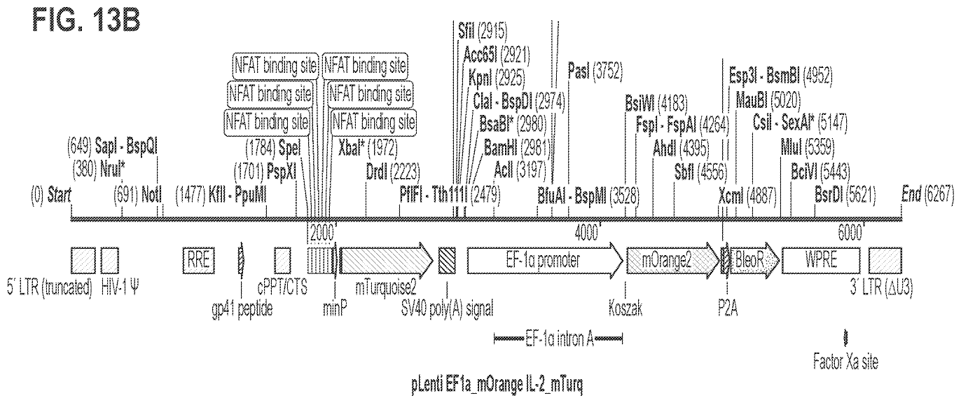

The extent to which the 8 CD33 CARs activate T cells was further evaluated by examining the fold increase in NFAT-inducible fluorescence (FIGS. 10, data in Table 5), the absolute change in NFAT-inducible fluorescence (ΔFP2) (FIG. 11), and the degree to which the transduced Jurkat cells expressed the CD33 CAR (Table 4). As controls, lentiviral vectors encoding known costimulatory or co-inhibitory agents (0X40, ICOS, TIM3, or a VH/VL against CD28) were transduced into Jurkat cells previously transduced with the EF1a_mOrange_IL-2_mTurq or EF1a_mTurq_IL-2_mOrange construct.

TABLE 5
Fold and Delta Increases in FP2 in Tested CARs

| | EF1a_mOrange_IL- | EF1a_mTurq_IL- | EF1a_mOrange_IL- | EF1a_mTurq_IL- |
|---|---|---|---|---|
| CAR | 2_mTurq | 2_mOrange | 2_mTurq | 2_mOrange |
| # | (Fold Increase) | (Fold Increase) | (Delta) | (Delta) |
| 1 | 0.182770672 | 0.36735279 | 0.06768948 | 0.06560674 |
| 2 | 0.060681329 | 0.029963018 | 0.09615783 | 0.11017492 |
| 3 | 0.064981794 | 0.224606151 | 0.3825048 | 0.07078721 |
| 4 | 0.337347505 | 0.131762658 | 0.41099215 | 0.48320023 |
| 5 | 0.43677822 | 0.046825787 | 0.09053537 | 0.04881308 |
| 6 | 0.675153525 | 0.482256984 | 0.15073833 | 0.12741785 |
| 7 | 0.097575115 | 0.270767959 | 0.26618319 | 0.52298681 |
| 8 | 0.437212356 | 0.475518004 | 0.13344737 | 0.18178983 |

The results in FIGS. 10 and 11 show that CARs generated using the anti-CD33 antibodies or fragments there of the disclosure show T cell activating activity in the CAR-IRS assay to varying degrees. For example, anti-CD33-CAR5 shows a high FP2 fold increase (FIG. 10), suggesting a higher T cell activating activity than other CARs tested.

### EXEMPLARY SEQUENCES

Anti-CD33 Single-Domain Antibody Sequences

As used herein, the antibody referred to as sdCD33_2 may also be referred to as “348” or “sdAb348.” The antibody referred to as sdCD33_3 may also be referred to as “353” or “sdAb353.” The antibody referred to as sdCD33_4 may also be referred to as “389” or “sdAb389.” The antibody referred to as sdCD33_5 may also be referred to as “413” or “sdAb413.” The antibody referred to as sdCD33_6 may also be referred to as “416” or “sdAb416.” The antibody referred to as sdCD33_7 may also be referred to as “420” or “sdAb420.” The antibody referred to as sdCD33_8 may also be referred to as “424” or “sdAb424.” The antibody referred to as sdCD33_9 may also be referred to as “426” or “sdAb426.” The antibody referred to as sdCD33_10 may also be referred to as “429” or “sdAb429.” The antibody referred to as sdCD33_11 may also be referred to as “430” or “sdAb430.”

Heavy Chain Variable Region Sequences

sdCD33_1

| | |
|---|---|
| V-D-J Region | QVQLVESGGGSVQAGGSLRLSCAASGRTLRRY |
| | RMGWFRQALGKEREFVAGFTWSGGYAYADSVK |
| | GRFAISGDNAKNTGYLQMNSLKPEDTAVYYCA |
| | LSRQVSLGPGPPNFDYWGQGTQVTVSS |
| | (SEQ ID NO: 1) |
| Heavy Chain FR1 | QVQLVESGGGSVQAGGSLRLSCAAS |
| | (SEQ ID NO: 2) |
| Heavy Chain CDR1 | GRTLRRYR |
| | (SEQ ID NO: 3) |
| Heavy Chain FR2 | MGWFRQALGKEREFVAG |
| | (SEQ ID NO: 4) |
| Heavy Chain CDR2 | FTWSGGY |
| | (SEQ ID NO: 5) |
| Heavy Chain FR3 | AYADSVKGRFAISGDNAKNTGYLQMNSLKPED |
| | TAVYYC |
| | (SEQ ID NO: 6) |
| Heavy Chain CDR3 | ALSRQVSLGPGPPNFDY |
| | (SEQ ID NO: 7) |
| Heavy Chain FR4 | WGQGTQVTVSS |
| | (SEQ ID NO: 8) |

sdCD33_2

| | |
|---|---|
| V-D-J Region | QVQLVQPGGSLRLFCVASEEFFSIYAMGWYRQ |
| | APGKQHEMVARFTRDGKITYADSAKGRFTITR |
| | DAKNTLNLQMNGLIPEDTAVYYCNINHYWGQG |
| | TQVTVSS |
| | (SEQ ID NO: 9) |
| Heavy Chain FR1 | QVQLVQPGGSLRLFCVAS |
| | (SEQ ID NO: 10) |
| Heavy Chain CDR1 | EEFFSIYA |
| | (SEQ ID NO: 11) |
| Heavy Chain FR2 | MGWYRQAPGKQHEMVAR |
| | (SEQ ID NO: 12) |
| Heavy Chain CDR2 | FTRDGKI |
| | (SEQ ID NO: 13) |
| Heavy Chain FR3 | TYADSAKGRFTITRDAKNTLNLQMNGLIPEDT |
| | AVYYC |
| | (SEQ ID NO: 14) |
| Heavy Chain CDR3 | NINHY |
| | (SEQ ID NO: 15) |
| Heavy Chain FR4 | WGQGTQVTVSS |
| | (SEQ ID NO: 16) |

sdCD33_3

| | |
|---|---|
| V-D-J Region | DVQLVESGGGLVQPGGSLRLSCSVSGNIDRFY |
| | AMGWYRQAPGKQRELVAQLTNNEITTYGDSVE |
| | GQFSISGDFDANTVYLQMDSLKPEDTAVYYCH |
| | AHVTTTRWSQDYYWGQGTRVTVSS |
| | (SEQ ID NO: 17) |
| Heavy Chain FR1 | DVQLVESGGGLVQPGGSLRLSCSVS |
| | (SEQ ID NO: 18) |
| Heavy Chain CDR1 | GNIDRFYA |
| | (SEQ ID NO: 19) |
| Heavy Chain FR2 | MGWYRQAPGKQRELVAQ |
| | (SEQ ID NO: 20) |
| Heavy Chain CDR2 | LTNNEIT |
| | (SEQ ID NO: 21) |
| Heavy Chain FR3 | TYGDSVEGQFSISGDFDANTVYLQMDSLKPED |
| | TAVYYC |
| | (SEQ ID NO: 22) |
| Heavy Chain CDR3 | HAHVTTTRWSQDYY |
| | (SEQ ID NO: 23) |
| Heavy Chain FR4 | WGQGTRVTVSS |
| | (SEQ ID NO: 24) |

sdCD33_4

| | |
|---|---|
| V-D-J Region | QVKLEESGGGSVQAGESLRLSCTASGITFRDY |
| | DIDWYRQAPGKEREWLATITPSGTTHYPDSVK |
| | GRATISRDSAKNTVYLQMNSLKPEDTARYECN |
| | TLAYWGSGTQVTVSS |
| | (SEQ ID NO: 25) |
| Heavy Chain FR1 | QVKLEESGGGSVQAGESLRLSCTAS |
| | (SEQ ID NO: 26) |
| Heavy Chain CDR1 | GITFRDYD |
| | (SEQ ID NO: 27) |
| Heavy Chain FR2 | IDWYRQAPGKEREWLAT |
| | (SEQ ID NO: 28) |
| Heavy Chain CDR2 | ITPSGTT |
| | (SEQ ID NO: 29) |
| Heavy Chain FR3 | HYPDSVKGRATISRDSAKNTVYLQMNSLKPED |
| | TARYEC |
| | (SEQ ID NO: 30) |
| Heavy Chain CDR3 | NTLAY |
| | (SEQ ID NO: 31) |
| Heavy Chain FR4 | WGSGTQVTVSS |
| | (SEQ ID NO: 32) |

sdCD33_5

| | |
|---|---|
| V-D-J Region | QVQLQESGGGLVQPGGSLRLSCAASGSVFSIY |
| | AMAWYRQAPGKQRELVAVITSGGATNYADSVK |
| | GRFTISRDIAKKTLYLQMNTLKPEDTAVYYCY |
| | AHLLIQPFDRFYDYWGQGTQVTVSS |
| | (SEQ ID NO: 33) |
| Heavy Chain FR1 | QVQLQESGGGLVQPGGSLRLSCAAS |
| | (SEQ ID NO: 34) |
| Heavy Chain CDR1 | GSVFSIYA |
| | (SEQ ID NO: 35) |
| Heavy Chain FR2 | MAWYRQAPGKQRELVAV |
| | (SEQ ID NO: 36) |
| Heavy Chain CDR2 | ITSGGAT |
| | (SEQ ID NO: 37) |
| Heavy Chain FR3 | NYADSVKGRFTISRDIAKKTLYLQMNTLKPED |
| | TAVYYC |
| | (SEQ ID NO: 38) |
| Heavy Chain CDR3 | YAHLLIQPFDRFYDY |
| | (SEQ ID NO: 39) |
| Heavy Chain FR4 | WGQGTQVTVSS |
| | (SEQ ID NO: 40) |

sdCD33_6

| | |
|---|---|
| V-D-J Region | EVQLVESGGGLVQPRGSLRLSCVVSGSMSSIY |
| | SMSWYRQPPGKQRELVAHITTTGTTNYIDSVK |
| | GRFTISIDNDINVIYLQMNTLKPEDTAVYYCN |
| | AGLKAGPGPRLDYWGLGTQVTVSS |
| | (SEQ ID NO: 41) |
| Heavy Chain FR1 | EVQLVESGGGLVQPRGSLRLSCVVS |
| | (SEQ ID NO: 42) |
| Heavy Chain CDR1 | GSMSSIYS |
| | (SEQ ID NO: 43) |
| Heavy Chain FR2 | MSWYRQPPGKQRELVAH |
| | (SEQ ID NO: 44) |
| Heavy Chain CDR2 | ITTTGTT |
| | (SEQ ID NO: 45) |
| Heavy Chain FR3 | NYIDSVKGRFTISIDNDINVIYLQMNTLKPED |
| | TAVYYC |
| | (SEQ ID NO: 46) |
| Heavy Chain CDR3 | NAGLKAGPGPRLDY |
| | (SEQ ID NO: 47) |
| Heavy Chain FR4 | WGLGTQVTVSS |
| | (SEQ ID NO: 48) |

sdCD33_7

| | |
|---|---|
| V-D-J Region | EVQLVESGGGLVQPGGSLRLSCVASGFTFKDY |
| | GMTWVRQAPGKALEWVSDINSAGDGIYYSDSV |
| | KGRFTISRDDSKGTLYLQMNSLTPEDTAIYYC |
| | AAERQRAGDVKRSLAPITAHIWGQGTQVTVSS |
| | (SEQ ID NO: 49) |
| Heavy Chain FR1 | EVQLVESGGGLVQPGGSLRLSCVAS |
| | (SEQ ID NO: 50) |
| Heavy Chain CDR1 | GFTFKDYG |
| | (SEQ ID NO: 51) |
| Heavy Chain FR2 | MTWVRQAPGKALEWVSD |
| | (SEQ ID NO: 52) |
| Heavy Chain CDR2 | INSAGDGI |
| | (SEQ ID NO: 53) |
| Heavy Chain FR3 | YYSDSVKGRFTISRDDSKGTLYLQMNSLTPED |
| | TAIYYC |
| | (SEQ ID NO: 54) |
| Heavy Chain CDR3 | AAERQRAGDVKRSLAPITAHI |
| | (SEQ ID NO: 55) |
| Heavy Chain FR4 | WGQGTQVTVSS |
| | (SEQ ID NO: 56) |

sdCD33_8

| | |
|---|---|
| V-D-J Region | QVQLVESGGGLVQAGGSLRLSCAASGGAFSTY |
| | TMGWFRQAPGKEREFVAAIAWTGTHTYYSDSV |
| | KGRFTISRDNDKNTVFLQMNSLKPEDTAVYYC |
| | AQASSRYRAVTDSLSENHWGPGTQVTVST |
| | (SEQ ID NO: 57) |
| Heavy Chain FR1 | QVQLVESGGGLVQAGGSLRLSCAAS |
| | (SEQ ID NO: 58) |
| Heavy Chain CDR1 | GGAFSTYT |
| | (SEQ ID NO: 59) |
| Heavy Chain FR2 | MGWFRQAPGKEREFVAA |
| | (SEQ ID NO: 60) |
| Heavy Chain CDR2 | IAWTGTHT |
| | (SEQ ID NO: 61) |
| Heavy Chain FR3 | YYSDSVKGRFTISRDNDKNTVFLQMNSLKPED |
| | TAVYYC |
| | (SEQ ID NO: 62) |
| Heavy Chain CDR3 | AQASSRYRAVTDSLSENH |
| | (SEQ ID NO: 63) |
| Heavy Chain FR4 | WGPGTQVTVST |
| | (SEQ ID NO: 64) |

sdCD33_9

| | |
|---|---|
| V-D-J Region | QVKLEESGGGLVRAGGSLRLSCAASGGTFSTY |
| | AMAWFRQAPGKEREFVAAITWGGGSTYYEDSV |
| | KGRFTISRDNAKNTGVLQMNNLDVDDTAVYYC |
| | YAFGRSRAGDANGDYWGQGTQVTVSS |
| | (SEQ ID NO: 65) |
| Heavy Chain FR1 | QVKLEESGGGLVRAGGSLRLSCAAS |
| | (SEQ ID NO: 66) |
| Heavy Chain CDR1 | GGTFSTYA |
| | (SEQ ID NO: 67) |
| Heavy Chain FR2 | MAWFRQAPGKEREFVAA |
| | (SEQ ID NO: 68) |
| Heavy Chain CDR2 | ITWGGGST |
| | (SEQ ID NO: 69) |
| Heavy Chain FR3 | YYEDSVKGRFTISRDNAKNTGVLQMNNLDVDD |
| | TAVYYC |
| | (SEQ ID NO: 70) |
| Heavy Chain CDR3 | YAFGRSRAGDANGDY |
| | (SEQ ID NO: 71) |
| Heavy Chain FR4 | WGQGTQVTVSS |
| | (SEQ ID NO: 72) |

sdCD33_10

| | |
|---|---|
| V-D-J Region | QVQLVETGGGLVRAGGSLRLSCAASGRTADIY |
| | NIGWFRQAPGKEREFVAAITWIGRTPYYADAV |
| | KGRFAFSTDSAKNTVSLQMDNLKPEDTGVYYC |
| | NAAHYLEGNTDYYWGQGTQVTVSS |
| | (SEQ ID NO: 73) |
| Heavy Chain FR1 | QVQLVETGGGLVRAGGSLRLSCAAS |
| | (SEQ ID NO: 74) |
| Heavy Chain CDR1 | GRTADIYN |
| | (SEQ ID NO: 75) |
| Heavy Chain FR2 | IGWFRQAPGKEREFVAA |
| | (SEQ ID NO: 76) |
| Heavy Chain CDR2 | ITWIGRTP |
| | (SEQ ID NO: 77) |
| Heavy Chain FR3 | YYADAVKGRFAFSTDSAKNTVSLQMDNLKPED |
| | TGVYYC |
| | (SEQ ID NO: 78) |
| Heavy Chain CDR3 | NAAHYLEGNTDYY |
| | (SEQ ID NO: 79) |
| Heavy Chain FR4 | WGQGTQVTVSS |
| | (SEQ ID NO: 80) |

sdCD33_11

| | |
|---|---|
| V-D-J Region | AVQLVESGGGLVQAGGSLRLSCAASGDTFANY |
| | AMGWFRQAPGKEREFVAAISWSDSSTHYADSV |
| | KGRFTIPRDNAKNAVYLQMDQLKPEDMAVYYC |
| | YAYIRGVGEVRYVDYWGQGTQVTVSS |
| | (SEQ ID NO: 81) |
| Heavy Chain FR1 | AVQLVESGGGLVQAGGSLRLSCAAS |
| | (SEQ ID NO: 82) |
| Heavy Chain CDR1 | GDTFANYA |
| | (SEQ ID NO: 83) |
| Heavy Chain FR2 | MGWFRQAPGKEREFVAA |
| | (SEQ ID NO: 84) |
| Heavy Chain CDR2 | ISWSDSST |
| | (SEQ ID NO: 85) |
| Heavy Chain FR3 | HYADSVKGRFTIPRDNAKNAVYLQMDQLKPED |
| | MAVYYC |
| | (SEQ ID NO: 86) |
| Heavy Chain CDR3 | YAYIRGVGEVRYVDY |
| | (SEQ ID NO: 87) |
| Heavy Chain FR4 | WGQGTQVTVSS |
| | (SEQ ID NO: 88) |

TABLE 4
sdAbs Tested in Example 3 and Exemplary
Sequences Contained Therein

| Exemplary | |
|---|---|
| Sequence Name | sdAb of Example 3 |
| sdCD33_1 | Hu195 |
| sdCD33_2 | 348 |
| sdCD33_3 | 353 |
| sdCD33_4 | 389 |
| sdCD33_5 | 413 |
| sdCD33_6 | 416 |
| sdCD33_7 | 420 |
| sdCD33_8 | 424 |
| sdCD33_9 | 426 |
| sdCD33_10 | 429 |
| sdCD33_11 | 430 |

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An anti-CD33 antibody, or antigen-binding fragment thereof, comprising an amino acid sequence selected from a group consisting of SEQ ID NO: 1-88.

2. An anti-CD33 antibody, or antigen-binding fragment thereof, comprising a CDR sequence encompassed within any one of SEQ ID NO: 1-88.

3. An anti-CD33 antibody, or antigen-binding fragment thereof, comprising CDR1, CDR2, and CDR3 encompassed within any one of SEQ ID NO: 1, 9, 17, 25, 33, 41, 49, 57, 65, 73, or 81.

4. An anti-CD33 antibody, or antigen-binding fragment thereof, comprising at least one CDR (e.g., CDR1, CDR2, and/or CDR3) depicted in any one of SEQ ID NO: 1-88.

5. An anti-CD33 antibody, or antigen-binding fragment thereof, comprising at least one CDR that is at least 85%, 90%, 91%, 92%, 93%, 94%, 95%, 96%, 97%, 98%, 99%, or 100% identical to a CDR (e.g., CDR1, CDR2, and/or CDR3) depicted in any one of SEQ ID NO: 1-88.

6. An anti-CD33 antibody, or antigen-binding fragment thereof, comprising a VHH comprising an amino acid sequence selected from a group consisting of SEQ ID NO: 1-88.

7. An anti-CD33 antibody, or antigen-binding fragment thereof, comprising a VHH comprising a CDR sequence encompassed within any one of SEQ ID NO: 1-88.

8. An anti-CD33 antibody, or antigen-binding fragment thereof, comprising a VHH comprising CDR1, CDR2, and CDR3 encompassed within any one of SEQ ID NO: 1, 9, 17, 25, 33, 41, 49, 57, 65, 73, or 81.

9. An anti-CD33 antibody, or antigen-binding fragment thereof, comprising a VHH comprising at least one CDR (e.g., CDR1, CDR2, and/or CDR3) depicted in any one of SEQ ID NO: 1-88.

10. An anti-CD33 antibody, or antigen-binding fragment thereof, comprising a VHH comprising at least one CDR that is at least 85%, 90%, 91%, 92%, 93%, 94%, 95%, 96%, 97%, 98%, 99%, or 100% identical to a CDR (e.g., CDR1, CDR2, and/or CDR3) depicted in any one of SEQ ID NO: 1-88.

11. The anti-CD33 antibody, or antigen-binding fragment thereof, of any one of claims 1-10, wherein the antibody, or antigen-binding fragment thereof, is a monoclonal antibody, or antigen-binding fragment thereof.

12. The anti-CD33 antibody, or antigen-binding fragment thereof, of any one of claims 1-11, wherein the antibody, or antigen-binding fragment thereof, is a humanized antibody, or antigen-binding fragment thereof.

13. An anti-CD33 antibody, or antigen-binding fragment thereof, that competes with the antibody, or antigen-binding fragment thereof, of any one of claims 1-12.

14. The anti-CD33 antibody, or antigen-binding fragment thereof, of any one of claims 1-13, wherein the antibody, or antigen-binding fragment thereof, comprises a CH2 constant domain and a CH3 constant domain.

15. The anti-CD33 antibody, or antigen-binding fragment thereof, of claim 14, wherein the antibody, or antigen-binding fragment thereof, comprises an amino acid sequence of SEQ ID NO: 89.

16. The anti-CD33 antibody, or antigen-binding fragment thereof, of any one of claims 1-15, wherein the antibody, or antigen-binding fragment thereof, is a heavy chain antibody.

17. The anti-CD33 antibody, or antigen-binding fragment thereof, of any one of claims 1-16, wherein the antibody, or antigen-binding fragment thereof, is a camelid antibody.

18. A chimeric antigen receptor comprising any of the antibody, or antigen-binding fragment thereof, of any one of claims 1-17.

19. A cell expressing the chimeric antigen receptor of claim 18.

20. The cell of claim 19, wherein the cell is an immune effector cell.

21. The cell of claim 19 or 20, wherein the cell is a lymphocyte.

22. The cell of any one of claims 19-22, wherein the cell is a T-cell.

23. The cell of claim 19 or 20, wherein the cell is an NK cell.

24. A nucleic acid, comprising a nucleic acid sequence encoding an antibody, or antigen-binding fragment thereof, of any one of claims 1-17, or the chimeric antigen receptor of claim 18.

25. A vector comprising the nucleic acid of claim 24.

26. A cell comprising the nucleic acid of claim 24 or the vector of claim 25.

27. The cell of claim 26, wherein the cell is an immune cell.

28. The cell of claim 27, wherein the immune cell is selected from the group consisting of a T cell, a Natural Killer (NK) cell, a cytotoxic T lymphocyte (CTL), and a regulatory T cell.

29. A method of producing an antibody, or antigen-binding fragment thereof, comprising culturing the cell of any one of claim 19-23 or 26-28 under conditions suitable for expression of the antibody or antigen-binding fragment thereof.

30. A method of treating a CD33-associated disease or disorder, the method comprising administering to a subject in need thereof an effective amount of the antibody, or antigen-binding fragment thereof, of any one of claims 1-13, or the cell of any one of claim 19-23 or 26-28.

31. A method of treating a subject having or at risk of a neoplastic disease or malignancy of the blood that is associated with CD33 expression, the method comprising administering to the subject a therapeutically effective amount of the antibody, or antigen-binding fragment thereof, of any one of claims 1-13, or the cell of any one of claim 19-23 or 26-28.

32. The method of claim 31, wherein the neoplastic disease or malignancy of the blood that is associated with CD33 expression is myelodysplastic syndrome (MDS), acute myeloid leukemia (AML), multiple myeloma (MM), or a combination thereof.

33. The method of claim 31 or 32, further comprising administering to the subject an effective amount of a chemotherapeutic agent or an oncolytic therapeutic agent.


SEQUENCE LISTING (111 sequences)

SEQ ID NO: 1 - protein, 123 aa, Artificial Sequence
  Synthetic
QVQLVESGGGSVQAGGSLRLSCAASGRTLRRYRMGWFRQALGKEREFVAGFTWSGGYAYADSVKGRFAISGDNAKNTGYLQMNSLKPEDTAVYYCALSRQVSLGPGPPNFDYWGQGTQVTVSS

SEQ ID NO: 2 - protein, 25 aa, Artificial Sequence
  Synthetic
QVQLVESGGGSVQAGGSLRLSCAAS

SEQ ID NO: 3 - protein, 8 aa, Artificial Sequence
  Synthetic
GRTLRRYR

SEQ ID NO: 4 - protein, 17 aa, Artificial Sequence
  Synthetic
MGWFRQALGKEREFVAG

SEQ ID NO: 5 - protein, 7 aa, Artificial Sequence
  Synthetic
FTWSGGY

SEQ ID NO: 6 - protein, 38 aa, Artificial Sequence
  Synthetic
AYADSVKGRFAISGDNAKNTGYLQMNSLKPEDTAVYYC

SEQ ID NO: 7 - protein, 17 aa, Artificial Sequence
  Synthetic
ALSRQVSLGPGPPNFDY

SEQ ID NO: 8 - protein, 11 aa, Artificial Sequence
  Synthetic
WGQGTQVTVSS

SEQ ID NO: 9 - protein, 103 aa, Artificial Sequence
  Synthetic
QVQLVQPGGSLRLFCVASEEFFSIYAMGWYRQAPGKQHEMVARFTRDGKITYADSAKGRFTITRDAKNTLNLQMNGLIPEDTAVYYCNINHYWGQGTQVTVSS

SEQ ID NO: 10 - protein, 18 aa, Artificial Sequence
  Synthetic
QVQLVQPGGSLRLFCVAS

SEQ ID NO: 11 - protein, 8 aa, Artificial Sequence
  Synthetic
EEFFSIYA

SEQ ID NO: 12 - protein, 17 aa, Artificial Sequence
  Synthetic
MGWYRQAPGKQHEMVAR

SEQ ID NO: 13 - protein, 7 aa, Artificial Sequence
  Synthetic
FTRDGKI

SEQ ID NO: 14 - protein, 37 aa, Artificial Sequence
  Synthetic
TYADSAKGRFTITRDAKNTLNLQMNGLIPEDTAVYYC

SEQ ID NO: 15 - protein, 5 aa, Artificial Sequence
  Synthetic
NINHY

SEQ ID NO: 16 - protein, 11 aa, Artificial Sequence
  Synthetic
WGQGTQVTVSS

SEQ ID NO: 17 - protein, 120 aa, Artificial Sequence
  Synthetic
DVQLVESGGGLVQPGGSLRLSCSVSGNIDRFYAMGWYRQAPGKQRELVAQLTNNEITTYGDSVEGQFSISGDFDANTVYLQMDSLKPEDTAVYYCHAHVTTTRWSQDYYWGQGTRVTVSS

SEQ ID NO: 18 - protein, 25 aa, Artificial Sequence
  Synthetic
DVQLVESGGGLVQPGGSLRLSCSVS

SEQ ID NO: 19 - protein, 8 aa, Artificial Sequence
  Synthetic
GNIDRFYA

SEQ ID NO: 20 - protein, 17 aa, Artificial Sequence
  Synthetic
MGWYRQAPGKQRELVAQ

SEQ ID NO: 21 - protein, 7 aa, Artificial Sequence
  Synthetic
LTNNEIT

SEQ ID NO: 22 - protein, 38 aa, Artificial Sequence
  Synthetic
TYGDSVEGQFSISGDFDANTVYLQMDSLKPEDTAVYYC

SEQ ID NO: 23 - protein, 14 aa, Artificial Sequence
  Synthetic
HAHVTTTRWSQDYY

SEQ ID NO: 24 - protein, 11 aa, Artificial Sequence
  Synthetic
WGQGTRVTVSS

SEQ ID NO: 25 - protein, 111 aa, Artificial Sequence
  Synthetic
QVKLEESGGGSVQAGESLRLSCTASGITFRDYDIDWYRQAPGKEREWLATITPSGTTHYPDSVKGRATISRDSAKNTVYLQMNSLKPEDTARYECNTLAYWGSGTQVTVSS

SEQ ID NO: 26 - protein, 25 aa, Artificial Sequence
  Synthetic
QVKLEESGGGSVQAGESLRLSCTAS

SEQ ID NO: 27 - protein, 8 aa, Artificial Sequence
  Synthetic
GITFRDYD

SEQ ID NO: 28 - protein, 17 aa, Artificial Sequence
  Synthetic
IDWYRQAPGKEREWLAT

SEQ ID NO: 29 - protein, 7 aa, Artificial Sequence
  Synthetic
ITPSGTT

SEQ ID NO: 30 - protein, 38 aa, Artificial Sequence
  Synthetic
HYPDSVKGRATISRDSAKNTVYLQMNSLKPEDTARYEC

SEQ ID NO: 31 - protein, 5 aa, Artificial Sequence
  Synthetic
NTLAY

SEQ ID NO: 32 - protein, 11 aa, Artificial Sequence
  Synthetic
WGSGTQVTVSS

SEQ ID NO: 33 - protein, 121 aa, Artificial Sequence
  Synthetic
QVQLQESGGGLVQPGGSLRLSCAASGSVFSIYAMAWYRQAPGKQRELVAVITSGGATNYADSVKGRFTISRDIAKKTLYLQMNTLKPEDTAVYYCYAHLLIQPFDRFYDYWGQGTQVTVSS

SEQ ID NO: 34 - protein, 25 aa, Artificial Sequence
  Synthetic
QVQLQESGGGLVQPGGSLRLSCAAS

SEQ ID NO: 35 - protein, 8 aa, Artificial Sequence
  Synthetic
GSVFSIYA

SEQ ID NO: 36 - protein, 17 aa, Artificial Sequence
  Synthetic
MAWYRQAPGKQRELVAV

SEQ ID NO: 37 - protein, 7 aa, Artificial Sequence
  Synthetic
ITSGGAT

SEQ ID NO: 38 - protein, 38 aa, Artificial Sequence
  Synthetic
NYADSVKGRFTISRDIAKKTLYLQMNTLKPEDTAVYYC

SEQ ID NO: 39 - protein, 15 aa, Artificial Sequence
  Synthetic
YAHLLIQPFDRFYDY

SEQ ID NO: 40 - protein, 11 aa, Artificial Sequence
  Synthetic
WGQGTQVTVSS

SEQ ID NO: 41 - protein, 120 aa, Artificial Sequence
  Synthetic
EVQLVESGGGLVQPRGSLRLSCVVSGSMSSIYSMSWYRQPPGKQRELVAHITTTGTTNYIDSVKGRFTISIDNDINVIYLQMNTLKPEDTAVYYCNAGLKAGPGPRLDYWGLGTQVTVSS

SEQ ID NO: 42 - protein, 25 aa, Artificial Sequence
  Synthetic
EVQLVESGGGLVQPRGSLRLSCVVS

SEQ ID NO: 43 - protein, 8 aa, Artificial Sequence
  Synthetic
GSMSSIYS

SEQ ID NO: 44 - protein, 17 aa, Artificial Sequence
  Synthetic
MSWYRQPPGKQRELVAH

SEQ ID NO: 45 - protein, 7 aa, Artificial Sequence
  Synthetic
ITTTGTT

SEQ ID NO: 46 - protein, 38 aa, Artificial Sequence
  Synthetic
NYIDSVKGRFTISIDNDINVIYLQMNTLKPEDTAVYYC

SEQ ID NO: 47 - protein, 14 aa, Artificial Sequence
  Synthetic
NAGLKAGPGPRLDY

SEQ ID NO: 48 - protein, 11 aa, Artificial Sequence
  Synthetic
WGLGTQVTVSS

SEQ ID NO: 49 - protein, 128 aa, Artificial Sequence
  Synthetic
EVQLVESGGGLVQPGGSLRLSCVASGFTFKDYGMTWVRQAPGKALEWVSDINSAGDGIYYSDSVKGRFTISRDDSKGTLYLQMNSLTPEDTAIYYCAAERQRAGDVKRSLAPITAHIWGQGTQVTVSS

SEQ ID NO: 50 - protein, 25 aa, Artificial Sequence
  Synthetic
EVQLVESGGGLVQPGGSLRLSCVAS

SEQ ID NO: 51 - protein, 8 aa, Artificial Sequence
  Synthetic
GFTFKDYG

SEQ ID NO: 52 - protein, 17 aa, Artificial Sequence
  Synthetic
MTWVRQAPGKALEWVSD

SEQ ID NO: 53 - protein, 8 aa, Artificial Sequence
  Synthetic
INSAGDGI

SEQ ID NO: 54 - protein, 38 aa, Artificial Sequence
  Synthetic
YYSDSVKGRFTISRDDSKGTLYLQMNSLTPEDTAIYYC

SEQ ID NO: 55 - protein, 21 aa, Artificial Sequence
  Synthetic
AAERQRAGDVKRSLAPITAHI

SEQ ID NO: 56 - protein, 11 aa, Artificial Sequence
  Synthetic
WGQGTQVTVSS

SEQ ID NO: 57 - protein, 125 aa, Artificial Sequence
  Synthetic
QVQLVESGGGLVQAGGSLRLSCAASGGAFSTYTMGWFRQAPGKEREFVAAIAWTGTHTYYSDSVKGRFTISRDNDKNTVFLQMNSLKPEDTAVYYCAQASSRYRAVTDSLSENHWGPGTQVTVST

SEQ ID NO: 58 - protein, 25 aa, Artificial Sequence
  Synthetic
QVQLVESGGGLVQAGGSLRLSCAAS

SEQ ID NO: 59 - protein, 8 aa, Artificial Sequence
  Synthetic
GGAFSTYT

SEQ ID NO: 60 - protein, 17 aa, Artificial Sequence
  Synthetic
MGWFRQAPGKEREFVAA

SEQ ID NO: 61 - protein, 8 aa, Artificial Sequence
  Synthetic
IAWTGTHT

SEQ ID NO: 62 - protein, 38 aa, Artificial Sequence
  Synthetic
YYSDSVKGRFTISRDNDKNTVFLQMNSLKPEDTAVYYC

SEQ ID NO: 63 - protein, 18 aa, Artificial Sequence
  Synthetic
AQASSRYRAVTDSLSENH

SEQ ID NO: 64 - protein, 11 aa, Artificial Sequence
  Synthetic
WGPGTQVTVST

SEQ ID NO: 65 - protein, 122 aa, Artificial Sequence
  Synthetic
QVKLEESGGGLVRAGGSLRLSCAASGGTFSTYAMAWFRQAPGKEREFVAAITWGGGSTYYEDSVKGRFTISRDNAKNTGVLQMNNLDVDDTAVYYCYAFGRSRAGDANGDYWGQGTQVTVSS

SEQ ID NO: 66 - protein, 25 aa, Artificial Sequence
  Synthetic
QVKLEESGGGLVRAGGSLRLSCAAS

SEQ ID NO: 67 - protein, 8 aa, Artificial Sequence
  Synthetic
GGTFSTYA

SEQ ID NO: 68 - protein, 17 aa, Artificial Sequence
  Synthetic
MAWFRQAPGKEREFVAA

SEQ ID NO: 69 - protein, 8 aa, Artificial Sequence
  Synthetic
ITWGGGST

SEQ ID NO: 70 - protein, 38 aa, Artificial Sequence
  Synthetic
YYEDSVKGRFTISRDNAKNTGVLQMNNLDVDDTAVYYC

SEQ ID NO: 71 - protein, 15 aa, Artificial Sequence
  Synthetic
YAFGRSRAGDANGDY

SEQ ID NO: 72 - protein, 11 aa, Artificial Sequence
  Synthetic
WGQGTQVTVSS

SEQ ID NO: 73 - protein, 120 aa, Artificial Sequence
  Synthetic
QVQLVETGGGLVRAGGSLRLSCAASGRTADIYNIGWFRQAPGKEREFVAAITWIGRTPYYADAVKGRFAFSTDSAKNTVSLQMDNLKPEDTGVYYCNAAHYLEGNTDYYWGQGTQVTVSS

SEQ ID NO: 74 - protein, 25 aa, Artificial Sequence
  Synthetic
QVQLVETGGGLVRAGGSLRLSCAAS

SEQ ID NO: 75 - protein, 8 aa, Artificial Sequence
  Synthetic
GRTADIYN

SEQ ID NO: 76 - protein, 17 aa, Artificial Sequence
  Synthetic
IGWFRQAPGKEREFVAA

SEQ ID NO: 77 - protein, 8 aa, Artificial Sequence
  Synthetic
ITWIGRTP

SEQ ID NO: 78 - protein, 38 aa, Artificial Sequence
  Synthetic
YYADAVKGRFAFSTDSAKNTVSLQMDNLKPEDTGVYYC

SEQ ID NO: 79 - protein, 13 aa, Artificial Sequence
  Synthetic
NAAHYLEGNTDYY

SEQ ID NO: 80 - protein, 11 aa, Artificial Sequence
  Synthetic
WGQGTQVTVSS

SEQ ID NO: 81 - protein, 122 aa, Artificial Sequence
  Synthetic
AVQLVESGGGLVQAGGSLRLSCAASGDTFANYAMGWFRQAPGKEREFVAAISWSDSSTHYADSVKGRFTIPRDNAKNAVYLQMDQLKPEDMAVYYCYAYIRGVGEVRYVDYWGQGTQVTVSS

SEQ ID NO: 82 - protein, 25 aa, Artificial Sequence
  Synthetic
AVQLVESGGGLVQAGGSLRLSCAAS

SEQ ID NO: 83 - protein, 8 aa, Artificial Sequence
  Synthetic
GDTFANYA

SEQ ID NO: 84 - protein, 17 aa, Artificial Sequence
  Synthetic
MGWFRQAPGKEREFVAA

SEQ ID NO: 85 - protein, 8 aa, Artificial Sequence
  Synthetic
ISWSDSST

SEQ ID NO: 86 - protein, 38 aa, Artificial Sequence
  Synthetic
HYADSVKGRFTIPRDNAKNAVYLQMDQLKPEDMAVYYC

SEQ ID NO: 87 - protein, 15 aa, Artificial Sequence
  Synthetic
YAYIRGVGEVRYVDY

SEQ ID NO: 88 - protein, 11 aa, Artificial Sequence
  Synthetic
WGQGTQVTVSS

SEQ ID NO: 89 - protein, 217 aa, Artificial Sequence
  Synthetic
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 356 aa, Artificial Sequence
  Synthetic
MELGLSWVVLAALLQGVQAQVKLEESGGGSVQAGESLRLSCTASGITFRDYDIDWYRQAPGKEREWLATITPSGTTHYPDSVKGRATISRDSAKNTVYLQMNSLKPEDTARYECNTLAYWGSGTQVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 365 aa, Artificial Sequence
  Synthetic
MELGLSWVVLAALLQGVQAQVQLVETGGGLVRAGGSLRLSCAASGRTADIYNIGWFRQAPGKEREFVAAITWIGRTPYYADAVKGRFAFSTDSAKNTVSLQMDNLKPEDTGVYYCNAAHYLEGNTDYYWGQGTQVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - protein, 348 aa, Artificial Sequence
  Synthetic
MELGLSWVVLAALLQGVQAQVQLVQPGGSLRLFCVASEEFFSIYAMGWYRQAPGKQHEMVARFTRDGKITYADSAKGRFTITRDAKNTLNLQMNGLIPEDTAVYYCNINHYWGQGTQVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 365 aa, Artificial Sequence
  Synthetic
MELGLSWVVLAALLQGVQADVQLVESGGGLVQPGGSLRLSCSVSGNIDRFYAMGWYRQAPGKQRELVAQLTNNEITTYGDSVEGQFSISGDFDANTVYLQMDSLKPEDTAVYYCHAHVTTTRWSQDYYWGQGTRVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - DNA, 30 bp, Artificial Sequence
  Synthetic
ggaggaaaaactgtttcatacagaaggcgt

SEQ ID NO: 95 - DNA, 31 bp, Artificial Sequence
  Synthetic
tagagggtatataatggaagctcgaattcca

SEQ ID NO: 96 - DNA, 221 bp, Artificial Sequence
  Synthetic
ggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtgatctagacttagagggtatataatggaagctcgaattcca

SEQ ID NO: 97 - DNA, 1179 bp, Artificial Sequence
  Synthetic
ggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 98 - DNA, 717 bp, Artificial Sequence
  Synthetic
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgtcctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactactttagcgacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaag

SEQ ID NO: 99 - DNA, 708 bp, Artificial Sequence
  Synthetic
atggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgctaagctgaaggtgaccaagggtggccccctgcccttcgcctgggacatcctgtcccctcatttcacctacggctccaaggcctacgtgaagcaccccgccgacatccccgactacttcaagctgtccttccccgagggcttcaagtgggagcgcgtgatgaactacgaggacggcggcgtggtgaccgtgacccaggactcctccctgcaggacggcgagttcatctacaaggtgaagctgcgcggcaccaacttcccctccgacggccccgtgatgcagaagaagaccatgggctgggaggcctcctccgagcggatgtaccccgaggacggtgccctgaagggcaagatcaagatgaggctgaagctgaaggacggcggccactacacctccgaggtcaagaccacctacaaggccaagaagcccgtgcagctgcccggcgcctacatcgtcgacatcaagttggacatcacctcccacaacgaggactacaccatcgtggaacagtacgaacgcgccgagggccgccactccaccggcggcatggacgagctgtacaag

SEQ ID NO: 100 - protein, 5 aa, Artificial  Sequence
  Synthetic
GGGGS

SEQ ID NO: 101 - protein, 10 aa, Artificial Sequence
  Synthetic
GGGGSGGGGS

SEQ ID NO: 102 - protein, 15 aa, Artificial Sequence
  Synthetic
GGGGSGGGGSGGGGS

SEQ ID NO: 103 - protein, 45 aa, Artificial Sequence
  Synthetic
TTTPAPRPPTPAPTIASQPLSLRPEACRPAAGGAVHTRGLDFACD

SEQ ID NO: 104 - protein, 42 aa, Artificial Sequence
  Synthetic
AAAIEVMYPPPYLDNEKSNGTIIHVKGKHLCPSPLFPGPSKP

SEQ ID NO: 105 - protein, 2 aa, Artificial Sequence
  Synthetic
  REPEAT (1)..(1): May be repeated 3-12 times
  REPEAT (1)..(2): May be repeated 3-12 times
GS

SEQ ID NO: 106 - protein, 24 aa, Artificial Sequence
  Synthetic
IYIWAPLAGTCGVLLLSLVITLYC

SEQ ID NO: 107 - protein, 68 aa, Artificial Sequence
  Synthetic
FWVLVVVGGVLACYSLLVTVAFIIFWVRSKRSRLLHSDYMNMTPRRPGPTRKHYQPYAPPRDFAAYRS

SEQ ID NO: 108 - protein, 112 aa, Artificial Sequence
  Synthetic
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 109 - protein, 112 aa, Artificial Sequence
  Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 110 - protein, 42 aa, Artificial Sequence
  Synthetic
KRGRKKLLYIFKQPFMRPVQTTQEEDGCSCRFPEEEEGGCEL

SEQ ID NO: 111 - protein, 2 aa, Artificial Sequence
  Synthetic
  REPEAT (1)..(1): May be repeated 1-10 times
  REPEAT (1)..(2): May be repeated 1-5 times
  REPEAT (2)..(2): May be repeated 1-3 times
GS